(19) United States
(12) Patent Application Publication
(10) Pub. No.: US 2009/0298799 A1
(43) Pub. Date: Dec. 3, 2009

(54) Methods of Treating Osteoporosis and Secondary Hyperparathyroidism Using 20-Methyl, Gemini Vitamin D3 Compounds

(75) Inventors: Luciano Adorini, Milan (IT); Milan R. Uskokovic, Upper Montclair, NJ (US); Michèle Resche-Rignon, Paris (FR)

Correspondence Address: EDWARDS ANGELL PALMER & DODGE LLP, P.O. BOX 55874, BOSTON, MA, 02205, US

(73) Assignees: BioXell S.p.A., Milan (IT); Galapagos SASU, Romainville (FR)

(21) Appl. No.: 11/886,922

(22) PCT Filed: Mar. 23, 2006
(86) PCT No.: PCT/IB2006/001541

§ 371 Date: Nov. 20, 2008

Related U.S. Application Data
(60) Provisional application No. 60/664,397, filed on Mar. 23, 2005.

(51) Int. Cl. A61K 31/593

(52) U.S. Cl. 514/167

(57) ABSTRACT

The invention provides for methods of using 20-methyl Gemini vitamin D₃ compounds to treat osteoporosis and secondary hyperparathyroidism.

### RELATED APPLICATIONS

This application claims the benefit of U.S. provisional patent application No. 60/664,397, filed Mar. 23, 2005. This application is related to international patent application No. PCT/US2006/______, filed on Mar. 23, 2006 (Attorney Docket No. 49949-63097PCT(A), Express Mail Label No. EV 756031949 US). The disclosures of both applications are incorporated herein in their entireties by this reference.

### BACKGROUND OF THE INVENTION

The importance of vitamin D (cholecalciferol) in the biological systems of higher animals has been recognized since its discovery by Mellanby in 1920 (Mellanby, E. (1921) Spec. Rep. Ser. Med. Res. Council (GB) SRS 61:4). It was in the interval of 1920-1930 that vitamin D officially became classified as a “vitamin” essential for the normal development of the skeleton and maintenance of calcium and phosphorous homeostasis.

Studies involving the metabolism of vitamin D₃ were initiated with the discovery and chemical characterization of the plasma metabolite, 25-hydroxyvitamin D₃ [25(OH)D₃] (Blunt, J. W. et al. (1968) Biochemistry 6:3317-3322) and the hormonally active form, 1α,25(OH)₂D₃ (Myrtle, J. F et al. (1970) J. Biol. Chem. 245:1190-1196; Norman, A. W. et al. (1971) Science 173:51-54; Lawson, D. E. M. et al. (1971) Nature 230:228-230; Holick, M. F. (1971) Proc. Natl. Acad J. Sci. USA 68:803-804). The formulation of the concept of a vitamin D endocrine system was dependent upon the appreciation of the key role of the kidney in producing lox, 25(OH)₂D₃ in a carefully regulated fashion (Fraser, D. R. and Kodicek, E (1970) Nature 288:764-766; Wong, R. G. et al. (1972) J. Clin. Invest. 51: 1287-1291), and the discovery of a nuclear receptor for 1 α,25(OH)₂D₃ (VD₃R) in the intestine (Haussler, M. R. et al. (1969) Exp. Cell Res. 58-234-242; Tsai, H. C. and Norman, A. W. (1972) J. Biol. Chem. 248:5967-5975).

The operation of the vitamin D endocrine system depends on the following: first, on the presence of cytochrome P450 enzymes in the liver (Bergman, T. and Postlind, H. (1991) Biochem. J. 276:427-432; Ohyama, Y. and Okuda, K. (1991) J. Biol. Chem. 266:8690-8695) and kidney (Henry, H. L. and Norman, A. W. (1974) J. Biol. Chem, 249:7529-7535; Gray, R. W. and Ghazarian, J. G. (1989) Biochem. J. 259:561-568), and in a variety of other tissues to effect the conversion of vitamin D₃ into biologically active metabolites such as 1α,25(OH)₂D₃ and 24R,25(OH)₂D₃; second, on the existence of the plasma vitamin D binding protein (DBP) to effect the selective transport and delivery of these hydrophobic molecules to the various tissue components of the vitamin D endocrine system (Van Baelen, H. et al. (1988) Ann NY Acad. Sci. 538:60-68; Cooke, N. E. and Haddad, J. G. (1989) Endocr. Rev. 10:294-307; Bikle, D. D. et al. (1986) J. Clin. Endocrinol. Metab. 63:954-959); and third, upon the existence of stereoselective receptors in a wide variety of target tissues that interact with the agonist 1α,25(OH)₂D₃ to generate the requisite specific biological responses for this secosteroid hormone (Pike, J. W. (1991) Ann. Rev. Nutr. 11: 189-216). To date, there is evidence that nuclear receptors for 1α,25(OH)₂D₃ (VD₃R) exist in more than 30 tissues and cancer cell lines (Reichel, H. and Norman, A. W. (1989) Annu. Rev. Med. 40:71-78).

Vitamin D₃ and its hormonally active forms are well-known regulators of calcium and phosphorous homeostasis. These compounds are known to stimulate, at least one of, intestinal absorption of calcium and phosphate, mobilization of bone mineral, and retention of calcium in the kidneys. Furthermore, the discovery of the presence of specific vitamin D receptors in more than 30 tissues has led to the identification of vitamin D₃ as a pluripotent regulator outside its classical role in calcium/bone homeostasis. A paracrine role for 1α,25(OH)₂ D₃ has been suggested by the combined presence of enzymes capable of oxidizing vitamin D₃ into its active forms, e.g., 25-OHD-1α-hydroxylase, and specific receptors in several tissues such as bone, keratinocytes, placenta, and immune cells. Moreover, vitamin D₃ hormone and active metabolites have been found to be capable of regulating cell proliferation and differentiation of both normal and malignant cells (Reichel, H. et al. (1989) Ann. Rev. Med. 40: 71-78).

Given the activities of vitamin D₃ and its metabolites, much attention has focused on the development of synthetic analogs of these compounds. A large number of these analogs involve structural modifications in the A ring, B ring, C/D rings, and, primarily, the side chain (Bouillon, R. et al., Endocrine Reviews 16(2):201-204). Although a vast majority of the vitamin D₃ analogs developed to date involve structural modifications in the side chain, a few studies have reported the biological profile of A-ring diastereomers (Norman, A. W. et al. J. Biol. Chem. 268 (27): 20022-20030). Furthermore, biological esterification of steroids has been studied (Hochberg, R. B., (1998) Endocr Rev. 19(3): 331-348), and esters of vitamin D₃ are known (WO 97/11053).

Moreover, despite much effort in developing synthetic analogs, clinical applications of vitamin D and its structural analogs have been limited by the undesired side effects elicited by these compounds after administration to a subject for known indications/applications of vitamin D compounds. Therefore, structural analogs of vitamin D having improved therapeutic activity, particularly for the treatment of osteoporosis and secondary hyperparathyroidism and/or reduced undesirable side effects are needed.

### SUMMARY OF THE INVENTION

The invention provides novel vitamin D₃ compounds having improved therapeutic activity for the treatment of osteoporosis and secondary hyperparathyroidism and/or reduced undesirable side effects useful for the treatment of osteoporosis and secondary.

Thus, in one aspect, the invention provides a method for treating osteoporosis in a subject comprising administering to a subject in need thereof a therapeurically effective amount of a vitamin D₃ compound selected from the group consisting of (20S)-1,25-Dihydroxy-20-(5,5,5-trifluoro-4-hydroxy-4-trifluoromethyl-pent-2-ynyl)-cholecalciferol (1); (20S)-1,25-Dihydroxy-20-[(2E)-5,5,5-trifluoro-4-hydroxy-4-trifluoromethyl-pent-2-enyl]-cholecalciferol (3); and (20S)-1α-Fluoro-25-hydroxy-20-(5,5,5-trifluoro-4-hydroxy-4-trifluoromethyl-pent-2-ynyl)-cholecalciferol (6), thereby treating the subject for osteoporosis.

In another aspect, the invention provides a method for treating a subject for secondary hyperparathyroidism comprising administering to a subject in need thereof a therapeutically effective amount of a vitamin D₃ compound selected from the group consisting of (20S)-1,25-Dihydroxy-20-((2Z)-5,5,5-trifluoro-4-hydroxy-4-trifluoromethyl-pent-2-enyl)cholecalciferol (2); (20S)-1,25-Dihydroxy-20-[(2E)-5,5,5-trifluoro-4-hydroxy-4-trifluoromethyl-pent-2-enyl]-cholecalciferol (3); (20R)-1,25-Dihydroxy-20-(5,5,5-trifluoro-4-hydroxy-4-trifluoromethyl-pent-2-ynyl)-cholecalciferol (4); and (20R)-1,25-Dihydroxy-20-[(2E)-5,5,5-trifluoro-4-hydroxy-4-trifluoromethyl-pent-2-enyl]-cholecalciferol (5), thereby treating the subject for secondary hyperparathyroidism.

In yet another aspect, the invention provides a pharmaceutical composition for use in the treatment of osteroporosis, comprising a therapeutically effective amount of a vitamin D₃ compound selected from the group consisting of (20S)-1,25-Dihydroxy-20-(5,5,5-trifluoro-4-hydroxy-4-trifluoromethyl-pent-2-ynyl)-cholecalciferol (1); (20S)-1,25-Dihydroxy-20-[(2E)-5,5,5-trifluoro-4-hydroxy-4-trifluoromethyl-pent-2-enyl]-cholecalciferol (3); and (20S)-1α-Fluoro-25-hydroxy-20-(5,5,5-trifluoro-4-hydroxy-4-trifluoromethyl-pent-2-ynyl)-cholecalciferol (6), and a pharmaceutically acceptable diluent or carrier.

In still another aspect, the invention provides a pharmaceutical composition for use in the treatment of secondary hyperparathyroidism comprising a therapeutically effective amount of a vitamin D₃ compound selected from the group consisting of (20S)-1,25-Dihydroxy-20-((2Z)-5,5,5-trifluoro-4-hydroxy-4-trifluoromethyl-pent-2-enyl)cholecalciferol (2); (20S)-1,25-Dihydroxy-20-[(2E)-5,5,5-trifluoro-4-hydroxy-4-trifluoromethyl-pent-2-enyl]-cholecalciferol (3); (20R)-1,25-Dihydroxy-20-(5,5,5-trifluoro-4-hydroxy-4-trifluoromethyl-pent-2-ynyl)-cholecalciferol (4); and (20R)-1,25-Dihydroxy-20-[(2E)-5,5,5-trifluoro-4-hydroxy-4-trifluoromethyl-pent-2-enyl]-cholecalciferol (5), and a pharmaceutically acceptable diluent or carrier.

Another aspect of the invention provides a packaged formulation for use in the treatment of osteoporosis, comprising a pharmaceutical composition comprising a vitamin D₃ compound selected from the group consisting of (20S)-1,25-Dihydroxy-20-(5,5,5-trifluoro-4-hydroxy-4-trifluoromethyl-pent-2-ynyl)-cholecalciferol (1); (20S)-1,25-Dihydroxy-20-[(2E)-5,5,5-trifluoro-4-hydroxy-4-trifluoromethyl-pent-2-enyl]-cholecalciferol (3); and (20S)-1α-Fluoro-25-hydroxy-20-(5,5,5-trifluoro-4-hydroxy-4-trifluoromethyl-pent-2-ynyl)-cholecalciferol (6); and instructions for use in the treatment of osteoporosis.

Yet another aspect of the invention provides a packaged formulation for use in the treatment of secondary hyperparathyroidism, comprising a pharmaceutical composition comprising a vitamin D₃ compound selected from the group consisting of (20S)-1,25-Dihydroxy-20-((2Z)-5,5,5-trifluoro-4-hydroxy-4-trifluoromethyl-pent-2-enyl)cholecalciferol (2); (20S)-1,25-Dihydroxy-20-[(2E)-5,5,5-trifluoro-4-hydroxy-4-trifluoromethyl-pent-2-enyl]-cholecalciferol (3); (20R)-1,25-Dihydroxy-20-(5,5,5-trifluoro-4-hydroxy-4-trifluoromethyl-pent-2-ynyl)-cholecalciferol (4); and (20R)-1,25-Dihydroxy-20-[(2E)-5,5,5-trifluoro-4-hydroxy-4-trifluoromethyl-pent-2-enyl]-cholecalciferol (5), and instructions for use in the treatment of secondary hyperparathyroidism.

### BRIEF DESCRIPTION OF THE DRAWINGS

FIG. 1 shows tibia proximal metaphysic bone volume (μCT) measurements in 3 month old OVX rats.

FIG. 2 shows lumbar spine BMD (DEXA) measurements in 3 month old OVX rats.

FIG. 3 shows urinary calcium levels in 3 month old OVX rats.

FIG. 4 shows bone volume in 3 month old OVX rats using (1).

FIG. 5 shows a reevaluation of 3 month old OVX rats for tibia proximal metaphysic bone volume (ACT).

FIG. 6A shows serum Ca levels in 3 month old rats. FIG. 6B shows urinary Ca levels in 3 month old rats.

FIG. 7 shows travecular bone volume (ACT) measurements in 6 month old OVX rats.

FIG. 8 shows urinary calcium levels in 6 month old OVX rats.

FIG. 9 shows BMD) (DEXA) measurements in 6 month old OVX rats.

FIG. 11A shows serum calcium levels. FIG. 10B shows urinary calcium levels.

FIG. 11 shows BMD (DEXA) measurements in 6 month old OVX rats.

FIG. 12A shows parathyroid hormone (PTH) levels in rats with moderate renal failure. FIG. 12B shows serum Ca levels in rats with moderate renal failure.

FIGS. 13A and 13B show a model of safety parameters, measuring serum and serum Ca levels.

FIG. 14A shows a decrease in PTH levels in rats with severe chronic renal failure. FIG. 14B shows the serum calcium levels in rats with severe chronic renal failure.

FIGS. 15A and 15B show measurements of serum and serum Ca to determine safety profiles in rats with severe renal failure.

FIG. 16A shows the trabecular bone volume measurements in uremic rats.

FIG. 16B shows bine minarl density (pQCT) measurements in uremic rats.

FIGS. 17A, 17B, and 17C show tibia histomorphometry analysis, measuring bone formation rate, osteoblast surface, and osteoclast number in uremic rats with moderate renal failure.

FIG. 18A is a picture of a rat tibia using optical microscopy (×50) of a normal trabeculae. FIG. 18B is a picture of a rat tibia using optical microscopy (×50) of osteoid thickening. FIG. 18C is a picture of a rat tibia using optical microscopy (×50) of peritrabecular fibrosis.

FIG. 19 shows bone mineral density (DEXA) in uremic rats.

FIG. 20 is a picture of a rat femur cortical porosity using fluorescence microscopy (×8), showing normal porosity, mild porosity, medium porosity, and marked porosity.

FIG. 21 is a picture of a cross section of a rat aorta using Von Kossa staining (×100), showing a control, moderate aorta calcification, and severe aortic calcification.

### DETAILED DESCRIPTION OF THE INVENTION

### 1. Definitions

Before further description of the present invention, and in order that the invention may be more readily understood, certain terms are first defined and collected here for convenience.

The term “administration” or “administering” includes routes of introducing the vitamin D₃ compound(s) to a subject to perform their intended function. Examples of routes of administration which can be used include injection (subcutaneous, intravenous, parenterally, intraperitoneally, intrathecal), oral, inhalation, rectal and transdermal. The pharmaceutical preparations are, of course, given by forms suitable for each administration route. For example, these preparations are administered in tablets or capsule form, by injection, inhalation, eye lotion, ointment, suppository, etc. administration by injection, infusion or inhalation; topical by lotion or ointment; and rectal by suppositories. Oral administration is preferred. The injection can be bolus or can be continuous infusion. Depending on the route of administration, the vitamin D₃ compound can be coated with or disposed in a selected material to protect it from natural conditions which may detrimentally effect its ability to perform its intended function, The vitamin D₃ compound can be administered alone, or in conjunction with either another agent as described above or with a pharmaceutically-acceptable carrier, or both. The vitamin D₃ compound can be administered prior to the administration of the other agent, simultaneously with the agent, or after the administration of the agent. Furthermore, the vitamin D₃ compound can also be administered in a proform which is converted into its active metabolite, or more active metabolite in vivo.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. The term alkyl further includes alkyl groups, which can further include oxygen, nitrogen, sulfur or phosphorous atoms replacing one or more carbons of the hydrocarbon backbone, e.g., oxygen, nitrogen, sulfur or phosphorous atoms. In preferred embodiments, a straight chain or branched chain alkyl has 30 or fewer carbon atoms in its backbone (e.g., C₁-C₃₀ for straight chain, C₃-C₃₀ for branched chain), preferably 26 or fewer, and more preferably 20 or fewer. Likewise, preferred cycloalkyls have from 3-10 carbon atoms in their ring structure, and more preferably have 3, 4, 5, 6 or 7 carbons in the ring structure.

Moreover, the term alkyl as used throughout the specification and claims is intended to include both “unsubstituted alkyls” and “substituted alkyls,” the latter of which refers to alkyl moieties having substituents replacing a hydrogen on one or more carbons of the hydrocarbon backbone. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl)-amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. It will be understood by those skilled in the art that the moieties substituted on the hydrocarbon chain can themselves be substituted, if appropriate, Cycloalkyls can be further substituted, e.g., with the substituents described above. An “alkylaryl” moiety is an alkyl substituted with an aryl (e.g., phenylmethyl (benzyl)). The term “alkyl” also includes unsaturated aliphatic groups analogous in length and possible substitution to the alkyls described above, but that contain at least one double or triple bond respectively.

Unless the number of carbons is otherwise specified, “lower alkyl” as used herein means an alkyl group, as defined above, but having from one to ten carbons, more preferably from one to six, and most preferably from one to four carbon atoms in its backbone structure, which may be straight or branched-chain. Examples of lower alkyl groups include methyl, ethyl, n-propyl, i-propyl, tert-butyl, hexyl, heptyl, octyl and so forth. In preferred embodiment, the term “lower alkyl” includes a straight chain alkyl having 4 or fewer carbon atoms in its backbone, e.g., C₁-C₄ alkyl. The terms “alkoxyalkyl,” “polyaminoalkyl” and “thioalkoxyalkyl” refer to alkyl groups, as described above, which further include oxygen, nitrogen or sulfur atoms replacing one or more carbons of the hydrocarbon backbone, e.g., oxygen, nitrogen or sulfur atoms.

The terms “alkenyl” and “alkynyl” refer to unsaturated aliphatic groups analogous in length and possible substitution to the alkyls described above, but that contain at least one double or triple bond, respectively. For example, the invention contemplates cyano and propargyl groups.

The term “aryl” as used herein, refers to the radical of aryl groups, including 5- and 6-membered single-ring aromatic groups that may include from zero to four heteroatoms, for example, benzene, pyrrole, furan, thiophene, imidazole, benzoxazole, benzothiazole, triazole, tetrazole, pyrazole, pyridine, pyrazine, pyridazine and pyrimidine, and the like. Aryl groups also include polycyclic fused aromatic groups such as naphthyl, quinolyl, indolyl, and the like. Those aryl groups having heteroatoms in the ring structure may also be referred to as “aryl heterocycles,” “heteroaryls” or “heteroaromatics.” The aromatic ring can be substituted at one or more ring positions with such substituents as described above, as for example, halogen, hydroxyl, alkoxy, alkylcarbonyloxy, aryl carbonyloxy, alkoxycarbonyloxy, aryloxycarbonyloxy, carboxylate, alkylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The language “biological activities” of vitamin D₃ includes all activities elicited by vitamin D)₃ compounds in a responsive cell. It includes genomic and non-genomic activities elicited by these compounds (Gniadecki R. and Calverley M. J. (1998) Pharmacology & Toxicology 82: 173-176; Bouillon, R. et al. (1995) Endocrinology Reviews 16(2):206-207; Norman A. W. et al. (1992) J. Steroid Biochem Mol. Biol 41:231-240; Baran D. T. et al. (1991) J. Bone Miner Res. 6:1269-1275; Caffrey J. M. and Farach-Carson M. C. (1989) J. Biol. Chem. 264:20265-20274; Nemere I. et al. (1984) Endocrinology 115:1476-1483).

The language “bone metabolism” includes direct or indirect effects in the formation or degeneration of bone structures, e.g., bone formation, bone resorption, etc., which may ultimately affect the concentrations in serum of calcium and phosphate. This term is also intended to include effects of compounds of the invention in bone cells, e.g., osteoclasts and osteoblasts, that may in turn result in bone formation and degeneration.

The language “calcium and phosphate homeostasis” refers to the careful balance of calcium and phosphate concentrations, intracellularly and extracellularly, triggered by fluctuations in the calcium and phosphate concentration in a cell, a tissue, an organ or a system. Fluctuations in calcium levels that result from direct or indirect responses to compounds of the invention are intended to be included by these terms.

The term “chiral” refers to molecules which have the property of non-superimposability of the mirror image partner, while the term “achiral” refers to molecules which are superimposable on their mirror image partner.

The term “diastereomers” refers to stereoisomers with two or more centers of dissymmetry and whose molecules are not mirror images of one another.

The term “deuteroalkyl” refers to alkyl groups in which one or more of the of the hydrogens has been replaced with deuterium.

The term “effective amount” includes an amount effective, at dosages and for periods of time necessary, to achieve the desired result, e.g., sufficient treat a vitamin D₃ associated state or to modulate ILT3 expression in a cell. An effective amount of vitamin D₃ compound may vary according to factors such as the disease state, age, and weight of the subject, and the ability of the vitamin D₃ compound to elicit a desired response in the subject. Dosage regimens may be adjusted to provide the optimum therapeutic response. An effective amount is also one in which any toxic or detrimental effects (e.g., side effects) of compound are outweighed by the therapeutically beneficial effects.

A therapeutically effective amount of vitamin D₃ compound (i.e., an effective dosage) may range from about 0.001 to 30 μg/kg body weight, preferably about 0.01 to 25 μg/kg body weight, more preferably about 0.1 to 20 μg/kg body weight, and even more preferably about 1 to 10 μg/kg, 2 to 9 μg/kg, 3 to 8 μg/kg, 4 to 7 μg/kg, or 5 to 6 μg/kg body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a vitamin D₃ compound can include a single treatment or, preferably, can include a series of treatments. In one example, a subject is treated with a vitamin D₃ compound in the range of between about 0.1 to 20 μg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of a vitamin D₃ compound used for treatment may increase or decrease over the course of a particular treatment.

The term “enantiomers” refers to two stereoisomers of a compound which are non-superimposable mirror images of one another. An equimolar mixture of two enantiomers is called a “racemic mixture” or a “racemate.”

The language “Gemini vitamin D₃ compounds” is intended to include vitamin D₃ compounds and analogs thereof having bis C20 side chains. Vitamin D₃ compounds are characterized by an “A” ring (monocycle) which is connected to a “B” ring (bicycle) which is connected to a side chain at carbon C20 of the side chain. The Gemini compounds of the invention have two side chains and are, therefore, conspicuously distinguishable from vitamin D₃ compounds having a single side chain. Candidate A and B rings for the Gemini compounds of the invention are disclosed in U.S. Pat. Nos. 6,559,138, 6,329,538, 6,331,642, 6,452,028, 6,492,353, 6,040,461, 6,030,963, 5,939,408, 5,872,113, 5,840,718, 5,612,328, 5,512,554, 5,451,574, 5,428,029, 5,145,846, and 4,225,525. Examples of Gemini compounds in accordance with the invention are disclosed in U.S. Pat. No. 6,030,962.

The term “halogen” designates —F, —Cl, —Br or —I.

The term “haloalkyl” is intended to include alkyl groups as defined above that are mono-, di- or polysubstituted by halogen, e.g., fluoromethyl and trifluoromethyl.

The term “hydroxyl” means —OH.

The term “heteroatom” as used herein means an atom of any element other than carbon or hydrogen. Preferred heteroatoms are nitrogen, oxygen, sulfur and phosphorus.

The term “homeostasis” is art-recognized to mean maintenance of static, or constant, conditions in an internal environment.

The language “hormone secretion” is art-recognized and includes activities of vitamin D₃ compounds that control the transcription and processing responsible for secretion of a given hormone e.g., a parathyroid hormone (PTH) of a vitamin D₃ responsive cell (Bouillon, R. et al. (1995) Endocrine Reviews 16(2):235-237).

The language “hypercalcemia” or “hypercalcemic activity” is intended to have its accepted clinical meaning, namely, increases in calcium serum levels that are manifested in a subject by the following side effects, depression of central and peripheral nervous system, muscular weakness, constipation, abdominal pain, lack of appetite and, depressed relaxation of the heart during diastole. Symptomatic manifestations of hypercalcemia are triggered by a stimulation of at least one of the following activities, intestinal calcium transport, bone calcium metabolism and osteocalcin synthesis (reviewed in Boullion, R. et al. (1995) Endocrinology Reviews 16(2): 200-257).

The language “improved biological properties” refers to any activity inherent in a compound of the invention that enhances its effectiveness in vivo. In a preferred embodiment, this term refers to any qualitative or quantitative improved therapeutic property of a vitamin D₃ compound, such as reduced toxicity, e.g., reduced hypercalcemic activity.

The term “isomers” or “stereoisomers” refers to compounds which have identical chemical constitution, but differ with regard to the arrangement of the atoms or groups in space.

The term “modulate” refers to increases or decreases in the activity of a cell in response to exposure to a compound of the invention, e.g., the inhibition of proliferation and/or induction of differentiation of at least a sub-population of cells in an animal such that a desired end result is achieved, e.g., a therapeutic result. In preferred embodiments, this phrase is intended to include hyperactive conditions that result in pathological disorders.

The language “non-genomic” vitamin D₃ activities include cellular (e.g., calcium transport across a tissue) and subcellular activities (e.g., membrane calcium transport opening of voltage-gated calcium channels, changes in intracellular second messengers) elicited by vitamin D₃ compounds in a responsive cell. Electrophysiological and biochemical techniques for detecting these activities are known in the art. An example of a particular well-studied non-genomic activity is the rapid hormonal stimulation of intestinal calcium mobilization, termed “transcaltachia” (Nemere I. et al. (1984) Endocrinology 115:1476-1483; Lieberherr M. et al. (1989) J. Biol. Chem. 264:20403-20406; Wali R. K. et al. (1992) Endocrinology 131:1125-1133; Wali R. K et al. (1992) Am. J. Physiol. 262:G945-G953; Wali R. K. et al. (1990) J. Chem. Invest. 85:1296-1303; Bolt M. J. G. et al. (1993) Biochem. J. 292:271-276). Detailed descriptions of experimental transcaltachia are provided in Norman, A. W. (1993) Endocrinology 268(27):20022-20030; Yoshimoto, Y. and Norman, A. W. (1986) Endocrinology 118:2300-2304. Changes in calcium activity and second messenger systems are well known in the art and are extensively reviewed in Bouillon, R. et al. (1995) Endocrinology Review 16(2): 200-257; the description of which is incorporated herein by reference.

The term “obtaining” as in “obtaining a vitamin D₃ compound” is intended to include purchasing, synthesizing or otherwise acquiring the compound.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The terms “polycyclyl” or “polycyclic radical” refer to the radical of two or more cyclic rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, sulfonato, sulfamoyl, sulfonamido, nitro, trifluoromethyl, cyano, azido, heterocyclyl, alkyl, alkylaryl, or an aromatic or heteroaromatic moiety.

The term “prodrug” includes compounds with moieties which can be metabolized in vivo. Generally, the prodrugs are metabolized in vivo by esterases or by other mechanisms to active drugs. Examples of prodrugs and their uses are well known in the art (See, e.g. Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Preferred prodrug moieties are propionic acid esters and acyl esters. Prodrugs which are converted to active forms through other mechanisms in vivo are also included.

The language “reduced toxicity” is intended to include a reduction in any undesired side effect elicited by a vitamin D₃ compound when administered in vivo, e.g., a reduction in the hypercalcemic activity.

The term “secosteroid” is art-recognized and includes compounds in which one of the cyclopentanoperhydro-phenanthrene rings of the steroid ring structure is broken. 1α,25(OH)₂D₃ and analogs thereof are hormonally active secosteroids. In the case of vitamin D₃, the 9-10 carbon-carbon bond of the B-ring is broken, generating a seco-B-steroid. The official IUPAC name for vitamin D₃ is 9,10-secocholesta-5,7,10(19)-trien-3B-ol. For convenience, a 6-s-trans conformer of 1α,25(OH)₂D₃ is illustrated herein having all carbon atoms numbered using standard steroid notation.

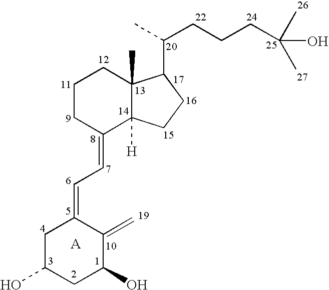

In the formulas presented herein, the various substituents on ring A are illustrated as joined to the steroid nucleus by one of these notations: a dotted line ( - - - ) indicating a substituent which is in the β-orientation (i.e., above the plane of the ring), a wedged solid line ( [symbol] ) indicating a substituent which is in the α-orientation (i.e., below the plane of the molecule), or a wavy line ( [symbol] ) indicating that a substituent may be either above or below the plane of the ring. In regard to ring A, it should be understood that the stereochemical convention in the vitamin D field is opposite from the general chemical field, wherein a dotted line indicates a substituent on Ring A which is in an α-orientation (i.e., below the plane of the molecule), and a wedged solid line indicates a substituent on ring A which is in the β-orientation (i.e., above the plane of the ring). As shown, the A ring of the hormone 1α,25(OH)₂D₃ contains two asymmetric centers at carbons 1 and 3, each one containing a hydroxyl group in well-characterized configurations, namely the 1α- and 3β-hydroxyl groups. In other words, carbons 1 and 3 of the A ring are said to be “chiral carbons” or “carbon centers”.

Also, throughout the patent literature, the A ring of a vitamin D compound is often depicted in generic formulae as any one of the following structures:

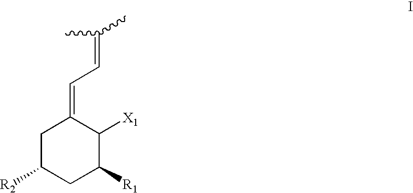

wherein X₁ is defined as H (or H₂) or ═CH₂; or

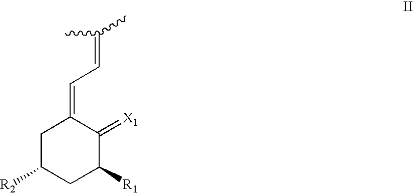

wherein X₁ is defined as H₂ or CH₂. Although there does not appear to be any set convention, it is clear that one of ordinary skill in the art understands either formula I or II to represent an A ring in which, for example, X₁ is ═CF₂, as follows:

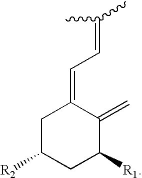

For purposes of the instant invention, the representation of the A ring as shown immediately above in formula II will be used in all generic structures.

Furthermore the indication of stereochemistry across a carbon-carbon double bond is also opposite from the general chemical field in that “Z” refers to what is often referred to as a “cis” (same side) conformation whereas “E” refers to what is often referred to as a “trans” (opposite side) conformation. As shown, the A ring of the hormone 1-alpha,25(OH)₂D₃ contains two asymmetric centers at carbons 1 and 3, each one containing a hydroxyl group in well-characterized configurations, namely the 1-alpha- and 3-beta-hydroxyl groups. In other words, carbons 1 and 3 of the A ring are said to be “chiral carbons” or “chiral carbon centers.” Regardless, both configurations, cis/trans and/or Z/E are encompassed by the compounds of the present invention. With respect to the nomenclature of a chiral center, the terms “d” and “1” configuration are as defined by the IUPAC Recommendations. As to the use of the terms, diastereomer, racemate, epimer and enantiomer, these will be used in their normal context to describe the stereochemistry of preparations.

The term “subject” includes organisms which are capable of suffering from a vitamin D₃ associated state or who could other wise benefit from the administration of a vitamin D₃ compound of the invention, such as human and non-human animals. Preferred human animals include human patients suffering from or prone to suffering from a vitamin D₃ associated state, as described herein. The term “non-human animals” of the invention includes all vertebrates, e.g., mammals, e.g., rodents, e.g., mice, and non-mammals, such as non-human primates, sheep, dog, cow, chickens, amphibians, reptiles, etc.

The term “sulfhydryl” or “thiol” means —SH.

The phrases “systemic administration” “administered systemically”, “peripheral administration” and “administered peripherally” as used herein mean the administration of a vitamin D₃ compound(s), drug or other material, such that it enters the patient's system and, thus, is subject to metabolism and other like processes, for example, subcutaneous administration.

The term “VDR” is intended to include members of the type II class of steroid/thyroid superfamily of receptors (Stunnenberg, H. G. (1993) Bio Essays 15(5):309-15), which are able to bind and transactivate through the vitamin D response element (VDRE) in the absence of a ligand (Damm et al. (1989) Nature 339:593-97; Sap et al. Nature 343:177-180).

The term “VDRE” refers to DNA sequences composed of half-sites arranged as direct repeats. It is known in the art that type II receptors do not bind to their respective binding site as homodimers but require an auxiliary factor, RXR (e.g. RXRα, RXRβ, RXRγ) for high affinity binding Yu et al., (1991) Cell 67:1251-1266; Bugge et al. (1992) EMBO J. 11:1409-1418; Kliewer et al. (1992) Nature 355:446-449; Leid et al. (1992), EMBO J. 11:1419-1435; Zhang et al. (1992) Nature 355:441-446).

The language “vitamin D₃ associated state” is a state which can be prevented, treated or otherwise ameliorated by administration of one or more compounds of the invention. Vitamin D₃ associated states include ILT3-associated disorders, disorders characterized by an aberrant activity of a vitamin D₃-responsive cell, disorders characterized by a deregulation of calcium and phosphate metabolism, and other disorders or states described herein.

The term “vitamin D₃-responsive cell” includes any cell which is capable of responding to a vitamin D₃ compound described herein, or is associated with disorders involving an aberrant activity of hyperproliferative skin cells, parathyroid cells, neoplastic cells, immune cells, and bone cells. These cells can respond to vitamin D₃ activation by triggering genomic and/or non-genomic responses that ultimately result in the modulation of cell proliferation, differentiation survival, and/or other cellular activities such as hormone secretion. In a preferred embodiment, the ultimate responses of a cell are inhibition of cell proliferation and/or induction of differentiation-specific genes. Exemplary vitamin D₃ responsive cells include immune cells, bone cells, neuronal cells, endocrine cells, neoplastic cells, epidermal cells, endodermal cells, smooth muscle cells, among others.

With respect to the nomenclature of a chiral center, terms “d” and “1” configuration are as defined by the IUPAC Recommendations. As to the use of the terms, diastereomer, racemate, epimer and enantiomer will be used in their normal context to describe the stereochemistry of preparations.

### 2. Gemini Vitamin D₃ Compounds

In certain aspects, the invention provides for the use of vitamin D compounds to treat osteoporosis and secondary hyperparathyroidism. Preferred compounds for use in the methods of the invention include the following compounds:

### (20S)-1,25-Dihydroxy-20-(5,5,5-trifluoro-4-hydroxy-4-trifluoromethyl-pent-2-ynyl)-cholecalciferol (1)

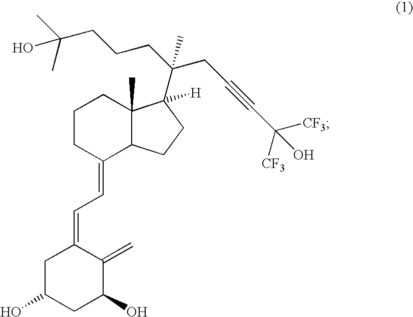

### (20S)-1,25-Dihydroxy-20-((2Z)-5,5,5-trifluoro-4-hydroxy-4-trifluoromethyl-pent-2-enyl)cholecalciferol (2)

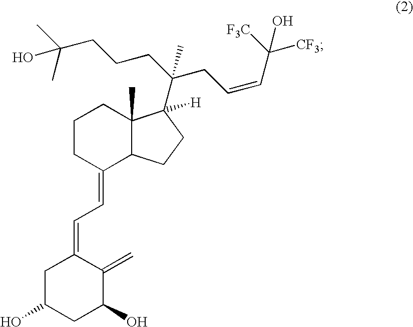

### (20S)-1,25-Dihydroxy-20-[(2E)-5,5,5-trifluoro-4-hydroxy-4-trifluoromethyl-pent-2-enyl]-cholecalciferol (3)

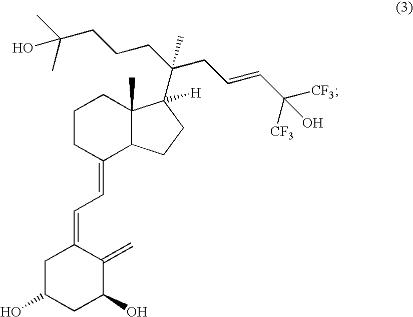

### (20R)-1,25-Dihydroxy-20-(5,5,5-trifluoro-4-hydroxy-4-trifluoromethyl-pent-2-ynyl)-cholecalciferol (4)

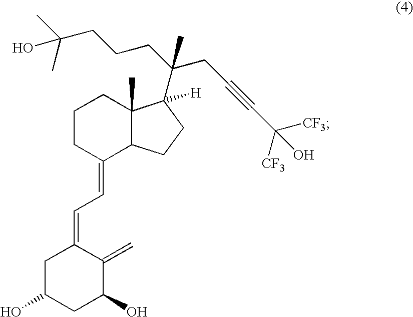

### (20R)-1,25-Dihydroxy-20-[(2E)-5,5,5-trifluoro-4-hydroxy-4-trifluoromethyl-pent-2-enyl]-cholecalciferol (5)

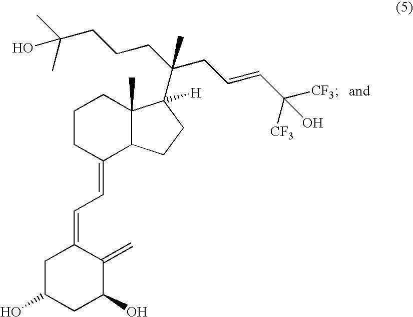

### (20S)-1α-Fluoro-25-hydroxy-20-(5,5,5-trifluoro-4-hydroxy-4-trifluoromethyl-pent-2-ynyl)-cholecalciferol (6)

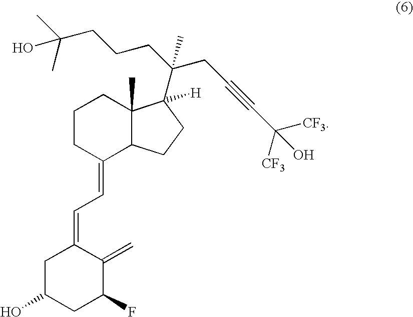

In certain embodiments, especially preferred compounds include (20S)-1,25-Dihydroxy-20-(5,5,5-trifluoro-4-hydroxy-4-trifluoromethyl-pent-2-ynyl)-cholecalciferol (1) and (20S)-1,25-Dihydroxy-20-((2Z)-5,5,5-trifluoro-4-hydroxy-4-trifluoromethyl-pent-2-enyl)cholecalciferol (2).

The structures of some of the compounds of the invention include asymmetric carbon atoms. Accordingly, the isomers arising from such asymmetry (e.g., all enantiomers and diastereomers) are included within the scope of this invention, unless indicated otherwise. Such isomers can be obtained in substantially pure form by classical separation techniques and/or by stereochemically controlled synthesis.

Naturally occurring or synthetic isomers can be separated in several ways known in the art. Methods for separating a racemic mixture of two enantiomers include chromatography using a chiral stationary phase (see, e.g., “Chiral Liquid Chromatography,” W. J. Lough, Ed. Chapman and Hall, New York (1989)). Enantiomers can also be separated by classical resolution techniques. For example, formation of diastereomeric salts and fractional crystallization can be used to separate enantiomers. For the separation of enantiomers of carboxylic acids, the diastereomeric salts can be formed by addition of enantiomerically pure chiral bases such as brucine, quinine, ephedrine, strychnine, and the like. Alternatively, diastereomeric esters can be formed with enantiomerically pure chiral alcohols such as menthol, followed by separation of the diastereomeric esters and hydrolysis to yield the free, enantiomerically enriched carboxylic acid. For separation of the optical isomers of amino compounds, addition of chiral carboxylic or sulfonic acids, such as camphorsulfonic acid, tartaric acid, mandelic acid, or lactic acid can result in formation of the diastereomeric salts.

### 3. Uses of the Vitamin D₃ Compounds of the Invention

In current methods, the use of vitamin D₃ compounds has been limited because of their hypercalcemic effects. The 20-methyl Gemini vitamin D₃ compounds of the invention can provide a less toxic alternative to current methods of treatment for osteoporosis and secondary hyperparathyroidism.

In one aspect, the invention provides a method for treating osteoporosis in a subject comprising administering to a subject in need thereof a therapeurically effective amount of a 20-methyl Gemini vitamin D₃ compound of the invention, thereby treating said subject for osteoporosis. Preferred compounds for this aspect of the invention include (20S)-1,25-Dihydroxy-20-(5,5,5-trifluoro-4-hydroxy-4-trifluoromethyl-pent-2-ynyl)-cholecalciferol (1); (20S)-1,25-Dihydroxy-20-[(2E)-5,5,5-trifluoro-4-hydroxy-4-trifluoromethyl-pent-2-enyl]-cholecalciferol (3); and (20S)-1α-Fluoro-25-hydroxy-20-(5,5,5-trifluoro-4-hydroxy-4-trifluoromethyl-pent-2-ynyl)-cholecalciferol (6). An especially preferred compound of the invention is (20S)-1,25-Dihydroxy-20-(5,5,5-trifluoro-4-hydroxy-4-trifluoromethyl-pent-2-ynyl)-cholecalciferol (1).

In one embodiment of this aspect of the invention, the method further comprises identifying a subject as being in need of treatment for osteoporosis. In another embodiment the method further comprises obtaining the vitamin D₃ compound.

Another aspect of the invention provides a method for treating a subject for secondary hyperparathyroidism comprising administering to a subject in need thereof a therapeutically effective amount of a 20-methyl Gemini vitamin D₃ compound of the invention, thereby treating the subject for secondary hyperparathyroidism. Preferred compounds for this aspect of the invention include (20S)-1,25-Dihydroxy-20-((2Z)-5,5,5-trifluoro-4-hydroxy-4-trifluoromethyl-pent-2-enyl)cholecalciferol (2); (20S)-1,25-Dihydroxy-20-[(2E)-5,5,5-trifluoro-4-hydroxy-4-trifluoromethyl-pent-2-enyl]-cholecalciferol (3); (20R)-1,25-Dihydroxy-20-(5,5,5-trifluoro-4-hydroxy-4-trifluoromethyl-pent-2-ynyl)-cholecalciferol (4); and (20R)-1,25-Dihydroxy-20-[(2E)-5,5,5-trifluoro-4-hydroxy-4-trifluoromethyl-pent-2-enyl]-cholecalciferal (5). An especially preferred compound of the invention is (20S)-1,25-Dihydroxy-20-((2Z)-5,5,5-trifluoro-4-hydroxy-4-trifluoromethyl-pent-2-enyl)cholecalciferol (Z).

In one embodiment of this aspect of the invention, the method further comprises identifying a subject as being in need of treatment for secondary hyperparathyroidism. In another embodiment the method further comprises obtaining the vitamin D₃ compound.

In certain embodiments of the methods of the invention, the subject is a mammal. In preferred embodiments, the subject is human.

In one embodiment, the vitamin D₃ compound is administered to the subject using a pharmaceutically-acceptable formulation. In certain embodiments, the vitamin D₃ compound is advantageously administered in combination with a pharmaceutically acceptable diluent or carrier.

In another embodiment) the pharmaceutically-acceptable formulation provides sustained delivery of the vitamin D₃ compound to a subject for at least four weeks after the pharmaceutically-acceptable formulation is administered to the subject.

In accordance with the methods of the invention, the vitamin D₃ compound is administered orally, intravenously, topically, or parenterally. Although dosages may vary depending on the particular indication, route of administration and subject, the 20-methyl Gemini vitamin D₃ compounds of the invention are administered at a concentration of about 0.001 μg to about 100 μg/kg of body weight. In certain embodiments, the 20-methyl Gemini vitamin D₃ compounds of the invention are administered at a concentration of about 5 μg/kg of body weight.

Another aspect of the invention provides a pharmaceutical composition for use in the treatment of osteoporosis, comprising a therapeutically effective amount of a 20-methyl Gemini vitamin D₃ compound of the invention and a pharmaceutically acceptable diluent or carrier. Preferred compounds of this aspect of the invention include (20S)-1,25-Dihydroxy-20-(5,5,5-trifluoro-4-hydroxy-4-trifluoromethyl-pent-2-ynyl)-cholecalciferol (1); (20S)-1,25-Dihydroxy-20-[(2E)-5,5,5-trifluoro-4-hydroxy-4-trifluoromethyl-pent-2-enyl]-cholecalciferol (3); and (20S)-1α-Fluoro-25-hydroxy-20-(5,5,5-trifluoro-4-hydroxy-4-trifluoromethyl-pent-2-ynyl)-cholecalciferol (6). An especially preferred compound of the invention is (20S)-1,25-Dihydroxy-20-(5,5,5-trifluoro-4-hydroxy-4-trifluoromethyl-pent-2-ynyl)-cholecalciferol (1).

In yet another aspect, the invention provides a pharmaceutical composition for use in the treatment of secondary hyperparathyroidism comprising a therapeutically effective amount of a 20-methyl Gemini vitamin D₃ compound of the invention and a pharmaceutically acceptable diluent or carrier. Preferred compounds of this aspect of the invention include (20S)-1,25-Dihydroxy-20-((2Z)-5,5,5-trifluoro-4-hydroxy-4-trifluoromethyl-pent-2-enyl)cholecalciferol (2); (20S)-1,25-Dihydroxy-20-[(2E)-5,5,5-trifluoro-4-hydroxy-4-trifluoromethyl-pent-2-enyl]-cholecalciferol (3); (20R)-1,25-Dihydroxy-20-(5,5,5-trifluoro-4-hydroxy-4-trifluoromethyl-pent-2-ynyl)-cholecalciferol (4); and (20R)-1,25-Dihydroxy-20-[(2E)-5,5,5-trifluoro-4-hydroxy-4-trifluoromethyl-pent-2-enyl]-cholecalciferol (5). A particularly preferred compound is (20S)-1,25-Dihydroxy-20-((27)-5,5,5-trifluoro-4-hydroxy-4-trifluoromethyl-pent-2-enyl)cholecalciferol (2).

In another aspect, the invention provides a packaged formulation for use in the treatment of osteoporosis, comprising a pharmaceutical composition comprising a 20-methyl Gemini vitamin D₃ compound and instructions for use in the treatment of osteoporosis in accordance with the methods of the invention. Preferred compounds of this aspect of the invention include (20S)-1,25-Dihydroxy-20-(5,5,5-trifluoro-4-hydroxy-4-trifluoromethyl-pent-2-ynyl)-cholecalciferol (1); (20S)-1,25-Dihydroxy-20-[(2E)-5,5,5-trifluoro-4-hydroxy-4-trifluoromethyl-pent-2-enyl]-cholecalciferol (3); and (20S)-1α-Fluoro-25-hydroxy-20-(5,5,5-trifluoro-4-hydroxy-4-trifluoromethyl-pent-2-ynyl)-cholecalciferol (6). An especially preferred compound of the invention is (20S)-1,25-Dihydroxy-20-(5,5,5-trifluoro-4-hydroxy-4-trifluoromethyl-pent-2-ynyl)-cholecalciferol (1).

Yet another aspect of the invention provides a packaged formulation for use in the treatment of secondary hyperparathyroidism, comprising a pharmaceutical composition comprising a 20-methyl Gemini vitamin D₃ compound and instructions for use in the treatment of secondary hyperparathyroidism in accordance with the methods of the invention. Preferred compounds of this aspect of the invention include (20S)-1,25-Dihydroxy-20-((2Z)-5,5,5-trifluoro-4-hydroxy-4-trifluoromethyl-pent-2-enyl)cholecalciferol (2); (20S)-1,25-Dihydroxy-20-[(2E)-5,5,5-trifluoro-4-hydroxy-4-trifluoromethyl-pent-2-enyl]-cholecalciferol (3); (20R)-1,25-Dihydroxy-20-(5,5,5-trifluoro-4-hydroxy-4-trifluoromethyl-pent-2-ynyl)-cholecalciferol (4); and (20R)-1,25-Dihydroxy-20-[(2E)-5,5,5-trifluoro-4-hydroxy-4-trifluoromethyl-pet-2-enyl]-cholecalciferol (5). A particularly preferred compound is (20S)-1,25-Dihydroxy-20-((2Z)-5,5,5-trifluoro-4-hydroxy-4-trifluoromethyl-pent-2-enyl)cholecalciferol (2).

### 4. Pharmaceutical Compositions

The invention also provides a pharmaceutical composition, comprising an effective amount a vitamin D₃ compound described herein and a pharmaceutically acceptable carrier. In a further embodiment, the effective amount is effective to treat a vitamin D₃ associated state, as described previously.

In an embodiment, the vitamin D₃ compound is administered to the subject using a pharmaceutically-acceptable formulation, e.g., a pharmaceutically-acceptable formulation that provides sustained delivery of the vitamin D₃ compound to a subject for at least 12 hours, 24 hours, 36 hours, 48 hours, one week, two weeks, three weeks, or four weeks after the pharmaceutically-acceptable formulation is administered to the subject.

In certain embodiments, these pharmaceutical compositions are suitable for topical or oral administration to a subject. In other embodiments, as described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

In certain embodiments, the subject is a mammal, e.g., a primate, e.g., a human.

The methods of the invention further include administering to a subject a therapeutically effective amount of a vitamin D₃ compound in combination with another pharmaceutically active compound. Examples of pharmaceutically active compounds include compounds known to treat autoimmune disorders, e.g., immunosuppressant agents such as cyclosporin A, rapamycin, desoxyspergualine, FK 506, steroids, azathioprine, anti-T cell antibodies and monoclonal antibodies to T cell subpopulations. Other pharmaceutically active compounds that may be used can be found in Harrison's Principles of Internal Medicine, Thirteenth Edition, Eds. T. R. Harrison et al. McGraw-Hill N.Y., NY; and the Physicians Desk Reference 50th Edition 1997, Oradell N.J., Medical Economics Co., the complete contents of which are expressly incorporated herein by reference. The angiogenesis inhibitor compound and the pharmaceutically active compound may be administered to the subject in the same pharmaceutical composition or in different pharmaceutical compositions (at the same time or at different times).

The phrase “pharmaceutically acceptable” is refers to those vitamin 3 compounds of the present invention, compositions containing such compounds, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” includes pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Compositions containing a vitamin D₃ compound(s) include those suitable for oral, nasal, topical (including buccal and sublingual), rectal, vaginal, aerosol and/or parenteral administration. The compositions may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these compositions include the step of bringing into association a vitamin D₃ compound(s) with the carrier and, optionally, one or more accessory ingredients. In general, the formulations are prepared by uniformly and intimately bringing into association a vitamin D₃ compound with liquid carriers, or finely divided solid carriers, or both, and then, if necessary, shaping the product.

Compositions of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a vitamin D₃ compound(s) as an active ingredient. A compound may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered active ingredient moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or sonic other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the vitamin D₃ compound(s) include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

In addition to inert diluents, the oral compositions can include adjuvants such as wetting agents, emulsifying and suspending agents, sweetening, flavoring, coloring, perfuming and preservative agents.

Suspensions, in addition to the active vitamin D₃ compound(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of the invention for rectal or vaginal administration may be presented as a suppository, which may be prepared by mixing one or more vitamin D₃ compound(s) with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Compositions of the present invention which are suitable for vaginal administration also include pessaries, tampons, creams, gels, pastes, foams or spray formulations containing such carriers as are known in the art to be appropriate.

Dosage forms for the topical or transdermal administration of a vitamin D₃ compound(s) include powders, sprays, ointments, pastes, creams, lotions, gels, solutions, patches and inhalants. The active vitamin D₃ compound(s) may be mixed under sterile conditions with a pharmaceutically-acceptable carrier, and with any preservatives, buffers, or propellants which may be required.

The ointments, pastes, creams and gels may contain, in addition to vitamin D₃ compound(s) of the present invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a vitamin D₃ compound(s), excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The vitamin D₃ compound(s) can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, all aqueous aerosol is made by formulating an aqueous solution or suspension of the agent together with conventional pharmaceutically-acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of a vitamin D₃ compound(s) to the body. Such dosage forms can be made by dissolving or dispersing the agent in the proper medium. Absorption enhancers can also be used to increase the flux of the active ingredient across the skin. The rate of such flux can be controlled by either providing a rate controlling membrane or dispersing the active ingredient in a polymer matrix or gel.

Ophthalmic formulations, eye ointments, powders, solutions and the like, are also contemplated as being within the scope of this invention.

Pharmaceutical compositions of this invention suitable for parenteral administration comprise one or more vitamin D₃ compound(s) in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of vitamin D₃ compound(s) in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the vitamin D₃ compound(s) are administered as pharmaceuticals, to humans and animals, they can be given per se or as a pharmaceutical composition containing, for example, 0.1 to 99.5% (more preferably, 0.5 to 90%) of active ingredient in combination with a pharmaceutically-acceptable carrier.

Regardless of the route of administration selected, the vitamin D₃ compound(s), which may be used in a suitable hydrated form, and/or the pharmaceutical compositions of the present invention, are formulated into pharmaceutically-acceptable dosage forms by conventional methods known to those of skill in the art.

Actual dosage levels and time course of administration of the active ingredients in the pharmaceutical compositions of this invention may be varied so as to obtain an amount of the active ingredient which is effective to achieve the desired therapeutic response for a particular patient, composition, and mode of administration, without being toxic to the patient. An exemplary dose range is from 0.1 to 10 mg per day.

A preferred dose of the vitamin D₃ compound for the present invention is the maximum that a patient can tolerate and not develop serious hypercalcemia. Preferably, the vitamin D₃ compound of the present invention is administered at a concentration of about 0.001 μg to about 100 μg per kilogram of body weight, about 0.001-about 10 μg/kg or about 0.001 μg-about 100 μg/kg of body weight. Ranges intermediate to the above-recited values are also intended to be part of the invention.

### 5. Synthesis of Compounds of the Invention

Compounds of the invention can be synthesized by methods described in this section, the examples, and the chemical literature.

Schemes 1-9 below depict the reaction steps for the synthesis of the highly fluorinated-20-methyl gemini vitamin D₃ compounds of the invention.

Scheme 1 shows the synthetic route for the production of the diol 15 and its epimer, 16. Alcohol 7 was protected with a silyl group to compound 8, then cyclopropanated to provide cyclopropane 9. Conversion of the ester to the aldehyde was accomplished over two steps to provide 11. Chain elongation using a modified Wittig-Horner reaction provided 12. Reduction of the double bond and cyclopropane opening liberated ester 13, which was reduced to alcohol 14. Deprotection and chromatographic separation yielded intermediate 15 and its epimer 16.

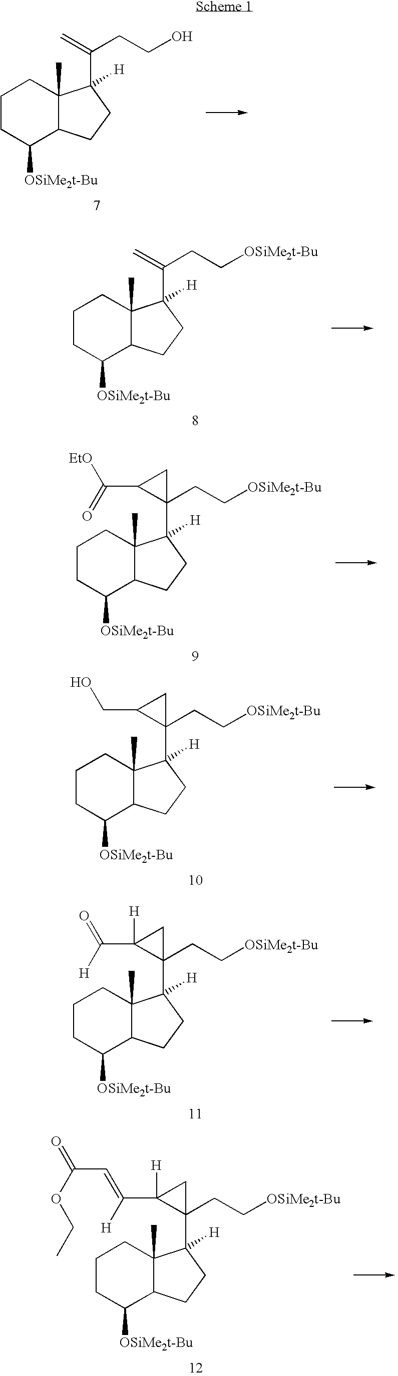

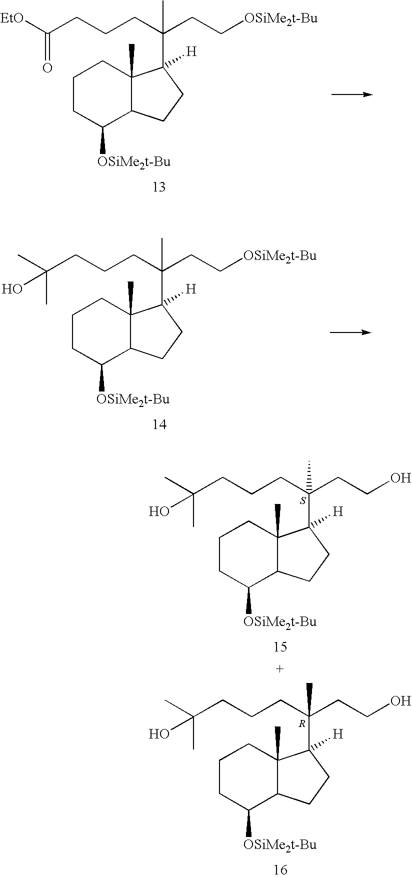

Scheme 2 shows the chain elongation of 15 to triol 21. Oxidation of the primary alcohol of 15 provided the corresponding aldehyde 17 and chain elongation provided alkyne 18. Protection of the tertiary alcohol to compound 19 was followed by lithiation of the alkyne and reaction with hexafluoroacetone to produce 20 Silyl group deprotection provided triol 21.

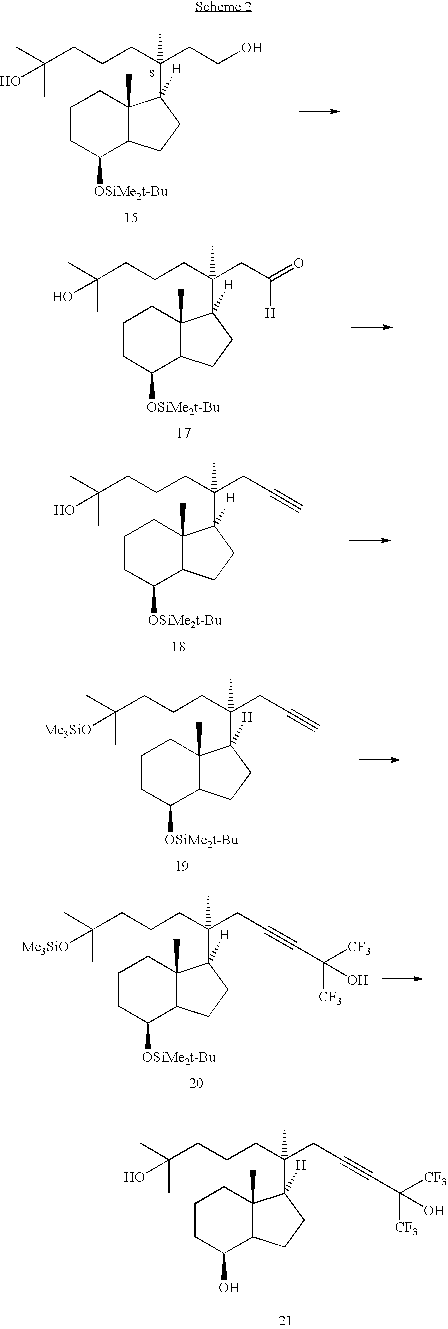

Scheme 3 shows that 21 is oxidized to ketone 22 which is amenable to Wittig-Horner coupling with an appropriate phosphine oxide. Further reduction of the alkyne of 21 was carried out to form either the cis or trans olefins, 23 and 25, respectively. Oxidation provided ketones 24 and 26.

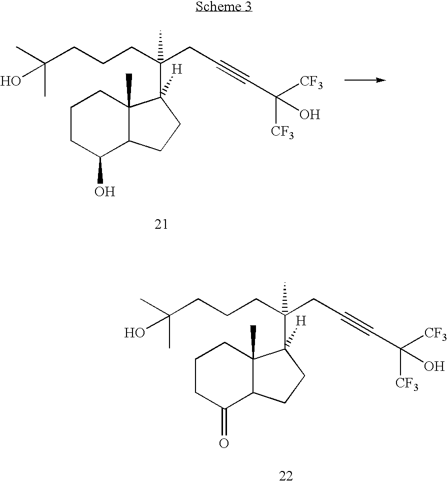

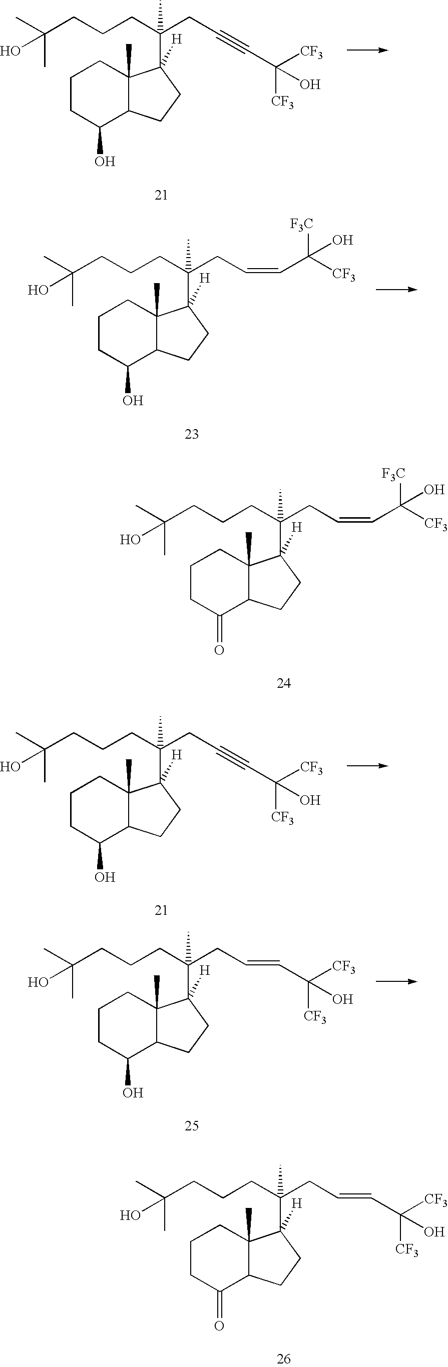

Scheme 4 shows the Wittig-Horner coupling of ketone 22 with phosphine oxide 27 in the presence of base to provide the corresponding coupled product. Scheme 4 also shows the coupling of hydroxyl protected ketone 28 with phosphine oxide 29 to provide the corresponding coupled product. Deprotection of the silyl group(s) with tetrabutyl ammonium fluoride afforded compounds 1 and 6.

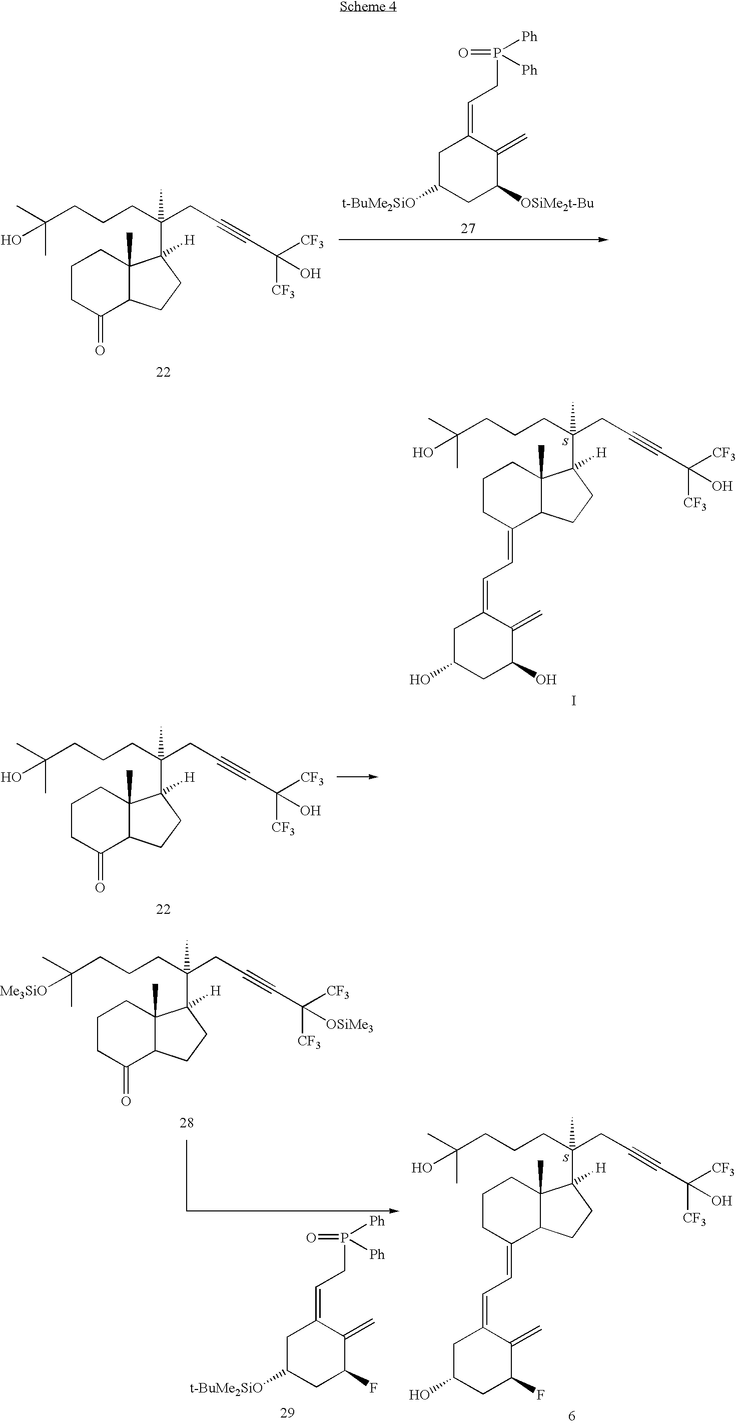

Scheme 5 shows the Wittig-Horner coupling of ketone 24 with phosphine oxide 27 in the presence of base to provide the corresponding coupled product. Deprotection of the silyl group(s) with tetrabutyl ammonium fluoride afforded compound 2.

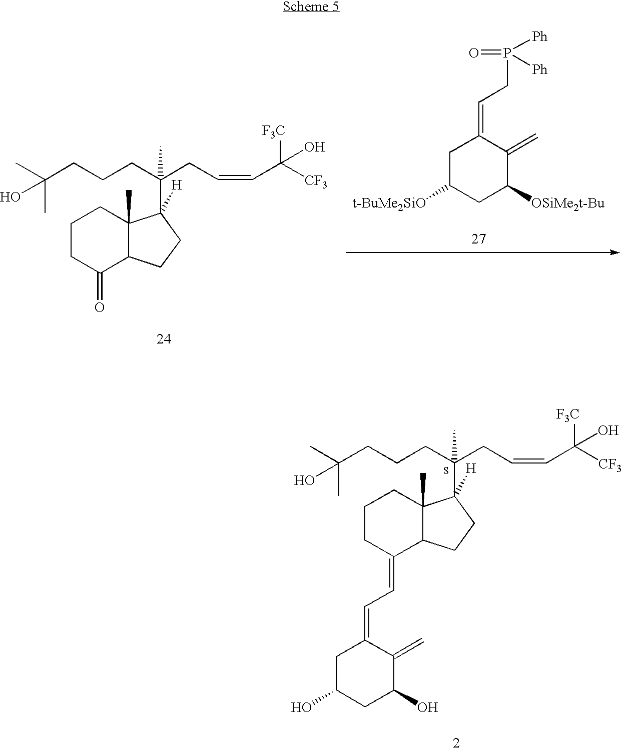

Scheme 6 shows the Wittig-Horner coupling of ketone 26 with phosphine oxide 27 in the presence of base to provide the corresponding coupled product. Deprotection of the silyl group(s) with tetrabutyl ammonium fluoride afforded compound 3.

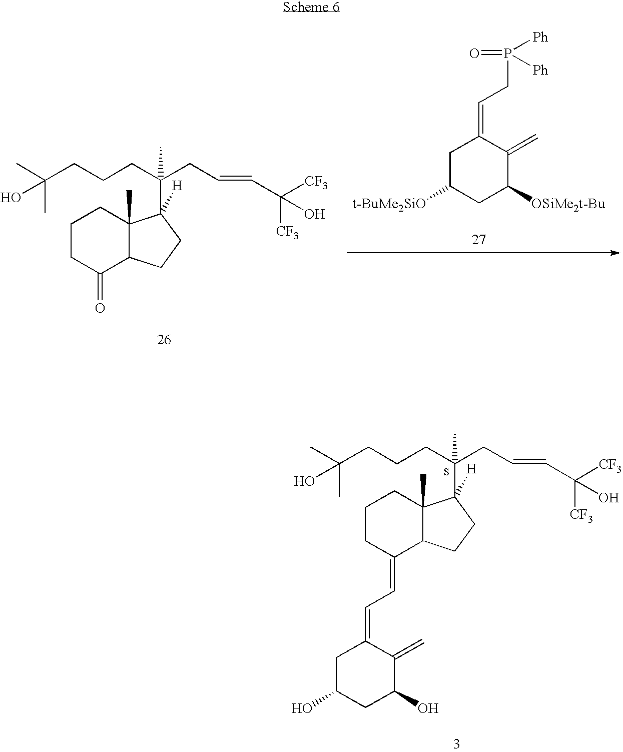

Vitamin D₃ compounds 4 and 5 were synthesized in accordance with the reactions described in schemes 1-6 above. The starting material for the production of 4 and 5 was 16. Scheme 7 shows the chain elongation of 16 to triol 34. Oxidation of the primary alcohol of 16 provided the corresponding aldehyde and chain elongation provided alkyne 31. Protection of the tertiary alcohol was followed by lithiation of the alkyne and reaction with hexafluoroacetone to produce 33. Silyl group deprotection provided triol 34.

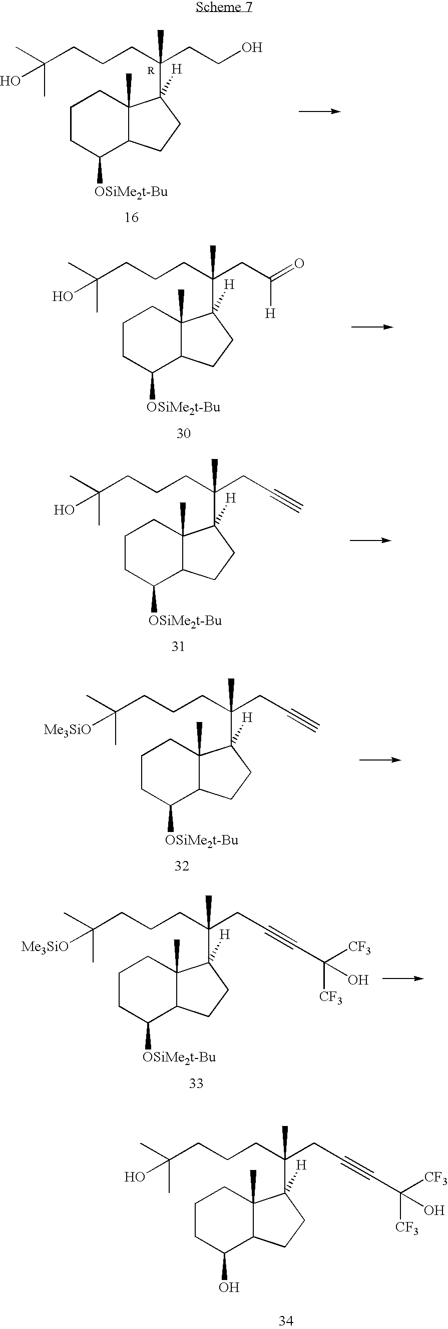

Scheme 8 shows that 34 is oxidized to foim ketone 35 that is amenable to Wittig-Horner coupling with an appropriate phosphine oxide. Further reduction of the alkyne of 34 was carried out to form the trans olefin 36. Oxidation and hydroxyl protection provided ketone 38.

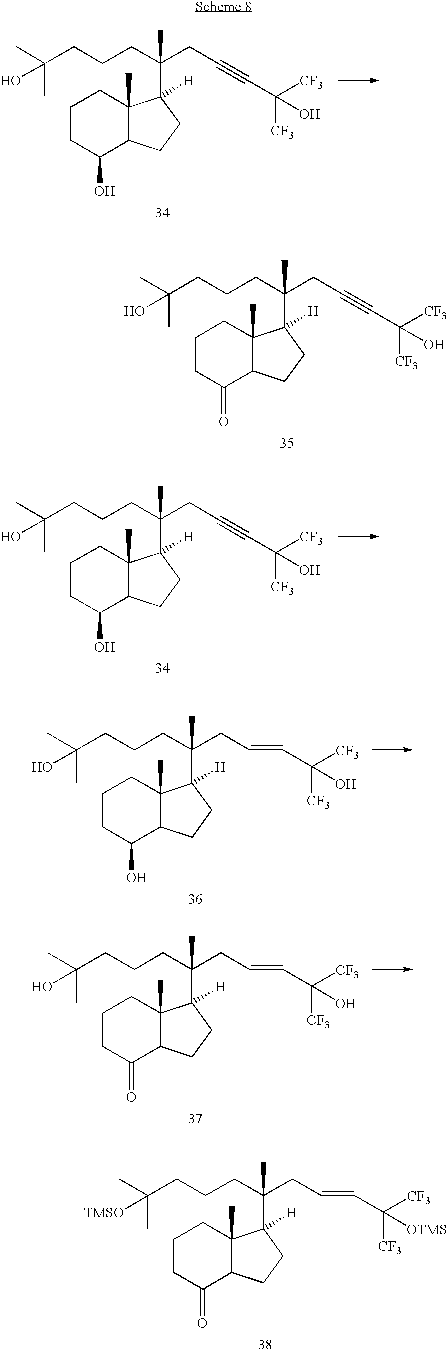

Scheme 9 shows the coupling of ketones 35 and 38 with phosphine oxide 27 to provide compounds 4 and 5.

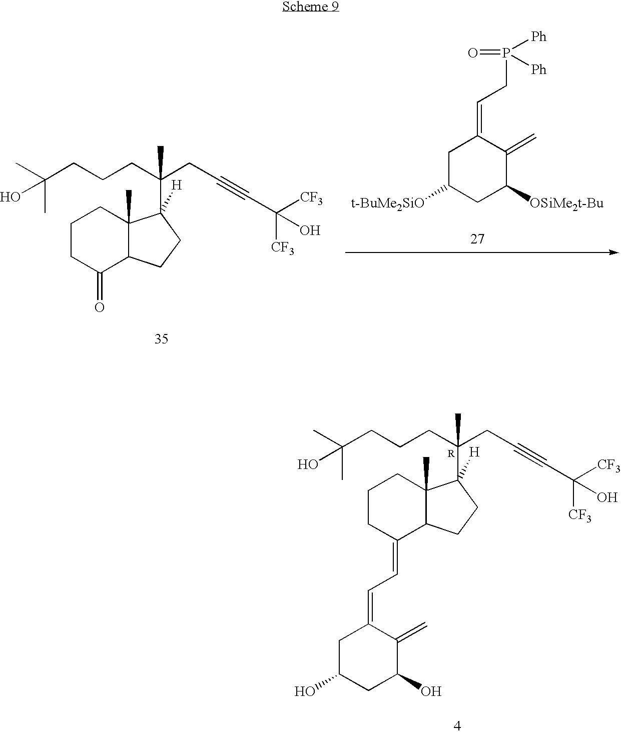

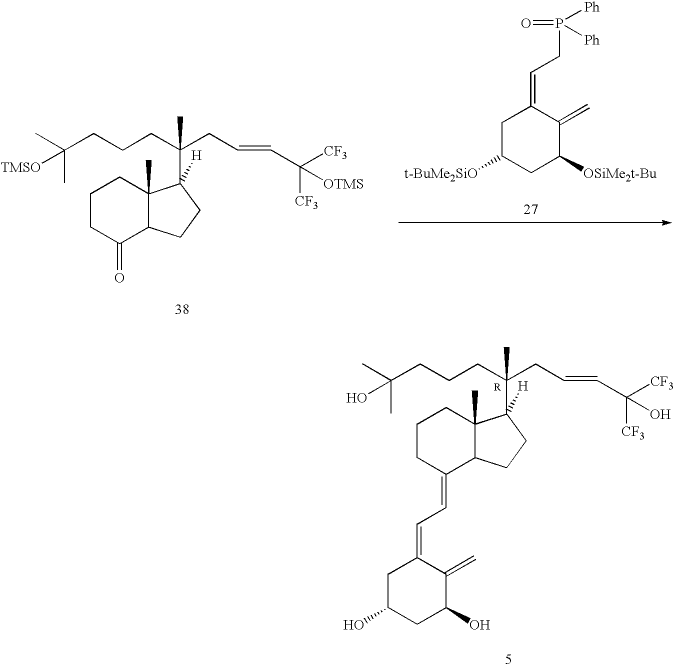

Chiral syntheses can result in products of high stercoisomer purity. However, in some cases, the stereoisomer purity of the product is not sufficiently high. The skilled artisan will appreciate that the separation methods described herein can be used to further enhance the stercoisomer purity of the vitamin D₃-epimer obtained by chiral synthesis.

Any novel syntheses, described herein, of the compounds of the invention, and of intermediates thereof, are also intended to be included within the scope of the present invention.

### EXEMPLIFICATION OF THE INVENTION

The invention is further illustrated by the following examples which should in no way should be construed as being further limiting.

### Synthesis of Compounds of the Invention

### Experimental

All operations involving vitamin D₃ analogs were conducted in amber-colored glassware in a nitrogen atmosphere. Tetrahydrofuran was distilled from sodium-benzophenone ketyl just prior to its use and solutions of solutes were dried with sodium sulfate. Melting points were determined on a Thomas-Hoover capillary apparatus and are uncorrected. Optical rotations were measured at 25° C. ¹H NMR spectra were recorded at 400 MHz in CDCl₃ unless indicated otherwise. TLC was carried out on silica gel plates (Merck PF-254) with visualization under short-wavelength UV light or by spraying the plates with 100% phosphomolybdic acid in methanol followed by heating. Flash chromatography was carried out on 40-65 μm mesh silica gel. Preparative HPLC was performed on a 5×50 cm column and 15-30 μm mesh silica gel at a flow rate of 100 mL/min.

### Example 1

### Synthesis of (20S)-1,25-Dihydroxy-20-(5,5,5-trifluoro-4-hydroxy-4-trifluoromethyl-pent-2-ynyl)cholecalciferol (1)

### (1R,3aR,4S,7aR)-4-(tert-Butyl-dimethyl-silanyloxy)-1-[3-(tert-butyl-dimethyl-silanyloxy)-1-methylene-propyl]-7a-methyl-octahydro-indene (8)

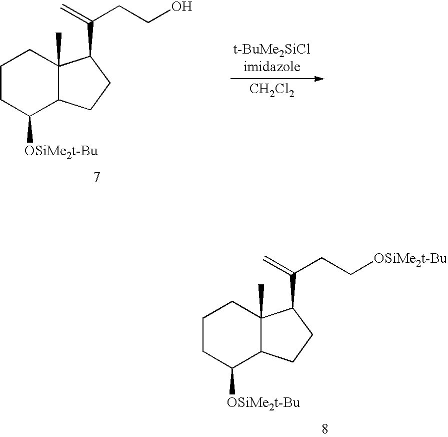

A 250 ml round bottom flask equipped with stir bar, Claisen adapter with rubber septum and nitrogen sweep was charged with 17.53 g (51.77 mmol) of 3-[(1R,3aR,4S, 7aR)-4-(tert-butyl-dimethyl-silanyloxy)-7a-methyl-octahydro-inden-1-yl]-but-3-en-1-ol and 75 ml of dichloromethane. A 7.05 g (103.54 mmol) imidazole was added followed by 9.36 g (62.124 mmol) of t-butyldimethylsilyl chloride. The mixture was stirred for 2.5 h.

The mixture was then diluted with 100 ml of water and extracted four times with 50 ml of dichloromethane. The combined organic layers were dried over Na₂SO₄ and evaporated.

The oil residue was chromatographed on column (400 cm³) using hexane, hexane:ethyl acetate (50:1, 25:1) as mobile phase and collecting ca. 40 ml fractions to give 22.32 g (95%) of product as colorless oil.

¹H NMR (CDCl₃): 4.87 (1H, s), 4.80 (1H, s), 4.02 (1H, br s), 3.67 (2H, t, J=7.3 Hz), 2.34-2.14 (2H, m), 2.06-2.00 (1H, m), 1.85-1.27 (9H, m), 1.20-1.08 (2H, m), 0.89 (18H, s), 0.79 (3H, s), 0.05 (6H, s), 0.02 (3H, s), 0.01 (3H, s)

### 2-[2-(tert-Butyl-dimethyl-silanyloxy)-ethyl]-2-[(1S,3aR,7aR)-4-(tert-butyl-dimethyl-silanyloxy)-71-methyl-octahydro-inden-1-yl]-cyclopropanecarboxylic acid ethyl ester (9)

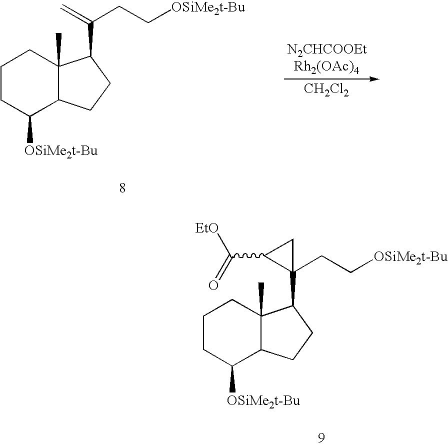

A 250 ml round bottom flask equipped with stir bar and Claisen adapter with rubber septum vas charged with 10.00 g (22.08 mmol) of (1R,3aR,4S,7aR)-4-(tert-butyl-dimethyl-silanyloxy)-1-[3-(tert-butyl-dimethyl-silanyloxy)-1-methylene-propyl]-7a-methyl-octahydro-indene, 200 mg of Rh₂(OAc)₄ and 40 ml of dichloromethane. A solution of 5.304 g (46.486 mmol) of ethyl diazoacetate in 30 ml of dichloromethane was added dropwise (12 ml/h) at room temperature.

The reaction mixture was concentrated in vacuo and the remaining residue was filtrated on column (200 cm³) using hexane:ethyl acetate (1:1) as mobile phase. The solvent was evaporated and the oil residue was chromatographed on column (250 cm³) using hexane:ethyl acetate (25:1, 10:1 and 5:1) as mobile phase to give 8.44 g (71%) of products as a mixture of isomers.

### {2-[2-(tert-Butyl-dimethyl-silanyloxy)-ethyl]-2-[(1S,3aR,4S,7aR)-4-(tert-butyl-dimethyl-silanyloxy)-7a-methyl-octahydro-inden-1-yl]-cyclopropyl}-methanol (10)

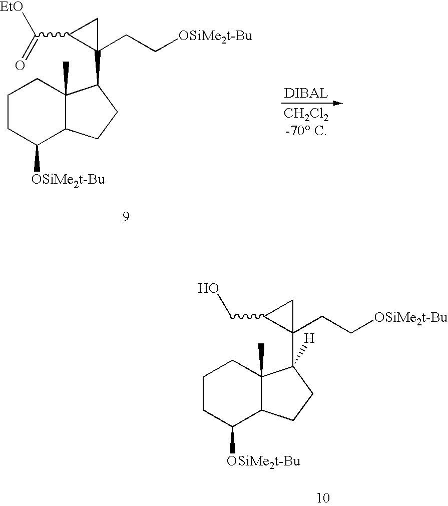

A 50 ml round bottom flask equipped with stir bar and Claisen adapter with rubber septum was charged with 4.140 g (7.682 mmol) of 2-[2-(tert-butyl-dimethyl-silanyloxy)-ethyl]-2-[(1S,3aR,4S,7aR)-4-(tert-butyl-dimethyl-silanyloxy)-7a-methyl-octahydro-inden-1-yl]-cyclopropanecarboxylic acid ethyl ester and 20 ml of dichloromethane. The reaction mixture was cooled to −70° C. and 10.0 ml (15.0 mmol) of 15M DIBAL-H in toluene was added dropwise during 45 min. The reaction was stirred at −70° C. for 1 h and then 5 ml of saturated solution of ammonium chloride was added dropwise.

The mixture was dissolved by the addition of 100 ml of water and 50 ml of 1N HCl, extracted three times with 50 ml of ethyl acetate, dried over Na₂SO₄ and evaporated.

The oil residue was chromatographed on column (200 cm³) using hexane:ethyl:acetate (10:1, 3:1) as mobile phase. The fractions containing product were pooled and evaporated to give 3.610 g, (940%) of products (mixture of isomers) as colorless oil.

### 2-[2-(tert-Butyl-dimethyl-silanyloxy)-ethyl]-2-[(1S,3aR,4S,7aR)-4-(tert-butyl-dimethyl-silanyloxy)-7a-methyl-octahydro-inden-1-yl]-cyclopropanecarbaldehyde (11)

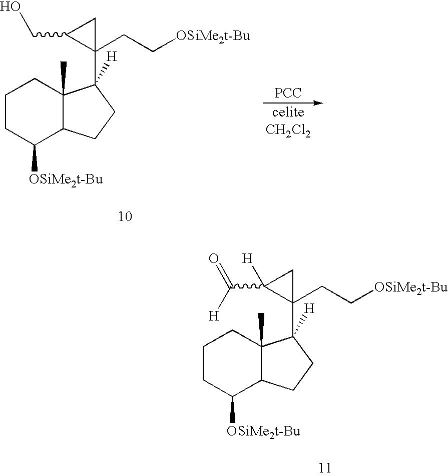

A 250 ml round bottom flask equipped with stir bar and Claisen adapter with rubber septum was charged with 6.074 g (28.178 mmol) of pyridinium chlorochromate, 7.00 g of celite and 100 ml of dichloromethane. A 6.970 g (14.027 mmol) of {2-[2-(tert-butyl-dimethyl-silanyloxy)-ethyl]-2-[(1S,3aR,4S,7aR)-4-(tert-butyl-dimethyl-silanyloxy)-7a-methyl-octahydro-inden-1-yl]-cyclopropyl}-methanol in 10 ml of dichloromethane was added dropwise and mixture was stirred in room temperature for 1 h.

The reaction mixture was filtrated through column with silica gel (200 cm³) and celite (2 cm) and using dichloromethane as a mobile phase. The fractions containing product were pooled and evaporated to give oil (ca. 5.71 g). Product was used to the next reaction without purification.

### 3-{2-[2-(tert-Butyl-dimethyl-silanyloxy)-ethyl]-2-[(1S,3aR,4S,7aR)-4-(tert-butyl-dimethyl-silanyloxy)-7a-methyl-octahydro-inden-1-yl]-cyclopropyl}-acrylic acid ethyl ester (12)

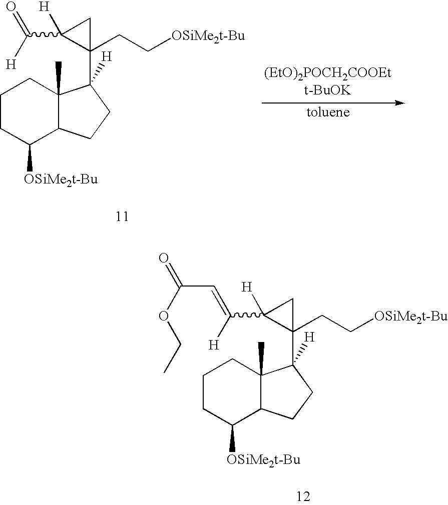

A 250 ml round bottom flask equipped with stir bar and Claisen adapter with rubber septum was charged with 80 ml of toluene and 35.0 ml (35.0 mmol) of 1M potassium tert-butoxide in tetrahydrofurane was added. A 7.850 g (35.015 mmol) of triethyl phosphonoacetate in 5 ml of toluene was added dropwise at ca. 5° C. The mixture was stirred at room temperature for 1 h. Then the mixture was cooled to −15° C. and crude (ca. 11.54 mmol) 2-[2-(tert-butyl-dimethyl-silanyloxy)-ethyl]-2-[(1S,3aR,4S, 7aR)-4-(tert-butyl-dimethyl-silanyloxy)-7a-methyl-octahydro-inden-1-yl]-cyclopropanecarbaldehyde in 5 ml of toluene was added and stirring was continued at −10° C. for 3 h.

The reaction mixture was quenched with 10 ml of aqueous saturated solution of ammonium chloride, diluted with 100 ml of saturated solution of ammonium chloride and extracted four times with 50 ml of toluene and then 50 ml of ethyl acetate. The organic layer was washed with 50 ml of brine, dried and evaporated.

The residue was purified over silica gel (200 cm³) using hexane:ethyl acetate (20:1) as a mobile phase to give 5.750 g (88%) of products (mixture of isomers).

### 7-(tert-Butyl-dimethyl-silanyloxy)-5-[(1R,3aR,4S,7aR)-4-(tert-butyl-dimethyl-silanyloxy)-7a-methyl-octahydro-inden-1-yl]-5-methyl-heptanoic acid ethyl ester (13)

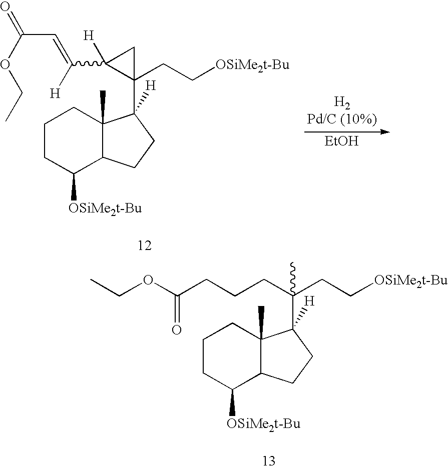

A 5.750 g (10.177 mmol) of 3-{2-[2-(tert-butyl-dimethyl-silanyloxy)-ethyl]-2-[(1S,3aR,4S,7aR)-4-(tert-butyl-dimethyl-silanyloxy)-7a-methyl-octahydro-inden-1-yl]-cyclopropyl}-acrylic acid ethyl ester was hydrogenated over 1.60 g of 10% Pd/C in 40 ml of ethanol at room temperature and atmospheric pressure of hydrogen. The reaction was monitoring by TLC (hexane:ethyl acetate-50:1).

After 18 h the catalyst was filtered off and solvent evaporated. The residue was purified over silica gel (300 cm³) using hexane:ethyl acetate (100:1, 50:1, 20:1) as a mobile phase to give 5.150 g (89%) of products (mixture of isomers).

### 8-(tert-Butyl-dimethyl-silanyloxy)-6-[(1R,3aR,4S,7aR)-4-(tert-butyl-dimethyl-silanyloxy)-7a-methyl-octahydro-inden-1-yl]-2,6-dimethyl-octan-2-ol (14)

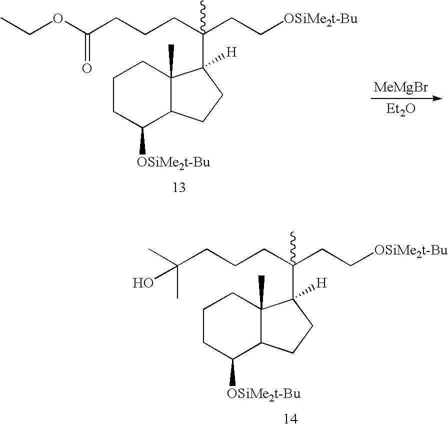

A 250 ml round bottom flask equipped with stir bar, Claisen adapter with rubber septum was charged with 5.110 g (8.980 mmol) of 7-(tert-butyl-dimethyl-silanyloxy)-5-[(1R,3aR,4S,7aR)-4-(tert-butyl-dimethyl-silanyloxy)-7a-methyl-octahydro-inden 1-yl]-5-methyl-heptanoic acid ethyl ester and 80 ml of diethyl ether. The solution was cooled in ace-water bath and 17.4 ml (54.3 mmol) of 3.12M solution of methylmagnesium bromide in diethyl ether was added dropwise. After completion of the addition the mixture was stirred at room temperature for 2.5 h then cooled again in an ice bath. A 10 ml of saturated solution of ammonium chloride was added dropwise. The resulting precipitate was dissolved by the addition of 50 ml of saturated solution of ammonium chloride. The aqueous layer was extracted three times with 100 ml of ethyl acetate. The combined organic layers were dried (Na₂SO₄) and evaporated. The product was used to the next reaction without farther purification.

### 3-[(1R,3aR,4S,7aR)-4-(tert-Butyl-dimethyl-silanyloxy)-7a-methyl-octahydro-inden-1-yl]-3,7-dimethyl-octane-1,7-diol (15 and 16)

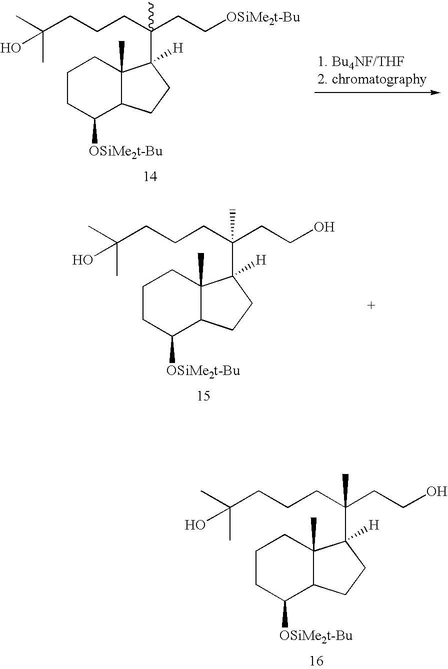

A 50 ml round bottom flask equipped with stir bar and Claisen adapter with rubber septum was charged with crude (ca. 8.98 mmol) 8-(tert-butyl-dimethyl-silanyloxy)-6-[4-(tert-butyl-dimethyl-silanyloxy)-7a-methyl-octahydro-inden-1-yl]-2,6-dimethyl-octan-2-ol, 10 ml of tetrahydrofurane and 15.0 ml 15.0 mmol) of 1M tetrabutylammonium fluoride in tetrahydrofurane. The reaction mixture was stirred at room temperature for 2.5 h.

The mixture was dissolved by the addition of 150 ml of ethyl acetate and extracted six times with 50 ml of water:brine (1:1) and 50 ml of brine, dried over Na₂SO₄ and evaporated.

The oil residue was chromatographed four times on columns (400 cm³) using hexane:ethyl acetate (1:1) as a mobile phase to give: 1^{st} 0.456 g (low polar epimer); 2^{nd}-0.852 g, (mixture of epimers); 3^{rd}-1.132 g (more polar epimer); All products 3.440 g (88% two steps):

### Low Polar Epimer: (3S)-3-[(1R,3aR,4S,7aR)-4-(tert-Butyl-dimethyl-silanyloxy)-7a-methyl-octahydro-inden-1-yl]-3,7-dimethyl-octane-1,7-diol

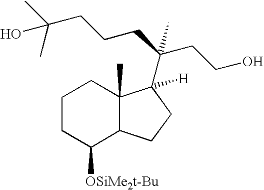

[α]_{D}³¹=+26.1° c=0.44, CHCl₃

¹H NMR(CDCl₃): 3.90 (1H, br s), 3.67 (2H, br t, J=8.1 Hz), 2.06-1.99 (1H, m), 1.87-1.50 (4H, m), 1.73 (2H, t, J=7.9 Hz), 1.40-1.06 (14H, m), 1.22 (6H, s), 1.06 (3H, s), 0.95 (3H, s), 1.96-0.82 (1H, m), 0.88 (9H, s), 0.00 (3H, s), −0.01 (3H, s)

¹³C NMR (CDCl₃): 71.03, 69.58, 59.79, 57.32, 52.99, 44.78, 43.81, 41.64, 41.58, 40.26, 38.68, 34.37, 29.48, 29.36, 25.86, 23.49, 22.78, 21.72, 18.18, 18.09, 17.78, 16.78, −4.70, −5.07

| | | | | |
|---|---|---|---|---|
| MS HRES | Calculated for: | C₂₆H₅₂O₃Si | [M + Na]⁺ | 463.3578 |
| | Observed: | | [M + Na]⁺ | 463.3580 |

### More Polar Epimer: (3R)-3-[(1R,3aR,4S,7aR)-4-(tert-butyl-dimethyl)-silanyloxy)-7a-methyl-octahydro-inden-1-yl]-3,7-dimethyl-octane-1,7-diol

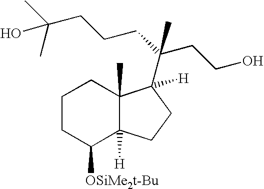

[α]_{D}³¹+22.7° c−0.44, CHCl₃

¹H NMR (CDCl₃): 3.99-3.97 (1H, m), 3.65-3.61 (2H, m), 1.97 (1H, br d, J=12.3Hz), 1.84-1.72 (1H, m), 1.66-1.50 (6H, m), 1.45-1.15 (14H, m), 1.21 (6H, s), 1.05 (3H, s), 0.95 (3H, s), 0.87 (9H, s), −0.01 (3H, s), −0.02 (3H, s)

¹³C NMR (CDCl₃): 71.05, 69.57, 59.47, 57.46, 53.02, 44.87, 43.90, 41.83, 41.61, 39.99, 38.93, 34.37, 29.43, 29.42, 25.87, 23.42, 22.84, 22.12, 18.57, 18.09, 17.81, 16.79, −4.69, −5.06

| | | | | |
|---|---|---|---|---|
| MS HRES | Calculated for: | C₂₆H₅₂O₃Si | [M + Na]⁺ | 463.3578 |
| | Observed: | | [M + Na]⁺ | 463.3575 |

### (3S)-3-[1R,3aR,4S,7aR)-4-(tert-Butyl-dimethyl-silanyloxy)-7a-methyl-octahydro-inden-1-yl]-7-hydroxy-3,7-dimethyl-octanal (17)

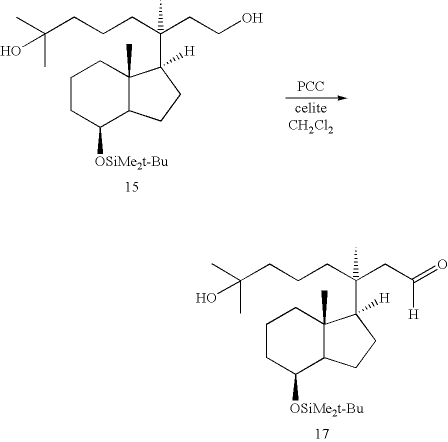

A 25 ml round bottom flask equipped with stir bar and Claisen adapter with rubber septum was charged with 1.572 g (7.292 mmol) of pyridinium chlorochromate, 1.60 g of celite and 25 ml of dichloromethane. A 1.607 g (3.646 mmol) of (3S)-3-[(1R, 3aR,4S,7aR)-4-(tert-butyl-dimethyl-silanyloxy)-7a-methyl-octahydro-inden-1-yl]-3,7-dimethyl-octane-1,7-diol in 6 ml of dichloromethane was added dropwise and mixture was stirred at room temperature for 1 h 45 min and additional portion 300 mg (1.392 mmol) of pyridinium chlorochromate was added. The reaction was stirred for next 1 h 15 min.

The reaction mixture was filtrated through column with silica gel (50 cm³) and celite (1 cm) using dichloromethane, dichloromethane:ethyl acetate (4:1). The fractions containing product were pooled and evaporated to give 1.58 g of product as yellow oil. The product was used to the next reaction without farther purification.

### (6S)-6-[(11R,3aR,4S,7aR)-4-(tert-Butyl-dimethyl-silanyloxy)-7a-methyl-octahydro-inden-1-yl]-2,6-dimethyl-non-8-yn-2-ol (18)

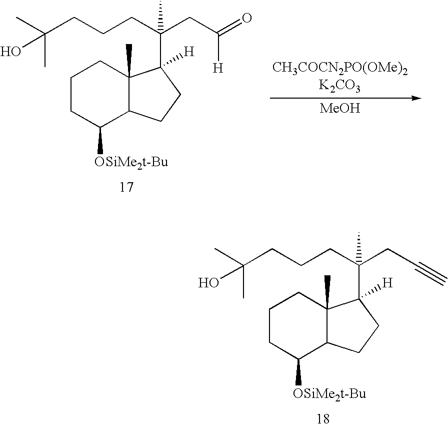

A 50 ml round bottom flask equipped with stir bar and Claisen adapter with rubber septum was charged with 1.58 g (3.601 mmol) of (3S)-3-[(1R,3aR,4S,7aR)-4-(tert-butyl-dimethyl-silanyloxy)-7a-methyl-octahydro-inden-1-yl]-7-hydroxy-3,7-dimethyl-octanal and 30 ml of methanol. A 1.416 g (7.37 mmol) of 1-diazo-2-oxo-propyl)-phosphonic acid dimethyl ester in 3 ml of methanol was added and the resulting mixture was cooled in an ice bath. A 1.416 g (10.245 mmol) of potassium carbonate was added and the reaction mixture was stirred in the ice bath for 30 min and then at room temperature for 3 h.

A 100 ml of water was added and the mixture was extracted three times with 80 ml of ethyl acetate, dried over Na₂SO₄ and evaporated.

The oil residue was chromatographed on column (250 cm³) using hexane:ethyl:acetate (7:1) as mobile phase. Fractions containing product were pooled and evaporated to give 1.310 g (83%, 2 steps) of product as colorless oil.

[α]_{D}³⁰=+15.7° c=0.61. CHCl₃

¹H NMR (CDCl₃): 3.98 (1H, br s), 2.28 (2H, d, J=2.1 Hz), 1.95-1.91 (2H, m), 1.78 (1H, dt, J=13.4, 3.8 Hz), 1.68-1.62 (1H, m), 1.58-1.48 (6H, m), 1.44-1.17 (15H, m), 1.22 (6H, s), 1.04 (3H, s), 1.00 (3H, s), 0.93-0.83 (1H, m), 0.88 (9H, s), −0.00 (3H, s), −0.01 (3H, s)

¹³C NMR (CDCl₃): 83.09, 71.03, 69.84, 69.64, 56.68, 52.95, 44.80, 43.71, 41.31, 40.21, 39.28, 34.33, 29.44, 29.29, 28.80, 25.85, 22.74, 22.69, 22.18, 18.14, 18.05, 17.73, 16.68, −4.77, −5.13

| | | | | |
|---|---|---|---|---|
| MS HRES | Calculated for: | C₂₇H₅₀O₂Si | [M + Na]⁺ | 457.3472 |
| | Observed: | | [M + Na]⁺ | 457.3473 |

### (1R,3aR,4S,7aR)-4-(tert-Butyl-dimethyl-silanyloxy)-1-[(1S)-1,5-dimethyl-1-prop-2-ynyl-5-trimethylsilanyloxy-hexyl]-7a-methyl-octahydro-indene (19)

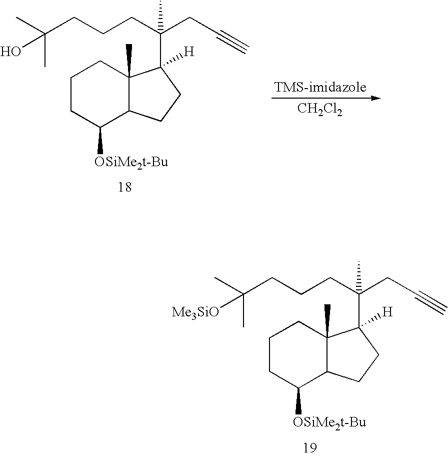

A 50 ml round bottom flask equipped with stir bar and Claisen adapter with rubber septum was charged with 1.300 g (2.990 mmol) of (6S)-6-[(1R,3aR,4S,7aR)-4-(tert-butyl-dimethyl-silanyloxy)-7a-methyl-octahydro-inden-1-yl]-2,6-dimethyl-non-8-yn-2-ol and 25 ml of dichloromethane. A 2900 ml (13.63 mmol) of 1-(trimethylsilyl)imidasole was added dropwise. The mixture was stirred at room temperature for 1 h.

A 100 ml of water was added and the mixture was extracted three times with 80 ml of hexane, dried over Na₂SO₄ and evaporated.

The oil residue was chromatographed on column (75 cm³) using hexane:ethyl acetate (25:1) as mobile phase. Fractions containing product were pooled and evaporated to give 1.409 g (93%) of product as colorless oil.

¹H NMR (CDCl₃): 3.98 (1H, br s), 2.27 (2H, d, J=2.9 Hz), 1.97-1.91 (2H, m), 1.82-1.75 (1H, m), 1.69-1.62 (1H, m), 1.59-1.50 (2H, m), 1.42-1.20 (12H, m), 1.20 (6H, s), 1.05 (3H, s), 1.00 (3H, s), 0.93-0.85 (1H, m), 0.88 (9H, s), 0.10 (9H, s), 0.00 (3H, s), −0.01 (3H, s)

### (6S)-6-[(1R,3aR,4S,7aR)-4-(tert-Butyl-dimethyl-silanyloxy)-7a-methyl-octahydro-inden-1-yl]-1,1,1-trifluoro-6,10-dimethyl-2-trifluoromethyl-10-trimethylsilanyloxy-undec-3-yn-2-ol (20)

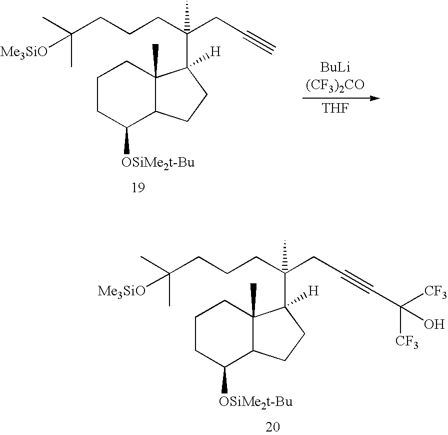

A two neck 50 ml round bottom flask equipped with stir bar, Claisen adapter with rubber septum and funnel (with cooling bath) was charged with 1.390 g (2.742 mmol) of (1R,3aR,4S,7aR)-4-(tert-butyl-dimethyl-silanyloxy)-1[-(1S)-1,5-dimethyl-1-prop-2-ynyl-5-trimethylsilanyloxy-hexyl]-7a-methyl-octahydro-indene and 30 ml of tetrahydrofurane. The funnel was connected to container with hexafluoroacetone and cooled (acetone, dry ice). The reaction mixture was cooled to −70° C. and 5.00 ml (8.00 mmol) of 1.6M n-butyllithium in tetrahydrofurane was added dropwise. After 30 min hexafluoroacetone was added (the contener's valve was opened three times). The reaction was stirred at −70° C. for 2 h then 5.0 ml of saturated solution of ammonium chloride was added.

The mixture was dissolved by the addition of 100 ml of saturated solution of ammonium chloride and extracted three times with 80 ml of ethyl acetate, dried over Na₂SO₄ and evaporated.

The oil residue was chromatographed twice to remove a large amount of polymer compounds. The first column (100 cm³) using hexane:ethyl acetate (10:1) as mobile phase. The second column (100 cm³) using hexane:ethyl acetate (25:1, 15.1) as mobile phase. Fractions containing product were pooled and evaporated to give 1.959 g of colorless oil. Product was used to the next reaction without farther purification.

### (6S)-1,1,1-Trifluoro-6-[(1R,3aR,4S,7aR)-4-hydroxy-7a-methyl-octahydro-inden-1-yl]-6,10-dimethyl-2-trifluoromethyl-undec-3-yne-2,10-diol (21)

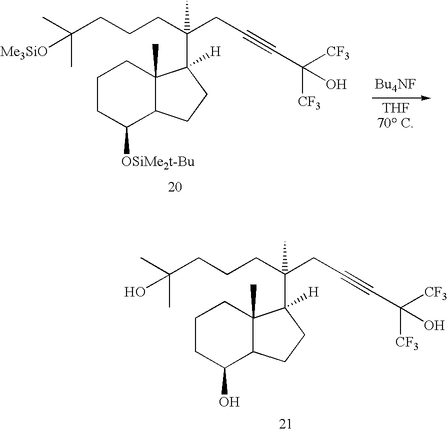

A 25 ml round bottom flask equipped with stir bar and Claisen adapter with rubber septum was charged with crude (ca. 2.74 mmol) (6S)-6-[(1R,3aR,4S,7aR)-4-(tert-butyl-dimethyl-silanyloxy)-7a-methyl-octahydro-inden-1-yl]-1,1,1-trifluoro-6,10-dimethyl-2-trifluoromethyl-10-trimethylsilanyloxy-1-inden-3-yn-2-ol and 12.0 ml (12.0 mmol) of 1M tetrabutylammonium fluoride in tetrahydrofurane and reaction was stirred at 70° C. After 18 h new portion 5.0 ml of 1M tetrabutylammonium fluoride in tetrahydrofurane was added. The reaction mixture was stirred at 70° C. for next 80 h.

The mixture was dissolved by the addition of 150 ml of ethyl acetate and extracted six times with 50 ml of water:brine (1:1) and 50 ml of brine and dried over Na₂SO₄ and evaporated.

The oil residue was chromatographed on column (200 cm³) using hexane:ethyl:acetate (3:1, 2:1) as mobile phase. The fractions containing product were pooled and evaporated. The residue was crystallized from hexane-ethyl acetate to give 917 mg (69%, two steps) of product as a white crystal.

m.p. 146-147° C.

[α]_{D}³⁰=−3.5° c=0.43, CHCl₃

¹H NMR (CDCl₃): 4.08 (1H, br s), 2.45 (1H, AB, J=17 Hz), 2.36 (1H, AB, J=17 Hz), 1.98-1.92 (1H, m), 1.85-1.74 (2H, m), 1.67-1.18 (18H, m), 1.25 (6H, s), 1.07 (3H, s), 1.02 (3H, s)

| | | | | |
|---|---|---|---|---|
| MS HRES | Calculated for: | C₂₄H₃₆F₆O₃ | [M + Na]⁺ | 509.2461 |
| | Observed: | | [M + Na]⁺ | 509.2459 |

### (1R,3aR,4S,7aR)-7a-Methyl-1-[(1S)-6,6,6-trifluoro-5-hydroxy-1-(4-hydroxy-4-methyl-pentyl)-1-methyl-5-trifluoromethyl-hex-3-ynyl]-octahydro-inden-4-one (22)

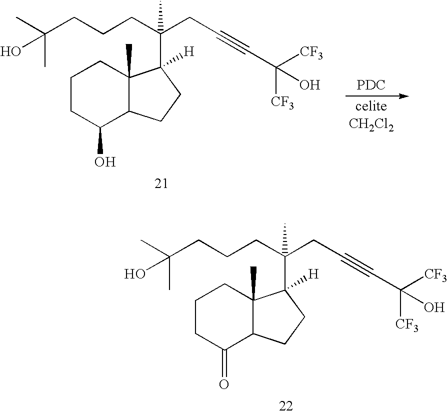

A 25 ml round bottom flask equipped with stir bar and Claisen adapter with rubber septum was charged with 300 mg (0.617 mmol) of (6S)-1,1,1-trifluoro-6-[(1R, 3aR,4S,7aR)-4-hydroxy-7a-methyl-octahydro-inden-1-yl]-6,10-dimethyl-2-trifluoromethyl-undec-3-yne-2,10-diol and 10 ml of dichloromethane. A 696 mg (1.851 mmol) of pyridinium dichromate and 710 mg of celite were added and mixture was stirred in room temperature for 3 h.

The reaction mixture was filtrated through column with silica gel (50 cm³) and celite (2 cm) and using dichloromethane:ethyl acetate (4:1) as a mobile phase. The fractions containing product were pooled and evaporated to give yellow oil. The product was used to the next reaction without farther purification.

### (20S)-1,25-Dihydroxy-20-(5,5,5-trifluoro-4-hydroxy-4-trifluoromethyl-pent-2-ynyl)cholecalciferol (1)

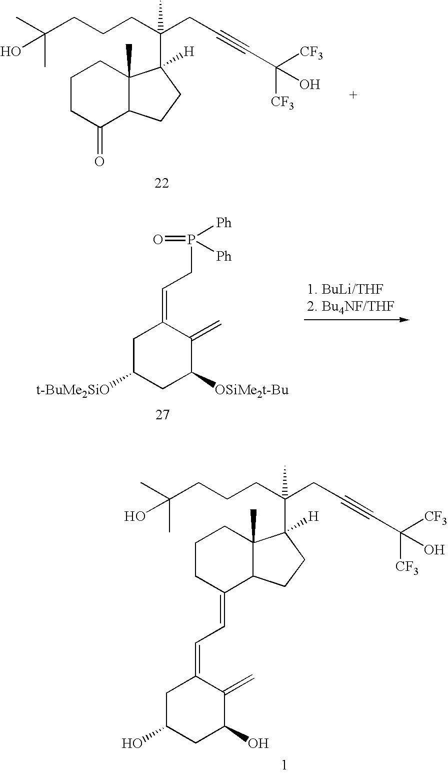

A 25 ml round bottom flask equipped with stir bar and Claisen adapter with rubber septum was charged with 1.798 g (3.084 mmol) of (1S,5R)-1,5-bis-((tert-butyldimethyl)silanyloxy)-3-[2-(diphenylfosphinoyl)-eth-(Z)-ylidene]-2-methylene-cyclohexane and 1 ml of tetrahydrofurane. The reaction mixture was cooled to −78° C. and 1.9 ml (3.04 mmol) of 1.6M n-butyllithium in tetrahydrofurane was added dropwise. The resulting deep red solution was stirred at −78° C. for 20 min and crude (ca 0.617 mmol)(1R,3aR,4S,7aR)-7a-methyl-1-[(1S)-6,6,6-trifluoro-5-hydroxy-1-(4-hydroxy-4-methyl-pentyl)-1-methyl-5-trifluoromethyl-hex-3-ynyl]-octahydro-inden-4-one was added dropwise in 1.5 ml of tetrahydrofurane. The reaction mixture was stirred for 5 h and then the bath was removed and the mixture was poured into 50 ml of ethyl acetate and 100 ml of brine. The water fraction was extracted three times with 50 ml of ethyl acetate, dried over Na₂SO₄ and evaporated.

The oil residue was chromatographed on column (75 cm³, protected from light) using hexane:ethyl acetate (5:1) as mobile phase. Fractions containing product were pooled and evaporated to give colorless oil (293 mg) which was treated with 5 ml of 1M tetrabutylammonium fluoride in tetrahydrofurane. The reaction mixture was stirred at room temperature for 40 h.

The mixture was dissolved by the addition of 150 ml of ethyl acetate and extracted six times with 50 ml of water-brine (1:1) and 50 ml of brine, dried over Na₂SO₄ and evaporated.

The oil residue was chromatographed on column (50 cm³, protected from light) using ethyl acetate as mobile phase Fractions containing product were pooled and evaporated to give product as colorless oil. Oil was dissolved in methyl acetate and evaporated (4 times) to give 190 mg (50% three steps) of product as white foam.

[α]_{D}³⁰=−4.6° c=0.3, CHCl₃

UV λmax (EtOH): 205.50 nm (ε 16586), 266.00 nm (ε 4319)

¹H NMR (CDCl₃): 6.36 (1H, d, J=11.3 Hz), 6.23 (1H, br s), 6.00 (1H, d, J=11.1 Hz), 5.32 (1H, s), 4.98 (1H, s), 4.43 (1H, dd, J=7.7, 4.3 Hz), 4.25-4.20 (1H, m), 2.82-2.79 (1H, m), 2.59 (1H, dd, J=13.1, 3.1 Hz), 2.44 (1H, AB, J=17.2 Hz), 2.37 (1H, AB, J=17.2 Hz), 2.30 (1H, dd, J=13.2, 6.2 Hz,), 2.06-1.87 (4H, m), 1.72-1.36 (11H, m), 1.26-1.21 (1H, m), 1.24 (6H, s), 0.99 (3H, s), 0.64 (3H, s)

¹³C NMR (CDCl₃): 147.48, 142.29, 133.16, 124.72, 121.32 (q, J=142.7 Hz), 117.59, 11.68, 90.08, 72.62, 71.39, 70.73, 66.89, 57.28, 56.52, 46.65, 45.18, 43.20, 42.81, 41.04, 40.89, 40.03, 29.79, 29, 35, 28.95, 23.45, 22.86, 22.60, 21.84, 17.77, 14.93

| | | | | |
|---|---|---|---|---|
| MS HRES | Calculated for: | C₃₃H₄₆F₆O₄ | [M + Na]⁺ | 643.3192 |
| | Observed: | | [M + Na]⁺ | 643.3192 |

### Example 2

### Synthesis of (20S)-1α-Fluoro-25-hydroxy-20-(5,5,5-trifluoro-4-hydroxy-4-trifluoromethyl-pent-2-ynyl)-cholecalciferol (6)

### (1R,3aR,4S,7aR)-7a-Methyl-1-[(1S)-6,6,6-trifluoro-1-methyl-1-(4-methyl-4-trimethylsilanyloxy-pentyl)-5-trifluoromethyl-5-trimethylsilanyloxy-hex-3-ynyl]-octahydro-inden-4-one (28)

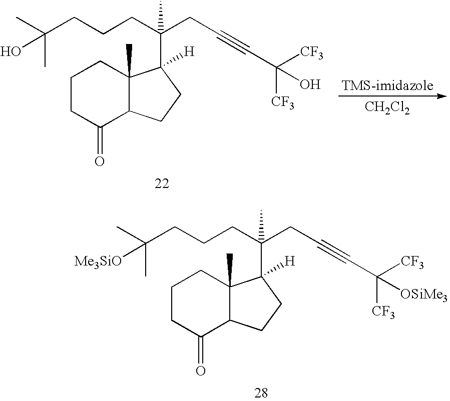

A 25 ml round bottom flask equipped with stir bar and Claisen adapter with rubber septum was charged with 585 mg (1.207 mmol) of (1R,3aR,4S,7aR)-7a-methyl-1-[(1S)-6,6,6-trifluoro-5-hydroxy-1-(4-hydroxy-4-methyl-pentyl)-1-methyl-5-trifluoromethyl-hex-3-ynyl]-octahydro-inden-4-one and 10 ml of dichloromethane. A 1.5 ml (10.2 mmol) of 1-(trimethylsilyl)imidazole was added dropwise. The mixture was stirred at room temperature for 3 h.

A 150 ml of ethyl acetate was added and the mixture was washed three times with 50 ml of water, died over Na₂SO₄ and evaporated.

The oil residue was chromatographed on column (50 cm³) using hexane:ethyl:acetate (10:1) as mobile phase. Fractions containing product were pooled and evaporated to give 660 mg (87%) of product as colorless oil.

¹H NMR (CDCl₃): 2.44-2.39 (3H, m), 2.32-2.16 (2H, m), 2.10-1.99 (2H, m), 1.95-1.84 (2H, m), 1.77-1.56 (4H, m), 1.38-1.19 (7H, m), 1.20 (6H, s), 1.03 (3H, s), 0.74 (3H, s), 0.28 (9H, s), 0.10 (9H, s)

### (20S)-1α-Fluoro-25-hydroxy-20-(5,5,5-trifluoro-4-hydroxy-4-trifluoromethyl-pent-2-ynyl)-cholecalciferol (6)

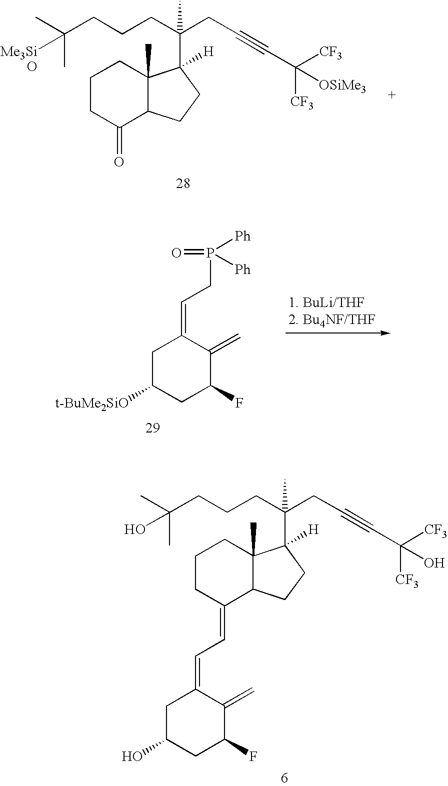

A 25 ml round bottom flask equipped with stir bar and Claisen adapter with rubber septum was charged with 495 mg (1.052 mmol) of (1S,5R)-1-((tert-butyldimethyl)silanyloxy)-3-[2-(diphenylfosphinoyl)-eth-(Z)-ylidene]-5-fluoro-2-methylene-cyclohexane and 10 ml of tetrahydrofurane. The reaction mixture was cooled to −70° C. and 0.65 ml (1.04 mmol) of 1.6M n-butyllithium was added dropwise. The resulting deep red solution was stirred at −70° C. for 20 min and 300 mg (0.477 mmol) of (1R,3aR,4S,7aR) 7a-methyl-1-[(1S)-6,6,6-trifluoro-1-methyl-1-(4-methyl-4-trimethylsilanyloxyl-pentyl)-5-trifluoromethyl-5-trimethylsilanyloxy-hex-3-ynyl]-octahydro-inden-4-one was added dropwise in 1.5 ml of tetrahydrofurane. The reaction mixture was stirred for 4 h and then the dry ice was removed from bath and the solution was allowed to warm up to −40° C. in 1 h. The mixture was poured into 50 ml of ethyl acetate and 100 ml of brine. The water fraction was extracted three times with 50 ml of ethyl acetate, dried over Na₂SO₄ and evaporated.

The oil residue was chromatographed on column (50 cm³, protected from light) using hexane:ethyl acetate (10:1) as mobile phase. Fractions containing product were pooled and evaporated to give colorless oil (ca. 429 mg) which was treated with 10 ml of 1M tetrabutylammonium fluoride in tetrahydrofurane. The reaction mixture was stirred at room temperature for 18 h.

The mixture was dissolved by the addition of 150 ml of ethyl acetate and extracted six times with 50 ml of water:brine (1:1) and 50 ml of brine, dried over Na₂SO₄ and evaporated.

The oil residue was chromatographed on column (50 cm³, protected from light) using ethyl acetate:hexane (1:1) as mobile phase. Fractions containing product were pooled and evaporated to give product as colorless oil. The product was dissolved in methyl acetate and evaporated (2 times) to give 274 mg 92%) of product as white foam.

[α]_{D}³⁰=+27.0 c=0.50, EtOH

UV λmax (EtOH): 212 nm (ε 34256), 243 nm (ε 15866), 271 nm (ε 16512)

| | | | | |
|---|---|---|---|---|
| MS HRES | Calculated for: | C₃₃H₄₅F₇O₃ | [M + Na]⁺ | 645.3149 |
| | Observed: | | [M + Na]⁺ | 645.3148 |

### Example 3

### Synthesis of (20S)-1,25-Dihydroxy-20-(5,5,5-trifluoro-4-hydroxy-4-trifluoromethyl-pent-(2Z)-enyl)cholecalciferol (2)

### (3Z,6S)-1,1,1-Trifluoro-6-[(1R,3aR,4S,7aR)-4-hydroxy-7a-methyl-octahydro-inden-1-yl]-6,10-dimethyl-2-trifluoromethyl-undec-3-ene-2,10-diol (23)

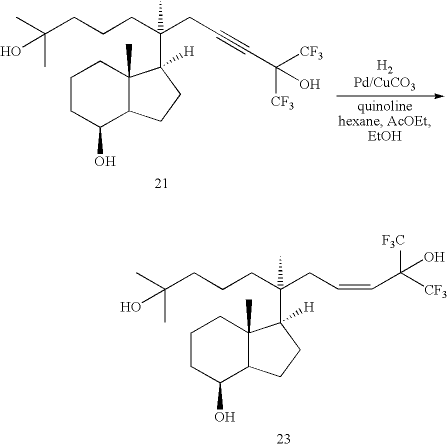

A 25 ml round bottom flask was charged with 250 mg (0.514 mmol) of (6S)-1,1,1-trifluoro-6-[(1R,3aR,4S,7aR)-4-hydroxy-7a-methyl-octahydro-inden-1-yl]-6,10-dimethyl-2-trifluoromethyl-undec-3-yne-2,10-diol, 70 mg of 5% Pd/CaCO₃, 6.0 ml of hexane, 2.4 ml of ethyl acetate and 0.23 ml of solution of quinoline in ethanol (prepared from 3.1 ml of ethanol and 168 μl of quinoline).

The substrate was hydrogenated at ambient temperature and atmospheric pressure of hydrogen. The reaction was monitoring by TLC (hexane:ethyl acetate—2:1). After 7 h the catalyst was filtered off and solvent evaporated. The residue was purified over silica gel (125 cm³) using hexane:ethyl acetate (2:1) as a mobile phase. Fractions containing product were pooled and evaporated to give 243 mg (97%) of product as colorless oil.

¹H NMR (CDCl₃): 6.14-6.05 (1H, m), 5.49 (1H, d, J=12.5 Hz), 4.08 (1H, br s), 2.83 (1H, dd, J=15.9, 9.7 Hz), 2.48-2.38 (1H, m), 1.85-1.75 (2H, m), 1.65-1.20 (17H, m), 1.22 (3H, s), 1.20 (3H, s), 1.08 (3H, s), 1.03-0.96 (1H, m), 1.00 (3H, s)

¹³C NMR (CDCl₃): 140.22, 117.44, 71.79, 69.66, 56.74, 52.58, 44.11, 43.45, 41.19, 40, 24, 39.64, 36.88, 33.44, 30.09, 28.88, 22.55, 22.21, 21.70, 17.63, 17.58, 16.54 (1R,3aR,4S,7aR)-7a-Methyl-1-[(1S,3Z)-6,6,6-trifluoro-5-hydroxy-1-(4-hydroxy-4-methyl-pentyl)-1 methyl-5-trifluoromethyl-hex-3-enyl]-octahydro-inden-4-one (24)

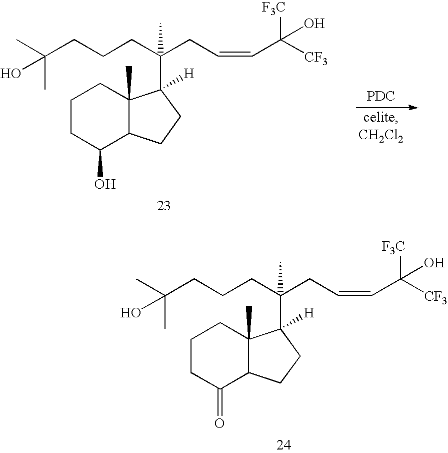

A 25 ml round bottom flask equipped with stir bar and Claisen adapter with rubber septum was charged with 290 mg (0.594 mmol) of (3Z,6S)-1,1,1-trifluoro-6-[(1R,3aR,4S,7aR)-4-hydroxy-7a-methyl-octahydro-inden-1-yl]-6,10-dimethyl-2-trifluoromethyl-undec-3-ene-2,10-diol and 10 ml of dichloromethane. A 700 mg (1.861 mmol) pyridinium dichromate and 750 mg of celite was added and mixture was stirred in room temperature for 3 h.

The reaction mixture was filtrated through column with silica gel (75 cm³) and celite (2 cm) and using dichloromethane:ethyl acetate (4:1) as a mobile phase. The fractions containing product were pooled and evaporated to give yellow oil. The product was used to the next reaction without farther purification.

### (20S)-1,25-Dihydroxy-20-(5,5,5-trifluoro-4-hydroxy-4-trifluoromethyl-pent-(2Z)-enyl)cholecalciferol (2)

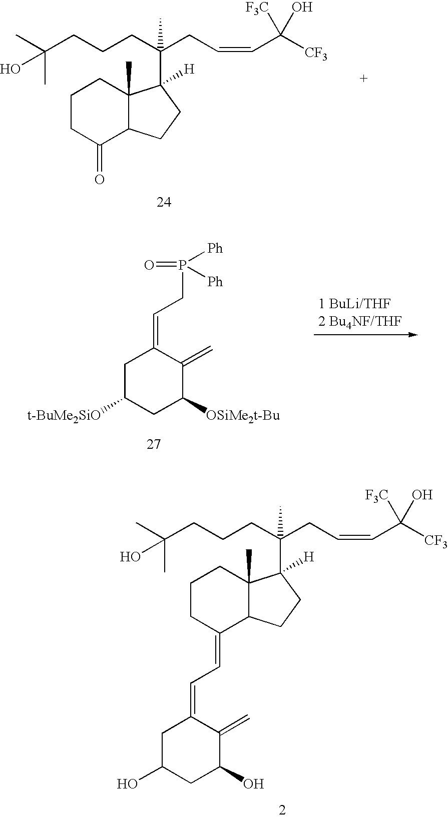

A 25 ml round bottom flask equipped with stir bar and Claisen adapter with rubber septum was charged with 1.800 g (3.088 mmol) of (1S,5R)-1,5-bis-((tert-butyldimethyl)silanyloxy)-3-[2-(diphenylfosphinoyl)-eth-(Z)-yliden]-2-methylene-cyclohexane and 10.0 ml of tetrahydrofurane. The reaction mixture was cooled to −78° C. and 1.9 ml (3.04 mmol) of 1.6M n-butyllithium in tetrahydrofurane was added dropwise. The resulting deep red solution was stirred at −78° C. for 20 min and 278 mg (0.571 mmol) of (1R,3a,4S,7aR)-7a-methyl-1-[(1S,3Z)-6,6,6-trifluoro-5-hydroxy-1-(4-hydroxy-4-methyl-pentyl)-1-methyl-5-trifluoromethyl-hex-3-enyl]-octahydro-inden-4-one was added dropwise in 1.5 ml of tetrahydrofurane. The reaction mixture was stirred for 5 h (last 0.5 h at −20° C.) and then the bath was removed and the mixture was poured into 50 ml of ethyl acetate and 100 ml of brine. The water fraction was extracted three times with 50 ml of ethyl acetate, dried over Na₂SO₄ and evaporated.

The oil residue was chromatographed on column (75 cm³, protected from light) using hexane:ethyl acetate (4:1) as mobile phase. Fractions containing product were pooled and evaporated to give colorless oil (309 mg) which was treated with 5 ml of 1M tetrabutylammonium fluoride in tetrahydrofurane. The reaction mixture was stirred at room temperature for 22 h.

The mixture was dissolved by the addition of 150 ml of ethyl acetate and extracted six times with 50 ml of water:brine (1:1) and 50 ml of brine, dried over Na₂SO₄ and evaporated.

The oil residue was chromatographed on column (50 cm³, protected from light) using ethyl acetate as mobile phase. Fractions containing product were pooled and evaporated to give product as colorless oil. Oil was dissolved in methyl acetate and evaporated (4 times) to give 192 mg (54%, two steps) of product as white foam.

UV λmax (EtOH) 204.08 nm (ε 27522), 266.03 nm (ε 20144)

¹H NMR (CDCl₃): 6.37 (1H, d, J=11.1 Hz), 6.10 (1H, ddd, J=12.5, 9.0, 6.0 Hz), 6.00 (1H, d, J=11.3 Hz), 5.47 (1H, d, J=12.2 Hz), 5.32 (1H, s), 5.07 (1H, br, s), 4.99 (1H, s), 4.43 (1H, dd, J=37.8, 4.2 Hz), 4.25-4.20 (1H, m), 2.85-2.79 (2H, m), 2.59 (1H, dd, J==13.4, 3.0 Hz), 2.46 (1H, dd, J=16.4, 4.9 Hz), 2.31 (1H, dd, J=13.4, 6.4 Hz), 2.04-1.97 (3H, m), 1.90 (1H, ddd, J=12.0, 8.2, 3.2 Hz), 1.76-1.20 (17H, m), 1.21 (3H, s), 1.20 (3H, s), 1.06-1.00 (1H, m), 0.96 (3H, s), 0.64 (3H, s)

¹³C NMR (CDCl₃): 147.51, 142.74, 140.17, 132.92, 124.88, 122.95 (q, J=142.6 Hz), 122.80 (q, J=141.9 Hz), 117.52, 117.39, 111.65, 71.94, 70.73, 66.88, 56.86, 56.65, 46.79, 45.20, 43.95, 42.83, 41.06, 40.09, 39.75, 37.22, 30.35, 29.05, 28.82, 23.58, 22.50, 22.19, 21, 93, 17.53, 15.04

| | | | | |
|---|---|---|---|---|
| MS HRES | Calculated for: | C₃₃H₄₈F₆O₄ | [M + Na]⁺ | 645.3349 |
| | Observed: | | [M + Na]⁺ | 645.3350 |

### Example 4

### Synthesis of (20S)-1,25-Dihydroxy-20-[(2E)-5,5,5-trifluoro-4-hydroxy-4-trifluoromethyl-pent-2-enyl]-cholecalciferol (3)

### (3E,6S)-1,1,1-Trifluoro-6-[(1R,3aR,4S,7aR)-4-hydroxy-7a-methyl-octahydro-inden-1-yl]-6,10-dimethyl-2-trifluoromethyl-undec-3-ene-2,10-diol (25)

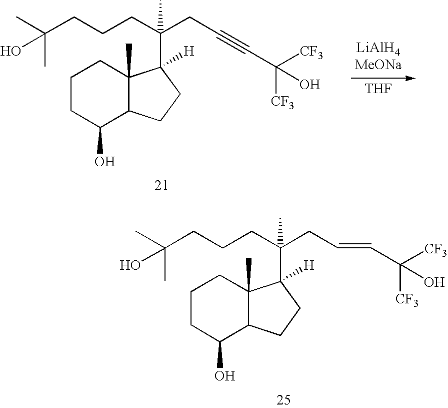

A 25 ml round bottom flask equipped with stir bar and condenser with nitrogen sweep was charged with 4.0 ml (4.0 mmol) of 1M lithium aluminum hydride in tetrahydrofurane. The mixture was cooled to 0° C. and 216 mg (4.00 mmol) of sodium methoxide was added slowly followed by 300 mg (0.617 mmol) of (6S)-1,1,1-trifluoro-6-([(1R,3aR,4S,7aR)-4-hydroxy-7a-methyl-octahydro-inden-1-yl]-6,10-dimethyl-2-trifluoromethyl-undec-3-yne-2,10-diol in 4.0 ml of tetrahydrofurane. Mae reaction mixture was stirred at 80°& for 5 h and then was cooled to 0° C. A 1.0 ml of water, 1.0 ml of 2N NaOH and 20.0 ml of diethyl ether were added. The mixture was stirred at room temp for 30 min, 2.2 g of MgSO₄ was added and mixture was stirred for next 15 min. The suspension was filtrated and solvent evaporated.

The oil residue was chromatographed on columns (100 cm³ and 30 cm³) using dichloromethane:ethyl acetate (4:1) as mobile phase. Fractions containing product were pooled and evaporated to give 279 mg (93%) of product as colorless oil.

¹H NMR (CDCl₃): 6.32 (1H, dt, J=15.7, 7.8 Hz), 5.59 (1H, 15.7 Hz), 4.09 (1H, br s), 2.29 (2H, d, J=7.6 Hz), 2.01 (1H, br d, J=3.3 Hz), 1.86-1-75 (2H, m), 1.63-1.04 (18H, m), 1.21 (6H, s), 1.09 (3H, s), 0.98 (3H, s)

¹³C NMR (CDCl₃): 137.07, 119.81, 71.52, 69.54, 69.57, 57.20, 52.53, 44.16, 43.50, 42.29, 41.43, 40.10, 40.04, 33.39, 29.33, 29.29, 23.01, 22.17, 21.69, 17.86, 17.51, 16.58

### (1R,3aR,4S,7aR)-7a-Methyl-1-[(1S,3E)-6,6,6-trifluoro-5-hydroxy-1-(4-hydroxy-4-methyl-pentyl)-1-methyl-5-trifluoromethyl-hex-3-enyl]-octahydro-inden-4-one (26)

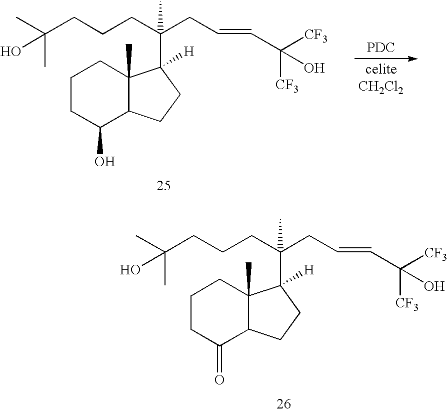

A 25 ml round bottom flask equipped with stir bar and Claisen adapter with rubber septum was charged with 274 mg (0.561 mmol) of (6S,3E)-1,1,1-trifluoro-6-[(1R,3aR,4S,7aR)-4-hydroxy-7a-methyl-octahydro-inden-1-yl]-6,10-dimethyl-2-trifluoromethyl-undec-3-ene-2,10-diol and 10 ml of dichloromethane. A 704 mg (1.871 mmol) of pyridinium dichromate and 740 mg of celite was added and mixture was stirred in room temperature fox 2 h.

The reaction mixture was filtrated through column with silica gel (100 cm³) using dichloromethane: ethyl acetate (4:1) as a mobile phase. The fractions containing product were pooled and evaporated to give 253 mg of yellow oil. The product was used to the next reaction without farther purification.

### (20S)-1,25)-Dihydroxy-20-[(2E)-5,5,5-trifluoro-4-hydroxy-4-trifluoromethyl-pent-2-enyl]-cholecalciferol (3)

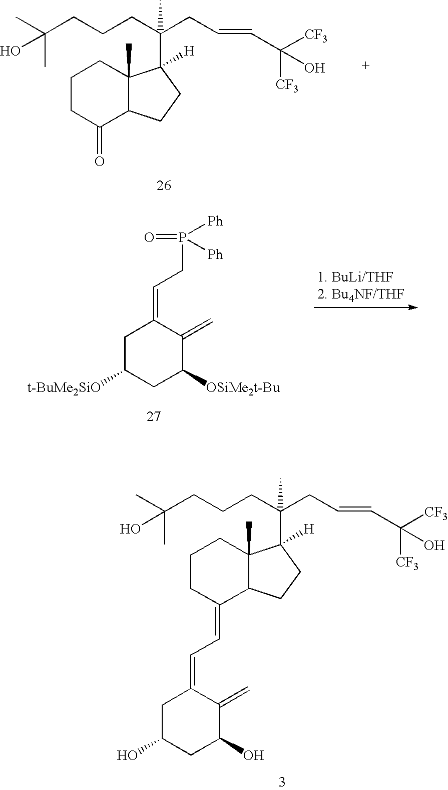

A 25 ml round bottom flask equipped with stir bar and Claisen adapter with rubber septum was charged with 1,765 g (3.028 mmol) of (1S,5R)-1,5-bis-((tert-butyldimethyl)silanyloxy)-3-[2-(diphenylfosphinoyl)-eth-(Z)-ylidene]-2-methylene-cyclohexane and 10.0 ml of tetrahydrofurane. The reaction mixture was cooled to −78° C. and 1.8 ml (2.88 mmol) of 1.6M n-butyllithium in tetrahydrofurane was added dropwise. The resulting deep red solution was stirred at −78° C. for 20 min and 253 mg (0.520 mmol) of (1R,3aR,4S,7aR)-7a-methyl-1-[(1S,3E)-6,6,6-trifluoro-5-hydroxy-1-(4-hydroxy-4-methyl-pentyl) 1-methyl-5-trifluoromethyl-hex-3-enyl]-octahydro-inden-4-one was added dropwise in 1.5 ml of tetrahydrofurane. The reaction mixture was stirred for 5 h (last 0.5 h at −20° C.) and then the bath was removed and the mixture was poured into 50 ml of ethyl acetate and 100 ml of brine. The water fraction was extracted three times with 50 ml of ethyl acetate, dried over Na₂SO₄ and evaporated.

The oil residue was chromatographed on column (60 cm³, protected from light) using hexane:ethyl acetate (4:1) as mobile phase. Fractions containing product were pooled and evaporated to give colorless oil (304 mg) which was treated with 5 ml of 1M tetrabutylammonium fluoride in tetrahydrofurane. The reaction mixture was stirred at room temperature for 21 h.

The mixture was dissolved by the addition of 150 ml of ethyl acetate and extracted six times with 50 ml of water:brine (1:1) and 50 ml of brine, dried over Na₂SO₄ and evaporated.

The oil residue was chromatographed on column (50 cm³, protected from light) using ethyl acetate as mobile phase. Fractions containing product were pooled and evaporated to give product as colorless oil, Oil was dissolved in methyl acetate and evaporated (4 times) to give 176 mg (54%, two steps) of product as white foam.

[α]_{D}²⁹=−4.5° c=0.331 CHCl₃

UV λmax (EtOH) 204.50 nm (ε 17846), 266.17 nm (ε 16508)

¹H NMR (CDCl₃): 6.36 (1H, d, J=11.3 Hz), 6.32 (1H, dt, J=15.1, 7.5 Hz), 6.00 (1H, d, J=11.1 Hz), 5.59 (1H, d, J=15.8 Hz, 5.33 (1H, s), 4.99 (1H, s), 4.53 (1H, brs), 4.43 (1H, dd, J=7.7, 4.3 Hz), 4.25-4.00 (1H, nm), 2.81 (1H, dd, J=12.1, 3.8 Hz), 2.59 (1H, dd, J=13.3, 2.9 Hz), 2.34-0.29 (3H, m), 2.05-1.96 (3H, m), 1.93-1.87 (1H, m), 1.71-1.21 (17H, m), 1.21 (6H, s), 1.12-1.05 (1H, m), 0.95 (3H, s), 0.66 (3H, s)

¹³C NMR (CDCl₃): 147.48; 142.53, 136.92, 133.05, 124.83, 122-39 (q, J=141.5 Hz), 119.76, 117.58, 117.49, 111, 71, 71.61, 70.73, 66.90, 57.39, 56.62, 46.79, 45.18, 43.99, 42.83, 42.48, 41.29, 40.13, 40.04, 29.62, 29.28, 28.98, 23.50, 23.06, 22.24, 21.90, 17.74, 15.11

| | | | | |
|---|---|---|---|---|
| MS HRES | Calculated for: | C₃₃H₄₈F₆O₄ | [M + Na]⁺ | 645.3349 |
| | Observed: | | [M + Na]⁺ | 645.3346 |

### Example 5

### Synthesis of (20R)-1,25-Dihydroxy-20-(5,5,5-trifluoro-4-hydroxy-4-trifluoromethyl-pent-2-ynyl)-cholecalciferol (4)

### (3R)-3-[(1R,3aR,4S,7aR)-4-(tert-Butyl-dimethyl-silanyloxy)-7a-methyl-octahydro-inden-1-yl]-7-hydroxy-3,7-dimethyl-octanal (30)

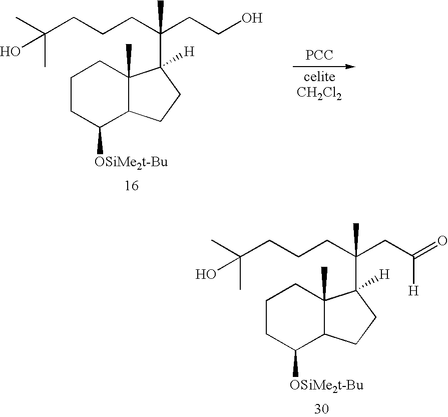

A 50 ml round bottom flask equipped with stir bar and Claisen adapter with rubber septum was charged with 1.558 g (7.228 mmol) of pyridinium chlorochromate, 1.60 g of celite and 20 ml of dichloromethane. A 1.440 g (3.267 mmol) of (3R)-3-[(1R, 3aR,4S,7aR)-4-tert-butyl-dimethyl-silanyloxy)-7a-dimethyl-octahydro-inden-1-yl]-3,7-dimethyl-octane-7-diol in 10 ml of dichloromethane was added dropwise and mixture was stirred in room temperature for 2 h 50 min.

The reaction mixture was filtrated through column with silica gel (75 cm³) and celite (2 cm) and using dichloromethane, dichloromethane:ethyl acetate (4:1) as a mobile phase. The fractions containing product were pooled and evaporated to give 1.298 g of yellow oil. The product was used to the next reaction without farther purification.

### (6R)-6-[(1R,3aR,4S,7aR)-4-(tert-Butyl-dimethyl-silanyloxy)-7a-methyl-octahydro-inden-1-yl]-2,6-dimethyl-non-8-yn-2-ol (31)

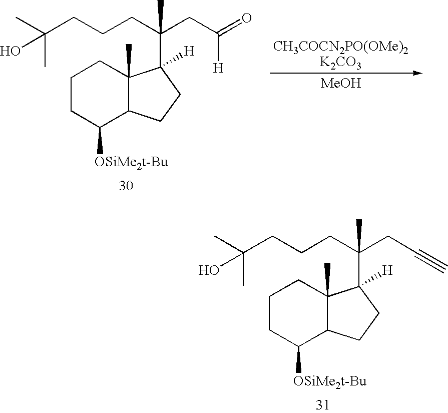

A 50 ml round bottom flask equipped with stir bar and Claisen adapter with rubber septum was charged with 1.298 g (2.958 mmol) of (3R)-3-[(1R,3aR,4S,7aR)-4-(tert-butyl-dimethyl-silanyloxy)-7a-meth-yl-octahydro-inden-1-yl]-7-hydroxy-3,7-dimethyl-octanal and 30 ml of methanol. A 1.137 g (5.916 mmol) of 1-diazo-2-oxo-propyl)-phosphonic acid dimethyl ester in 3 ml of methanol was added and the resulting mixture was cooled in an ice bath to 0° C. A 1.140 g (8.248 mmol) of potassium carbonate was added and the reaction mixture was stirred in the ice bath for 30 min and then at room temperature for 2 h 50 min.

A 100 ml of water was added and the mixture was extracted three times with 80 ml of ethyl acetate, dried over Na₂SO₄ and evaporated.

The oil residue was chromatographed on column (200 cm³) using hexane:ethyl acetate (7:1) as mobile phase. Fractions containing product were pooled and evaporated to give 1.151 g (81%) of product as colorless oil.

[α]_{D}¹⁹=+18.3° c=0.54 CHCl₃

¹H NMR (CDCl₃): 3.99 (1H, br s), 2.16-2.07 (2H, m), 2.00-1.97 (1H, m), 1.92 (1H, t, J=2.6 Hz), 1.84-1.74 (1H, m), 1.67-1.64 (1H, m), 1.58-1.22 (16H, m), 1.22 (6H, s), 1.04 (3H, s), 0.99 (3H, s), 0.88 (9H, s), 0.00 (3H, s), −0.01 (3H, s)

| | | | | |
|---|---|---|---|---|
| MS HRES | Calculated for: | C₂₇H₅₀O₂Si | [M + Na]⁺ | 457.3472 |
| | Observed: | | [M + Na]⁺ | 457.3473 |

### (1R,3aR,4S,7aR)-4-(tert-Butyl-dimethyl-silanyloxy)-1-[(1R)-1,5-dimethyl-1-prop-2-ynyl-5-trimethylsilanyloxy-hexyl]-7a-methyl-octahydro-indene (32)

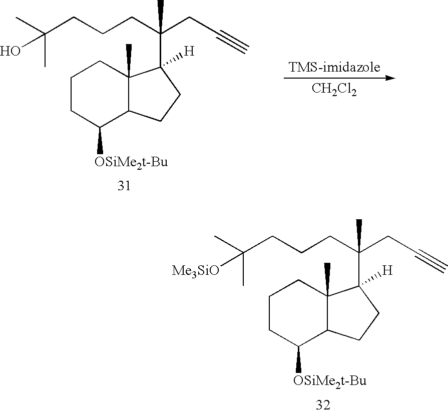

A 50 ml round bottom flask equipped with stir bar and Claisen adapter with rubber septum was charged with 1.151 g (2.647 mmol) of (6R)-6-[(1R,3aR,4S,7aR)-4-(tert-butyl-dimethyl-silanyloxy)-7a-methyl-octahydro-inden-1-yl]-2,6-dimethyl-non-8-yn-2-ol and 20 ml of dichloromethane. A 2.0 ml (13.63 mmol) of 1-(trimethylsilyl)imidazole was added dropwise. The mixture was stirred at room temperature for 1 h.

A 100 ml of water was added and the mixture was extracted three times with 50 ml of ethyl acetate, dried over Na₂SO₄ and evaporated.

The oil residue was chromatographed on column (75 cm³) using hexane:ethyl:acetate (25:1) as mobile phase. Fractions containing product were pooled and evaporated to give 1.260 g (94%) of product as colorless oil.

[α]_{D}²⁹=18.5° c=0.46, CHCl₃

¹H NMR (CDCl₃): 3.98 (1H, br s), 2.12-2.08 (2H, m), 20.5-1.95 (2H, m), 1.92-1.90 (1H, m), 1.83-1.21 (16H, m), 1.21 (6H, s), 1.04 (3H, s), 0.98 (3H, s), 0.88 (9H, s), 0.11 (9H, s), 0.00 (3H, s), −0.01 (3H, s)

¹³C NMR(CDCl₃) 83.00, 74.07, 69.70, 69.50, 56.63, 53.03, 45.66, 43.74, 41.35, 39.59, 39.45, 34.38, 29.99, 29.60, 25.85, 22.81, 22.43, 22.06, 18.56, 18.05, 17.76, 16.49, 2.65, −4.77, −5.13

| | | | | |
|---|---|---|---|---|
| MS HRES | Calculated for: | C₃₀H₅₈O₂Si₂ | [M + Na]⁺ | 529.3867 |
| | Observed: | | [M + Na]⁺ | 529.3868 |

### (6R)-6-[(1R,3,aR,4S,7a R)-4-(tert-Butyl-dimethyl-silanyloxy)-7a-methyl-octahydro-inden-1-yl]-1,1,1-trifluoro-6,10-dimethyl-2-trifluoromethyl-10-trimethylsilanyloxy-undec-3-yn-2-ol (33)

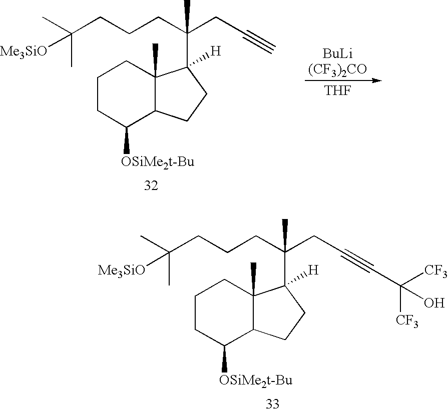

A two neck 50 ml round bottom flask equipped with stir bar, Claisen adapter with rubber septum and funnel (with cooling bath) was charged with 1.252 g (2.470 mmol) of (1R,3aR,4S,7aR)-4-(tert-butyl-dimethyl-silanyloxy)-4-[(1R)-1,5-dimethyl-1-prop-2-ynyl-5-trimethylsilanyloxy-hexyl]-7a-methyl-octahydro-indene and 25 ml of tetrahydrofurane. The funnel was connected to container with hexafluoroacetone and cooled (acetone, dry ice). The reaction mixture was cooled to −70° C. and 2.4 ml (3.84 mmol) of 1.6M n-butyllithium in tetrahydrofurane was added dropwise. After 30 min hexafluoroacetone was added (the container's valve was opened three times). The reaction was stirred at −70° C. for 2 h then 5.0 ml of saturated solution of ammonium chloride was added.

The mixture was dissolved by the addition of 100 ml of saturated solution of ammonium chloride and extracted three times with 80 ml of ethyl acetate, dried over Na₂SO₄ and evaporated.

The residue was chromatographed twice on columns (75 cm³) using hexane:ethyl:acetate (10:1) as mobile phase to give 0.711 g of mixture of product and polymer (from hexafluoroacetone).

### (6R)-1,1,1-Trifluoro-6-[(1R,3aR,4S,7aR)-4-hydroxy-7a-methyl-octahydro-inden-1-yl]-6,10-dimethyl-2-trifluoromethyl-undec-3-yne-2,10-diol (34)

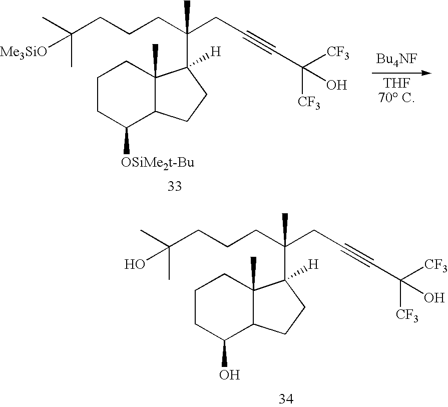

A 25 ml round bottom flask equipped with stir bar and Claisen adapter with rubber septum was charged with crude (ca 2.470 mmol) (6R)-6-[(1R,3aR,4S,7aR)-4-(tert-butyl-dimethyl-silanyloxy)-7a-methyl-octahydro-inden-1-yl]-1,1,1-trifluoro-6, 10-dimethyl-2-trifluoromethyl-10-trimethylsilanyloxy-undec-3-yn-2-ol and 15.0 ml (15.0 mmol) of 1M tetrabutylammonium fluoride in tetrahydrofurane. The reaction mixture was stirred at 70° C. for 96 h.

The mixture was dissolved by the addition of 150 ml of ethyl acetate and extracted six times with 50 ml of water:brine (1.1) and 50 ml of brine, dried over Na₂SO₄ and evaporated. The oil residue was chromatographed on columns, 200 cm³ and 75 cm³ using hexane: ethyl acetate (2:1). The fractions containing product were pooled and evaporated to give 979 mg (81%) of product as colorless oil.

[α]_{D}³⁰=+1.04° c=0.48, CHCl₃

¹H NMR (CDCl₃): 4.08 (1H, br s), 2.24 (1H, AB, J=17.2 Hz), 2.17 (1H, AB, J=17.2 Hz), 2.05-2.02 (1H, m), 1.85-1.76 (2H, m), 1.66-1.20 (18H, m), 1.26 (3H, s), 1.25 (3H, s), 1.07 (3H, s), 1.01 (3H, s)

| | | | | |
|---|---|---|---|---|
| MS HRES | Calculated for: | C₂₄H₃₆F₆O₃ | [M + Na]⁺ | 509.2461 |
| | Observed: | | [M + Na]⁺ | 509.2463 |

### (1R,3aR,4S,7aR)-7a-Methyl-1-[(1R)-6,6,6-trifluoro-5-hydroxy-1-(4-hydroxy-4-methyl-pentyl)-1-methyl-5-trifluoromethyl-hex-3-ynyl]-octahydro-inden-4-one (35)

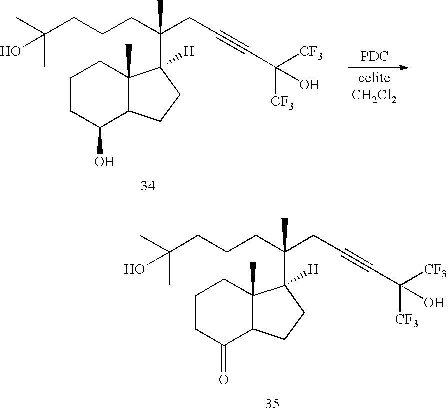

A 25 ml round bottom flask equipped with stir bar and Claisen adapter with rubber septum was charged with 291 mg (0.598 mmol) of (6R)-1,1,1-trifluoro-6-[(1R, 3aR,4S,7aR)-4-hydroxy-7a-methyl-octahydro-inden-1-yl]-6,10-dimethyl-2-trifluoromethyl-undec-3-yne-2,10-diol and 10 ml of dichloromethane. A 700 mg (1.861 mmol) of pyridinium dichromate and 720 mg of celite was added and mixture was stirred in room temperature for 3 h.

The reaction mixture was filtrated through column with silica gel (75 cm³) using dichloromethane, dichloromethane:ethyl acetate (4:1, 3:1) The fractions containing product were pooled and evaporated to give 271 mg (94%) of product as yellow oil.

### (20R)-1,25-Dihydroxy-20-(5,5,5-trifluoro-4-hydroxy-4-trifluoromethyl-pent-2-ynyl)-cholecalciferol (4)

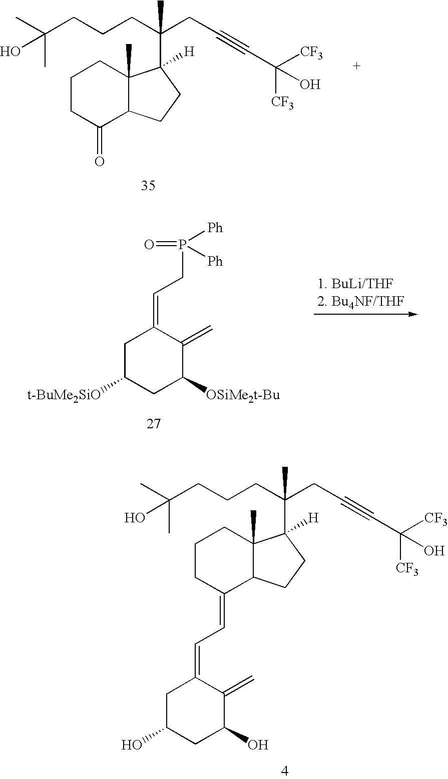

A 25 ml round bottom flask equipped with stir bar and Claisen adapter with rubber septum was charged with 2.118 g (3.634 mmol) of (1S,5R)-1,5-bis-((tert-butyldimethyl)silanyloxy)-3-[2-(diphenylfosphinoyl)-eth-(Z)-ylidene]-2-methylene-cyclohexane and 10 ml of tetrahydrofurane. The reaction mixture was cooled to −78° C. and 2.2 ml (3.52 mmol) of 1.6M n-butyllithium in tetrahydrofurane was added dropwise. The resulting deep red solution was stirred at −78° C. for 20 min and 271 mg, (0.559 mmol) of (1R,3aR,4S,7aR)-7a-methyl-1-[(1R,3E)-6,6,6-trifluoro-5-hydroxy-1-(4-hydroxy-4-methyl-pentyl)-1-methyl-5-trifluoromethyl-hex-3-ynyl]-octahydro-inden-4-one was added dropwise in 1.5 ml of tetrahydrofurane. The reaction mixture was stirred at −78° C. for 5 h and then the bath was removed and the mixture was poured into 100 ml of saturated solution of ammonium chloride and extracted three times with 50 ml of ethyl acetate, dried over Na₂SO₄ and evaporated.

The oil residue was chromatographed on column (50 cm³, protected from light) using hexane:ethyl acetate (4:1) as mobile phase. The fractions contains impurities was chromatographed on column (50 cm³, protected from light) using hexane:ethyl acetate (5:1) as mobile phase. Fractions containing product were pooled and evaporated to give colorless oil (250 mg) which was treated with 5 ml of 1M tetrabutylammonium fluoride in tetrahydrofurane. The reaction mixture was stirred at room temperature for 18 h.

The mixture was dissolved by the addition of 150 ml of ethyl acetate and extracted six times with 50 ml of water:brine (1:1) and 50 ml of brine, dried over Na₂SO₄ and evaporated.

The oil residue was chromatographed on column (50 cm³, protected from light) using ethyl acetate as mobile phase. Fractions containing product were pooled and evaporated to give product as colorless oil. Oil was dissolved in methyl acetate and evaporated (4 times) to give 194 mg (56%) of product as white foam.

[α]_{D}³⁰=+7.9° c=0.38, EtOH

UV λmax (EtOH): 212.33 nm (ε 14113), 265.00 nm (ε 15960)

¹H NMR (D6-DMSO): 8.93 (1H, s), 6.18 (1H, d, J=11.3 Hz), 5.96 (1H, d, J=11.3 Hz), 5.22 (1H, s), 4.86 (1H, d, J=4.83 Hz), 4.75 (1H, s), 4.54 (1H, d, J=3.63 Hz), 4.20-4.15 (1H, m), 4.06 (1H, s), 3.98 (1H, br s), 2.77 (1H, d, J=13.7 Hz), 2.40-2.33 (1H, m), 2.27-2.14 (3H, m), 2.00-1.90 (2H, m), 1.82-1.78 (2H, m), 1.64-1.54 (5H, m), 1.47-1.18 (101H, m), 1.05 (3H, s), 1.05 (3H, s), 0.95 (3H, s), 0.59 (3H, s)

¹³C NMR (D6-DMSO): 149.38, 139.51, 135.94, 122.32, 121.47 (q, J=142.9 Hz), 117.99, 109.77, 89.53, 70.58, 68.72, 68.35, 65.06, 56.02, 55.91, 46.06, 44.85, 44.65, 43.11, 29.30, 29.03, 28.78, 28.32, 23.05, 22.40, 21.90, 21.52, 18.27, 14.29

| | | | | |
|---|---|---|---|---|
| MS HRES | Calculated for: | C₃₃H₄₆F₆O₄ | [M + Na]⁺ | 643.3192 |
| | Observed: | | [M + Na]⁺ | 643.3190 |

### Example 6

### Synthesis of (20R)-1,25-Dihydroxy-20-[(2E)-5,5,5-trifluoro-4-hydroxy-4-trifluoromethyl-pent-2-enyl]-cholecalciferol (5)

### (3E,6R)-1,1,1-Trifluoro-6-[(1R,3aR,4S,7aR-4-hydroxy-7a-methyl-octahydro-inden-1-yl]-6,10-dimethyl-2-trifluoromethyl-undec-3-ene-2,10-diol (36)

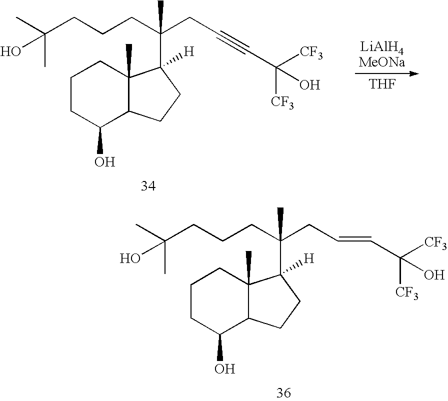

A 25 ml round bottom flask equipped with stir bar and condenser with nitrogen sweep was charged with 4.5 ml (4.5 mmol) of 1M lithium aluminum hydride in tetrahydrofurane and the mixture was cooled to 0° C. A 243 mg (4.50 mmol) of sodium methoxide was added slowly followed by substrate 337 mg (0.693 mmol) of (3E,6R)-1,1,1-trifluoro-6-[(1R,3aR,4S,7aR)-4-hydroxy-7a-methyl-octahydro-inden-1-yl]-6,10-dimethyl-2-trifluoromethyl-undec-3-yne-2,10-diol in 5 ml of tetrahydrofurane. The reaction mixture was stirred at 80° C. for 6 h 30 min and then was cooled to 0° C. A 1 ml of water, 1 ml of 2N NaOH and 20 ml of diethyl ether were added. The mixture was stirred at room temp for 30 min and 2.2 g of MgSO₄ was added and mixture was stirred for next 15 min. The suspension was filtrated and solvent evaporated.

The oil residue was chromatographed on column (00 cm³) using dichloromethane:ethyl acetate (4:1) as mobile phase. Fractions containing product were pooled and evaporated to give 330 mg (97%) of product as colorless oil.

¹H NMR (CDCl₃): 6.28 (1H, dt, J=15.7, 7.3 Hz), 5.59 (1H, d, J=15.4 Hz), 6.12 (1H, br s), 2.12 (2H, d, J=7.7 Hz), 2.06-1.98 (1H, m), 1.85-1.74 (2H, m), 1.68-1.16 (18H, m), 1.22 (6H, s), 1.08 (3H, s), 0.98 (3H, s)

### (1R,3aR,4S,7a)-7a-Methyl-1-[(1R,3E)-6,6,6-trifluoro-5-hydroxy-1-(4-hydroxy-4-methyl-pentyl)-1-methyl-5-trifluoromethyl-hex-3-enyl]-octahydro-inden-4-one (37)

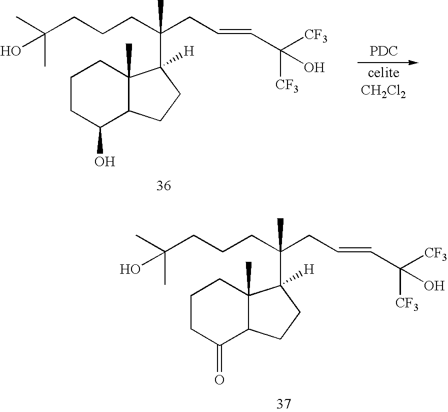

A 25 ml round bottom flask equipped with stir bar and Claisen adapter with rubber septum was charged with 330 mg (0.675 mmol) of (3E,6Z)-1,1,1-trifluoro-6-[(1R,3aR,4S,7aR)-4-hydroxy-7a-methyl-octahydro-inden-1-yl]-6,10-dimethyl-2-trifluoromethyl-undec-3-ene-2,10-diol and 10 ml of dichloromethane. A 920 mg (2.445 mmol) of pyridinium dichromate was added and mixture was stirred in room temperature for 7 h.

The reaction mixture was filtrated through column with silica gel (60 cm³) using dichloromethane ethyl acetate (4:1) as mobile phase. The fractions containing product were pooled and evaporated to give 302 mg (92%) of product as colorless oil.

[α]_{D}³⁰=−17.7° c=0.46, CHCl₃

¹H NMR (CDCl₄): 6.30 (1H, dt, J=15.6, 7.7 Hz), 5.60 (1H, d, J=15.6 Hz), 2.40 (1H, dd, J=11.1, 7.3 Hz), 2.30-2.14 (6H, m), 2.06-1.98 (1H, m), 1.96-1.81 (1H, m), 1.78-1.30 (13H, m), 1.24 (3H, s), 1.23 (3H, s), 0.98 (3H, s), 0.74 (3H, s)

¹³C NMR (CDCl₃): 212.12, 136.27, 120.28, 71.45, 62.27, 57.44, 50.69, 44.28, 42.02, 40.76, 40.17, 39.69, 39.65, 29.34, 29.23, 23.98, 22.66, 22.24, 18.67, 18.19, 15.47

| | | | | |
|---|---|---|---|---|
| MS HRES | Calculated for: | C₂₄H₃₆F₆O₃ | [M + Na]⁺ | 509.2461 |
| | Observed: | | [M + Na]⁺ | 509.2463 |

(1R,3aR,4S,7aR)-7a-Methyl-1-[(1R,3E)-6,6,6-trifluoro-1-methyl-1-(4-methyl-4-trimethylsilanyloxy-pentyl)-5-trifluoromethyl-5-trimethylsilanlyloxy-hex-3-enyl]-octahydro-inden-4-one (38)

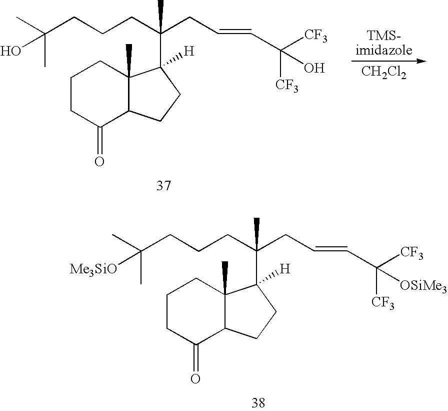

A 25 ml round bottom flask equipped with stir bar and Claisen adapter with rubber septum was charged with 292 mg (0.600 mmol) of (1R,3aR,4S,7aR)-7a-methyl-1-[(1R,3E)-6,6,6-trifluoro-5-hydroxy-1-(4-hydroxy-4-methyl-pentyl)-1-methyl-5-trifluoromethyl-hex-3-enyl]-octahydro-inden-4-one and 8 ml of dichloromethane. A 0.7 ml (4.8 mmol) of 1-(trimethylsilyl)imidazole was added dropwise. The mixture was stirred at room temperature for 2 h.

A 100 mil of water was added and the mixture was extracted three times with 50 ml of ethyl acetate, dried over Na₂SO₄ and evaporated.

The oil residue was chromatographed on column (60 cm³) using hexane:ethyl:acetate (10:1, 4:1) as, mobile phase. Fractions containing product were pooled and evaporated to give 360 mg (95%) of product as colorless oil.

### (20R)-1,25-dihydroxy-20-[(2E)-5,5,5-trifluoro-4-hydroxy-4-trifluoromethyl-pent-2-enyl]-cholecalciferol (5)

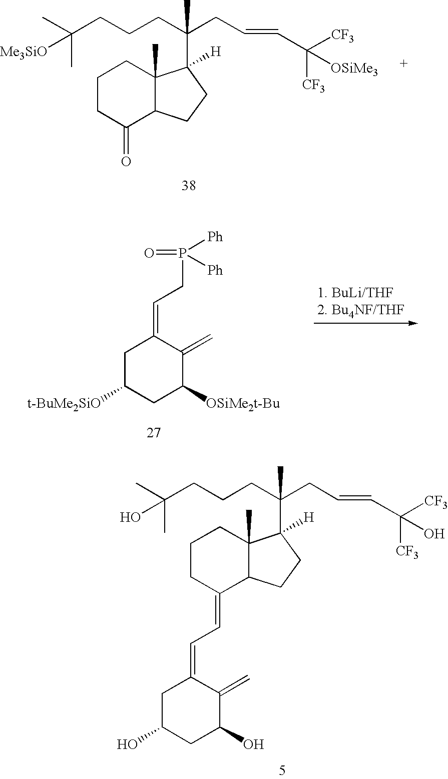

A 25 ml round bottom flask equipped with stir bar and Claisen adapter with rubber septum was charged with 760 mg (1.304 mmol) of (1S,5R)-1,5-bis-((tert-butyldimethyl)silanyloxy)-3-[2-(diphenylfosphinoyl)-eth-(Z)-ylidene]-2-methylene-cyclohexane and 1000 ml of tetrahydrofurane. The reaction mixture was cooled to −78° C. and 0.8 ml (1.28 mmol) of 1.6M n-butyllithium in tetrahydrofurane was added dropwise. The resulting deep red solution was stirred at −78° C. for 20 min and 358 mg (0.567 mmol) of (1R,3aR,4S,7aR)-7a-methyl-[(1R,3E)-6,6,6-trifluoro-1-methyl-1-(4-methyl-4-trimethylsilanyloxy-pentyl)-5-trifluoromethyl-5-trimethylsilanyloxy-hex-3-enyl]-octahydro-inden-4-one was added dropwise in 1.5 ml of tetrahydrofurane. The reaction mixture was stirred for 4 h (last 0.5 h at −20° C.) and then the bath was removed and the mixture was poured into 50 ml of ethyl acetate and 100 ml of brine. The water fraction was extracted three times with 50 ml of ethyl acetate, dried over Na₂SO₄ and evaporated.

The oil residue was chromatographed on column (50 cm³, protected from light) rising hexane:ethyl acetate (10:1) as mobile phase. Fractions containing product and some mono deprotected compound were pooled and evaporated to give colorless oil (440 mg) which was treated with 10 ml of 1M tetrabutylammonium fluoride in tetrahydrofurane. The reaction mixture was stirred at room temperature for 21 h.

The mixture was dissolved by the addition of 150 ml of ethyl acetate and extracted six times with 50 ml of water:brine (1:1) and 50 ml of brine, dried over Na₂SO₄ and evaporated.

The oil residue was chromatographed on column (50 cm³, protected from light) using ethyl acetate as mobile phase. Fractions containing product were pooled and evaporated to give 30 5 mg (86%, two steps) of product as colorless solid.

[α]_{D}³¹=+13.4° c=0.44, EtOH

UV λmax (EtOH): 212.76 nm (ε 15453), 265.03 (ε 17341)

¹H NMR (D6-DMSO): 8.04 (11H, s), 6.28 (1H, dt, J−15.5, 7.6 Hz), 6.18 (1H, d, J=11.1 Hz), 5.97 (1H, d, J=11.1), 5.61 (1H, d, J=15.5 Hz), 5.22 (1H, s), 4.75 (1H, s), 4.19-4.16 (1H, m), 3.98 (1H, br s), 2.77 (1H, d, 13.9 Hz), 2.35 (1-1, d, J=11.7 Hz), 2.16 (1H, dd, J=13.6, 5.3 Hz), 2.07 (2H, d, J=7.31 Hz), 1.99-1.90 (2H, m), 1.81-1.78 (1H, m), 1.64-1.55 (6H, m), 1.48-117 (12H, m), 1.05 (6H, s), 0.90 (3H, s), 0.84 (1H, s), 0.61 (3H, s)

¹³C NMR (DMSO): 149.34, 139.65, 136.40, 135.82, 122.60 (q, J=143.0 Hz), 122.32, 19.80, 117.90, 109.76, 68.68, 68.36, 65.04, 56.35, 56.00, 46.18, 44.85, 44-64, 43.09, 41.05, 40.42, 29.34, 29.12, 28.31, 23.08, 22.47, 21.79, 21.58, 17.91, 14.57

| | | | | |
|---|---|---|---|---|
| MS HRES | Calculated for: | C₃₃H₄₈F₆O₄ | [M + Na]⁺ | 645.3349 |
| | Observed: | | [M + Na]⁺ | 645.3355 |

### Example 7

### Determination of Maximum Tolerated Dose (MTD) of Vitamin D₃ Analogs

The maximum tolerated dose of the vitamin D₃ compounds of the invention were determined in eight week-old female C57BL/6 mice (3 mice/group) dosed orally (0.1 ml/mouse) with various concentrations of Vitamin D₃ analogs daily for four days. Analogs were formulated in miglyol for a final concentration of 10, 30, 100 and 300 μg/kg when given at 0.1 ml/mouse p.o. daily. Blood for serum calcium assay was drawn by tail bleed on day five, the final day of the study. Serum calcium levels were determined using a colormetric assay (Sigma Diagnostics, procedure no. 597). The highest dose of analog tolerated without inducing hypercalcemia (serum calcium>10.7 mg/dl) was taken as the maximum tolerated does (MTD). Table 1 shows the relative MTD for vitamin D₃ compounds.

TABLE 1

| | MTD | |
|---|---|---|
| | (mice) | IFN-γ |
| COMPOUND | μg/kg | IC₅₀ pM |
| (20S)-1,25-Dihydroxy-20-(5,5,5-trifluoro-4- | 0.3 | 49.0 |
| hydroxy-4-trifluoromethyl-pent-2- | | |
| ynyl)cholecalciferol (1) | | |
| (20S)-1,25-Dihydroxy-20-(5,5,5-trifluoro-4- | 0.3 | 42.0 |
| hydroxy-4-trifluoromethyl-pent-(2Z)- | | |
| enyl)cholecalciferol (2) | | |
| (20S)-1,25-Dihydroxy-20-[(2E)-5,5,5-trifluoro-4- | 0.03 | 44.0 |
| hydroxy-4-trifluoromethyl-pent-2-enyl]- | | |
| cholecalciferol (3) | | |
| (20R)-1,25-Dihydroxy-20-(5,5,5-trifluoro-4- | 0.03 | 38.0 |
| hydroxy-4-trifluoromethyl-pent-2-ynyl)- | | |
| cholecalciferol (4) | | |
| (20R)-1,25-Dihydroxy-20-[(2E)-5,5,5-trifluoro-4- | 0.1 | 49.0 |
| hydroxy-4-trifluoromethyl-pent-2-enyl]- | | |
| cholecalciferol (5) | | |
| (20S)-1α-Fluoro-25-hydroxy-20-(5,5,5-trifluoro-4- | 100 | 358.3 |
| hydroxy-4-trifluoromethyl-pent-2-ynyl)- | | |
| cholecalciferol (6) | | |

### Example 8

### Immunological Assay of Vitamin D₃ Compounds

Immature dendritic cells (DC) were prepared as described in Romani, N. et al., J. Immunol. Meth. 196.137. IFN-γ production by allogeneic T cell activation in the mixed leukocyte response (MLR) was determined as described in Penna, G., et al., J. Immunol., 164: 2405-2411 (2000).

Briefly, peripheral blood mononuclear cells (PBMC) were separated from buffy coats by Ficoll gradient and the same number (3×105) of allogeneic PBMC from 2 different donors were co-cultured in 96-well flat-bottom plates. After 5 days, IFN-γ production in the MLR assay was measured by ELISA and the results expressed as amount (nM) of test compound required to induce 50% inhibition of IFN-γ production (IC₅₀) (Table 1).

### Example 9

### Osteoporosis—Early Curative Treatment

Animals

Three-month old Sprague Dawley female rats were purchased from CERJ or Charles River, France. Rats underwent bilateral ovariectomy (OVX) or sham-operation (Sham) under anesthesia with intraperitoneal ketamine hydrochloride (50 mg/kg BW). The success of ovariectomy was evidenced at necropsy by weighing the uterus and visualizing the absence of ovarian tissue.

Throughout the whole experiment, rats were housed at 22±2° C. with a 12 h:12 h light-dark cycle. The animals were pair-fed a standard diet (Safe, 0.6% P, 0.8% Ca) and received Eau de Volvic ad libitum.

Experimental procedures were approved by the Animal Ethics Committee of Prostrakan and by DDSV of Seine St Denis, France.

The treatment started 3 weeks post-ovariectomy. Compounds were firstly dissolved in ethanol (1 mg/ml). The other dilutions were done in Miglyol 812N. Compounds or vehicle (sham, OVX control rats) were given by daily oral gavage (5 ml/kg), 5/7 days for 3 weeks. Just before operation a group of intact rats were sacrificed for baseline parameters. Before the beginning of treatment, groups of sham and OVX rats were sacrificed to serve as basal controls. Ten and three days before sacrifice, rats were given subcutaneous injections of calcein (10 ml/kg, Sigma) to determine dynamic changes in bone tissue. The day before sacrifice, the rats were fasted and housed in metabolic cages to collect overnight urines.

Evaluation of Bone Parameters: pQCT, μCT DEXA Analysis

At necropsy, right long bones were removed and fixed in 70% ethanol for further μCT (tibia and femur) and histomorphometry (tibia) analysis. The whole left legs were collected for pQCT analysis. The fourth and fifth lumbar vertebrae were dissected for DXA analysis. Excised tibias were scanned by a μCT machine (Scanco Medical) with software version 3.1 for a 2D-evaluation. The scans started at a distance of 1 mm of the reference line in the proximal tibia metaphysis. Five 0.5 mm-spaced slices were analyzed. The slice thickness was 20-30 μm. Bone volume (BV/TV) and trabeculae number (TbN) and thickness (TbTh) were assessed. Excised tibias were scanned by a pQCT machine (Stratec XCT Research SA+) with software version 5.4 to assess trabecular and cortical Bone Mineral Density (BMD) of proximal tibia metaphysis. For analysis of trabecular bone, the distance between the reference line and the first measurement line was 3 mm. Three other lines separated by 1 mm were analyzed. The cortical BMD was analyzed at 20 mm from the reference line. The voxel size was 0.10 mm. The peel mode used was 20/50. The BMD of excised fourth (L4) and fifth (L5) lumbar vertebrae were scanned by a DXA machine (Hologic QDR 4500) with software optimized for small animal studies. The regional high-resolution software selected a thin X-ray aperture. The large region was 68-71 and the narrow one was 35-21 to assess the body of the vertebrae of L4 and L5.

### Histomorphometry Analysis

Left tibia from each rat was removed and dissected free of adjacent tissues. The bones were fixed in 70% ethanol, dehydrated in graded concentrations of ethanol, defatted in xylene, then embedded without decalcification in methyl methacrylate. Five μm-thick sections were made and stained with toluidine blue and cyanin solochrome and used for structural and cellular parameters evaluation. Ten μm-thick sections remained unstained for florescence microscopy observations. Structural and dynamic parameters were measured in the secondary spongiosa of the proximal tibial metaphysis situated about 1 mm distal from the growth plate-epiphyseal junction. Structural parameters, i.e. trabecular bone volume BV/TV (%), trabecular thickness (μm), connections and number, as well as cellular parameters (osteoblast, osteoid surfaces and osteoclast number), and dynamic parameters (mineralizing surface, mineral apposition rate, bone formation rate) were evaluated.

### Serum and Urinary Biochemistry

Osteocalcin (IRMA kit, Immutopics), DPyr (Metra DPD EIA kit), CTx (Ratlaps ELISA, Nordic, Bioscience Diagnostics) Ca, P, creatinin (Cobas Mira analyser) and rat PTH (Immutopics) were assessed in serum or urine material according to the manufacturer's instructions.

### Results

Data were expressed as mean±sem. Statistics were calculated using StatView version 5.0 for Windows (SAS Institute Inc.). The ANOVA test was used for all groups. Significant difference between groups was determined by Student's t-test. p<0.05 or lower was considered a significant difference.

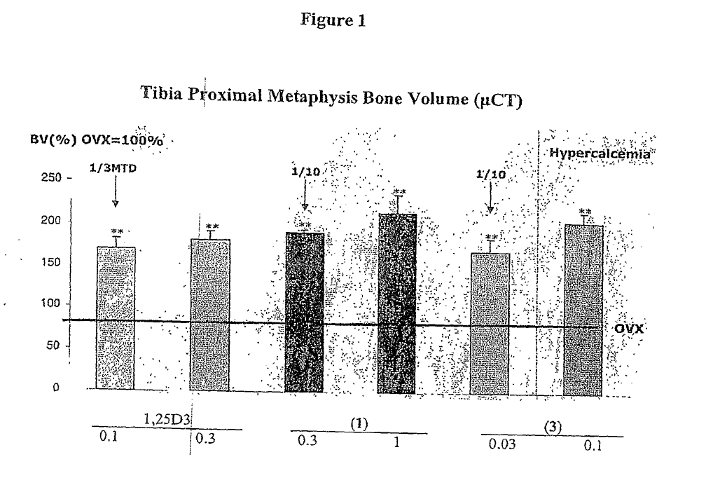

FIG. 1 shows tibia proximal metaphysic bone volume (μCT) measurements in 3 month old OVX rats.

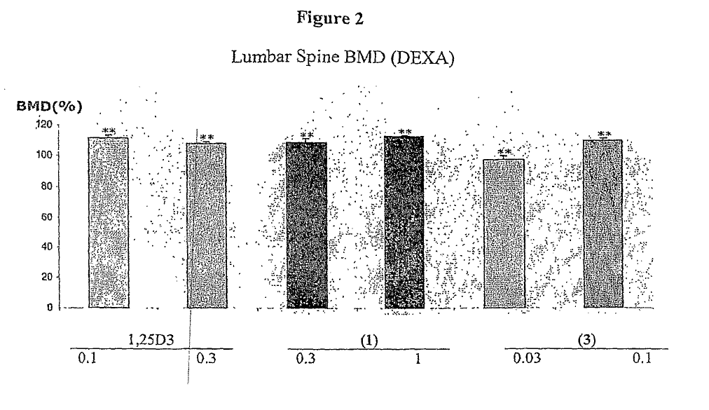

FIG. 2 shows lumbar spine BMD (DEXA) measurements in 3 month old OVX rats.

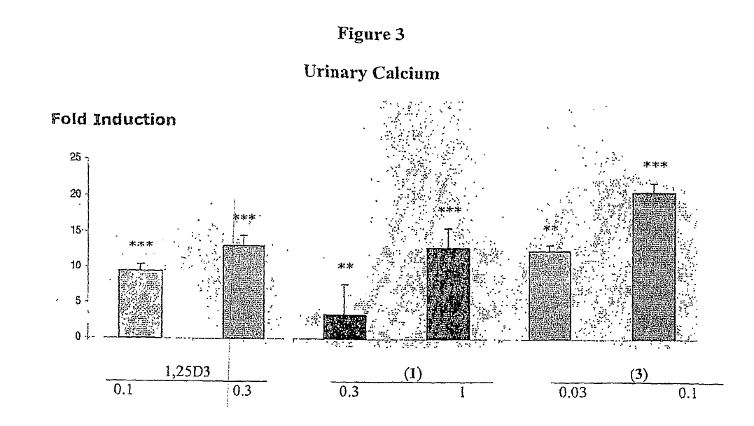

FIG. 3 shows urinary calcium levels in 3 month old OVX rats.

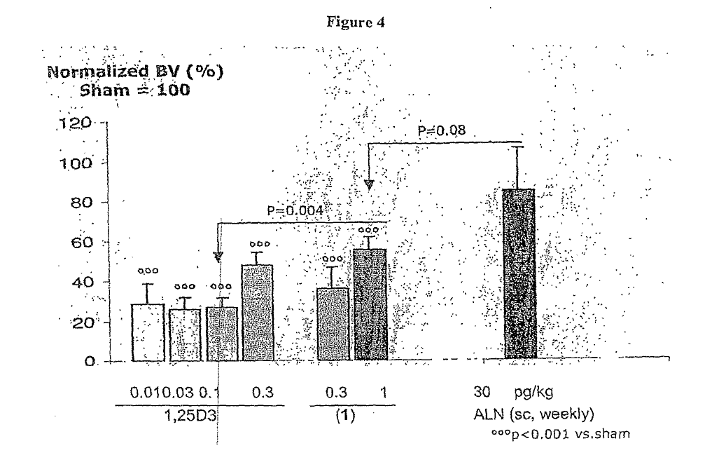

FIG. 4 shows bone volume in 3 month old OVX rats using (1). The efficacy of (1) vs. calcitrol on travecular bone volume was higher at 0.3 μg/kg.

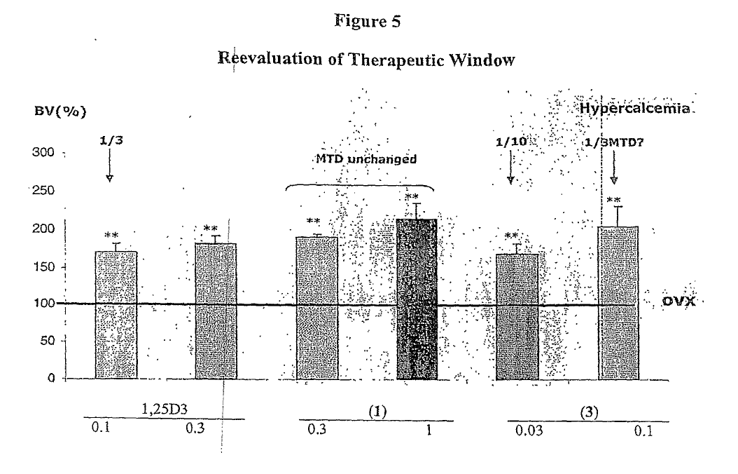

FIG. 5 shows a reevaluation of 3 month old OVX rats for tibia proximal metaphysic bone volume (μCT).

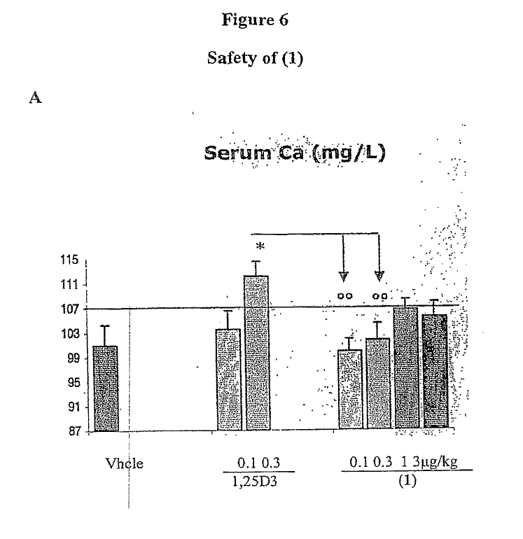

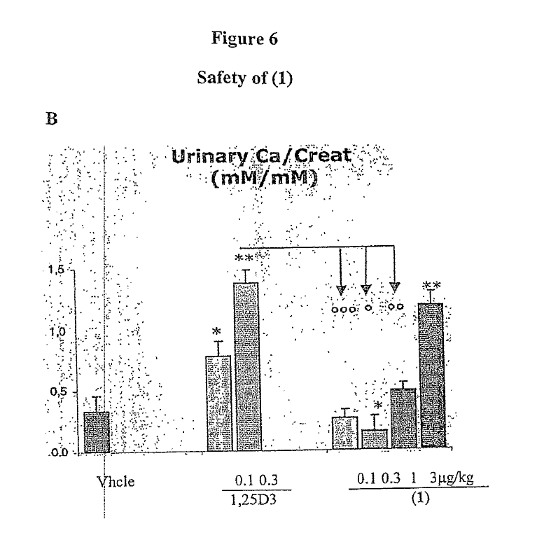

FIG. 6A shows serum Ca levels in 3 month old rats. FIG. 6B shows urinary Ca levels in 3 month old rats. Three month old female rats were orally dosed for three weeks, five days per week, with eight rats per group.

Compounds (1) and (3) demonstrated greater efficacy than calcitriol. Tibia (μCT) was found to be 90% increased over OVX controls with 0.3 μg/kg in rats treated with (1). Tibia (μCT) was found to be 114% increased over OVX controls with 1 μg/kg in rats treated with (1). The vertebrae (L5) was found to have an increase of 8% over OVX control rats when treated with 0.3 μg/kg of (1). The vertebrae (L5) was found to have an increase of 12% over OVX control rats when treated with 1 μg/kg of (1).

Regarding safety, compound (1) provided rats with S Ca>10.7 mg/L in two of sixteen rats at 1 μg/kg.

### Example 10

### Osteoporosis—Long Term Curative Treatment

### Animals

Six month-old Sprague Dawley female rats were purchased from CERJ or Charles River, France. Rats underwent bilateral ovariectomy (OVX) or sham-operation (Sham) under anesthesia with intraperitoneal ketamine hydrochloride (50 mg/kg BW). The success of ovariectomy was evidenced at necropsy by weighing the uterus and visualizing the absence of ovarian tissue. Throughout the whole experiment, rats were housed at 22±2° C. with a 12:12 h light-dark cycle. The animals were pair-fed a standard diet (Safe, 0.6% P, 0.8% Ca) and received Eau de Volvic ad libitum. Experimental procedures were approved by the Animal Ethics Committee of Prostrakan and by DDSV of Seine St Denis, France. The treatment started 8 weeks post-ovariectomy. Compounds were firstly dissolved in ethanol (1 mg/ml). The other dilutions were done in Miglyol 812N. Compounds or vehicle (sham, OVX control rats) were given by daily oral gavage (5 ml/kg), 5/7 days for 8 weeks. Just before operation a group of intact rats were sacrificed for baseline parameters. Before the beginning of treatment, groups of sham and OVX rats were sacrificed to serve as basal controls. Ten and three days before sacrifice, rats were given subcutaneous injections of calcein (10 ml/kg, Sigma) to determine dynamic changes in bone tissue. The day before sacrifice, the rats were fasted and housed in metabolic cages to collect overnight urines.

Evaluation of Bone Parameters: pQCT, μCT, DEXA Analysis

At necropsy, right long bones were removed and fixed in 70% ethanol for further μCT (tibia and femur) and histomorphometry (tibia) analysis. The whole left legs were collected for pQCT analysis. The fourth and fifth lumbar vertebrae were dissected for DXA analysis. Excised tibias were scanned by a ACT machine (Scanco Medical) with software version 3.1 for a 2D-evaluation. The scans started at a distance of 1 mm of the reference line in the proximal tibia metaphysis. Five 0.5 mm-spaced slices were analyzed. The slice thickness was 20-30 μm. Bone volume (BV/TV) and trabeculae number (TbN) and thickness (TbTh) were assessed. Excised tibias were scanned by a pQCT machine (Stratec XCT Research SA+) with software version 5.4 to assess trabecular and cortical Bone Mineral Density (BMD) of proximal tibia metaphysis. For analysis of trabecular bone, the distance between the reference line and the first measurement line was 3 mm. Three other lines separated by 1 mm were analyzed. The cortical BMD was analyzed at 20 mm from the reference line. The voxel size was 0.10 mm. The peel mode used was 20/50. The BMD of excised fourth (L4) and fifth (L5) lumbar vertebrae were scanned by a DXA machine (Hologic QDR 4500) with software optimized for small animal studies. The regional high-resolution software selected a thin X-ray aperture. The large region was 68-71 and the narrow one was 35-21 to assess the body of the vertebrae of L4 and L5.

Histomorphometry Analysis

Left tibia from each rat was removed and dissected free of adjacent tissues. The bones were fixed in 70% ethanol, dehydrated in graded concentrations of ethanol, defatted in xylene, then embedded without decalcification in methyl methacrylate. Five μm-thick sections were made and stained with toluidine blue and cyanin solochrome and used for structural and cellular parameters evaluation. Ten μm-thick sections remained unstained for fluorescence microscopy observations. Structural and dynamic parameters were measured in the secondary spongiosa of the proximal tibial metaphysis situated about 1 mm distal from the growth plate-epiphyseal junction. Structural parameters, i.e. trabecular bone volume BV/TV (%), trabecular thickness (μm), connections and number, as well as cellular parameters (osteoblast, osteoid surfaces and osteoclast number), and dynamic parameters (mineralizing surface, mineral apposition rate, bone formation rate) were evaluated.

### Serum and Urinary Biochemistry

Osteocalcin (IRMA kit, Immutopics), DPyr (Metra DPD EIA kit), CTx (Ratlaps ELISA, Nordic Bioscience Diagnostics) Ca, P, creatinin (Cobas Mira analyser) and rat PTH (Immutopics) were assessed in serum or urine material according to the manufacturer's instructions.

### Results

Data were expressed as mean±sem. Statistics were calculated using StatView version 5.0 for Windows (SAS Institute Inc.). The ANOVA test was used for all groups. Significant difference between groups was determined by Student's t-test. p<0.05 or lower was considered a significant difference.

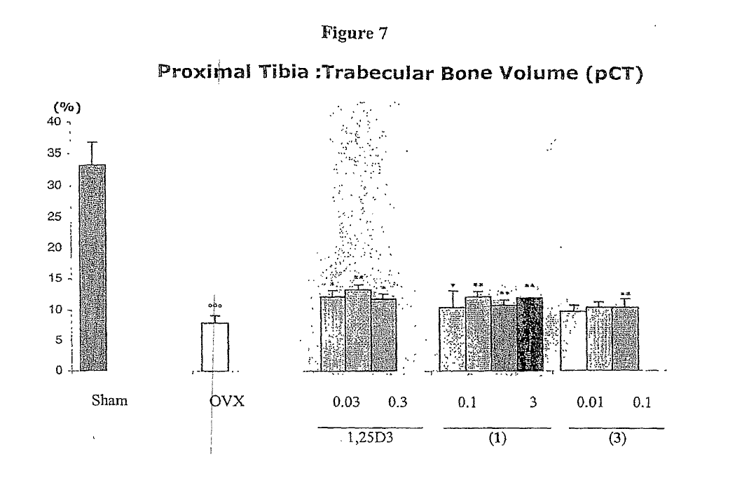

FIG. 7 shows travecular bone volume (μCT) measurements in 6 month old OVX rats.

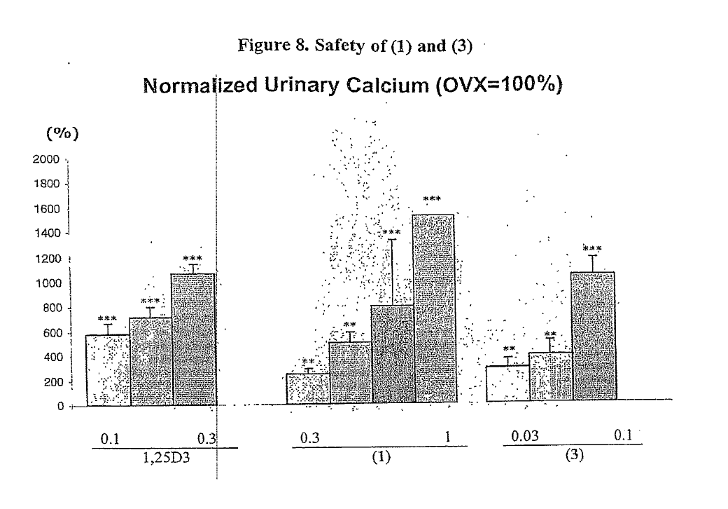

FIG. 8 shows urinary calcium levels in 6 month old OVX rats.

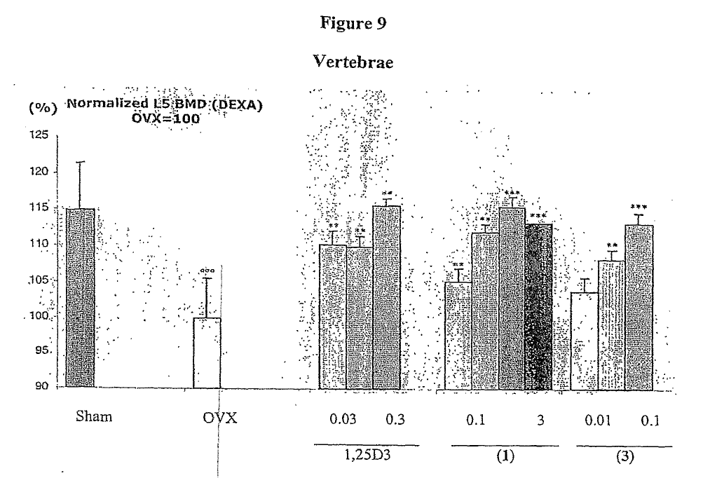

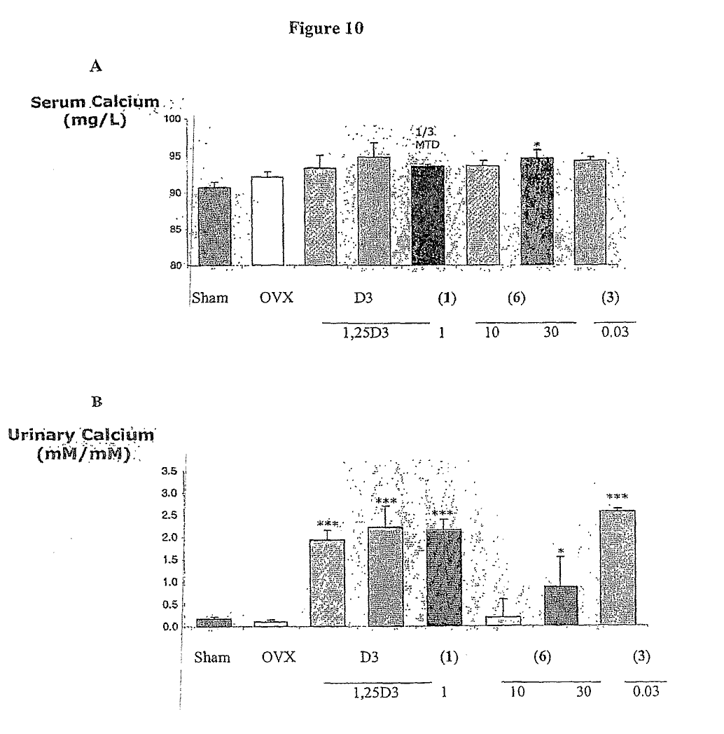

FIG. 9 shows BMD (DEXA) measurements in 6 month old OVX rats.

FIG. 10A shows serum calcium levels. FIG. 10B shows urinary calcium levels.

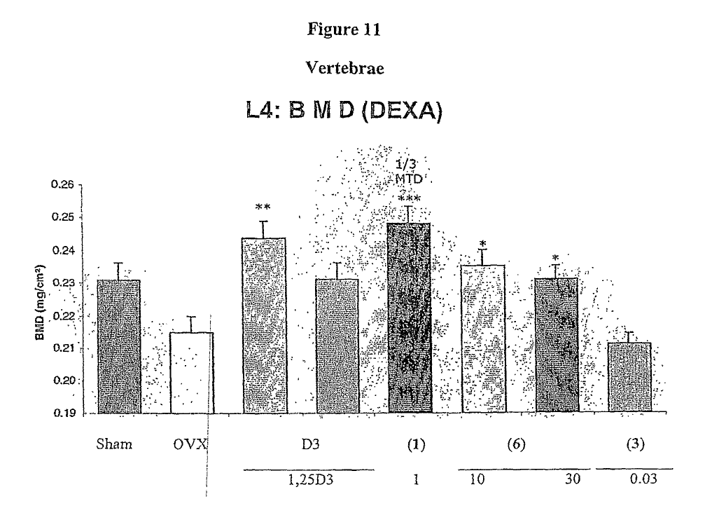

FIG. 11 shows BMD (DEXA) measurements in 6 month old OVX rats.

### Example 11

### Secondary Hyperparathyroidism

### Animals

Three month-old Sprague Dawley male rats (220-250 g BW) were purchased from CERJ, France, Rats were ⅚ nephrectomized (right kidney and ⅔ left kidney removed by surgery) or sham-operated (controls) by the supplier according to Prostrakan'procedures. The success of nephrectomy was evidenced at necropsy by visualizing the kidney tissue and measuring serum creatinin and urea. There were 10-12 rats in each group. The rats were housed at 22±2° C. with a 12 h:12 h light dark cycle. At the beginning of the study, the animals were fed a standard diet (Safe, 0.6% P, 0.8% Ca) and received Eau de Volvic ad libitum. Twenty days after operation, phosphate (Na₂HPO₄) was added to Eau de Volvic (6 g/l). Experimental procedures were approved by the Animal Ethics Committee of Prostrakan and by DDSV of Seine St Denis, France. The treatment started 82 days post-nephrectomy. Compounds were firstly dissolved in ethanol (1 mg/ml). The other dilutions were done in Miglyol 812N. Compounds or vehicle (control rats) were given by daily oral gavage (5 ml/kg), 5/7 days for 49 days. Just before operation a group of intact rats were sacrificed for baseline parameters. Before the beginning of treatment, groups of control and nephrectomized rats were sacrificed to served as basal controls. Ten and three days before sacrifice, rats were given subcutaneous injections of calcein (10 ml/kg, Sigma) to determine dynamic changes in bone tissue. The day before sacrifice, the rats were fasted and housed in metabolic cages to collect overnight urines.

Evaluation of Bone Parameters: pOCT, μCT, DEXA Analysis

At necropsy, right long bones were removed and fixed in 70% ethanol for further μCT (tibia and femur) and histomorphometry (tibia) analysis. The whole left legs were collected for pQCT analysis. The fourth and fifth lumbar vertebrae were dissected for DXA analysis. Excised tibias were scanned by a μCT machine (Scanco Medical) with software version 3.1 for a 2D-evaluation. The scans started at a distance of 1 mm of the reference line in the proximal tibia metaphysis. Five 0.5 mm-spaced slices were analyzed. The slide thickness was 20-30 μm. Bone volume (BV/TV) and trabecular number (TbN) and thickness (TbTh) were assessed. Excised tibias were scanned by a pQCT machine (Stratec XCT Research SA+) with software version 5.4 to assess trabecular and cortical Bone Mineral Density (BMD) of proximal tibia metaphysis. For analysis of trabecular bone, the distance between the reference line and the first measurement line was 3 mm. Three other lines separated by 1 mm were analyzed. The cortical BMD was analyzed at 20 mm from the reference line. The voxel size was 0.10 mm. The peel mode used was 20/50. The BMD of excised fourth (L4) and fifth (L5) lumbar vertebrae were scanned by a DXA machine (Hologic QDR 4500) with software optimized for small animal studies. The regional high-resolution software selected a thin X-ray aperture. The large region was 68-71 and the narrow one was 35-21 to assess the body of the vertebrae of L4 and L5.

### Histomorphometry Analysis

Left tibia from each rat was removed and dissected free of adjacent tissues. The bones were fixed in 70% ethanol, dehydrated in graded concentrations of ethanol, defatted in xylene, then embedded without decalcification in methyl methacrylate. Five μm-thick sections were made and stained with toluidine blue and cyanin solochrome and used for structural and cellular parameters evaluation. Ten μm-thick sections remained unstained for fluorescence microscopy observations. Structural and dynamic parameters were measured in the secondary spongiosa of the proximal tibial metaphysis situated about 1 mm distal from the growth plate-epiphyseal junction. Structural parameters, i.e. trabecular bone volume BV/TV (%), trabecular thickness (μm), connections and number, as well as cellular parameters (osteoblast, osteoid surfaces and osteoclast number), and dynamic parameters (mineralizing surface, mineral apposition rate, bone formation rate) were evaluated.

### Serum and Urinary Biochemistry

Osteocalcin (IRMA kit, Immutopics), DPyr (Metra DPD EIA kit), CTx (Ratlaps ELISA, Nordic Bioscience Diagnostics) Ca, P, creatinin (Cobas Mira analyser) and rat PTH (Immutopics) were assessed in serum or urine material according to the manufacturer's instructions.

### Aortic Calcifications

To assess the calcification, aortas (6 cm-segment starting at arch), hearts and remaining kidney tissues were removed and fixed for histology analysis. Aortas segments were fixed in 3.7% formaldehyde and embedded in paraffin. Five μm cross sections were made and stained with a Von Kossa method for the calcification evaluation. The following grading of the calcification was used: moderate when less 50% of the aortic perimeter was calcified; severe when 100% of the aortic perimeter was calcified.

### Results

Data were expressed as mean±sem. Statistics were calculated using StatView version 5.0 for Windows (SAS Institute Inc.). The ANOVA test was used for all groups. Significant difference between groups was determined by Student's t-test. p<0.05 or lower was considered a significant difference.

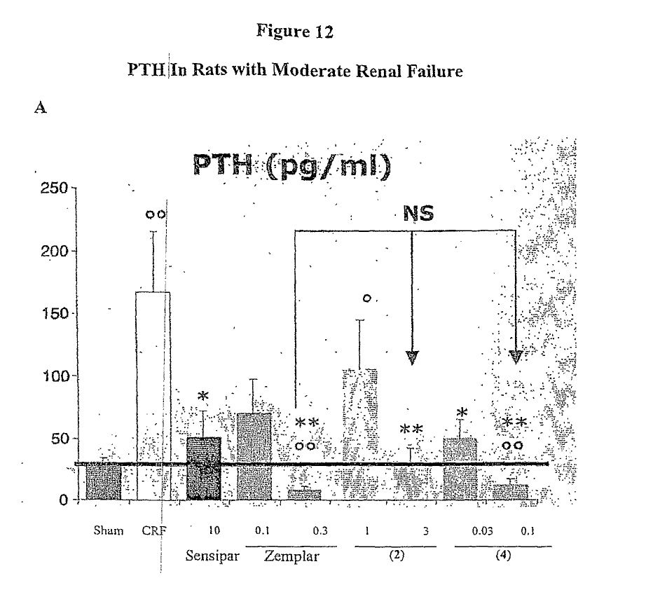

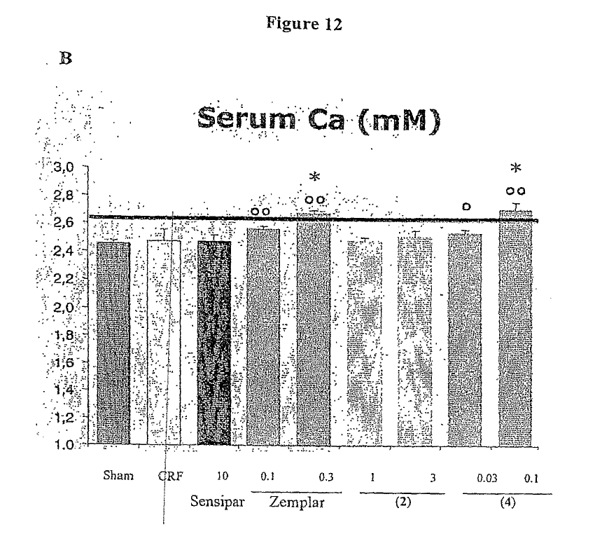

FIG. 12A shows parathyroid hormone (PTH) levels in rats with moderate renal failure. FIG. 12B shows serum Ca levels in rats with moderate renal failure.

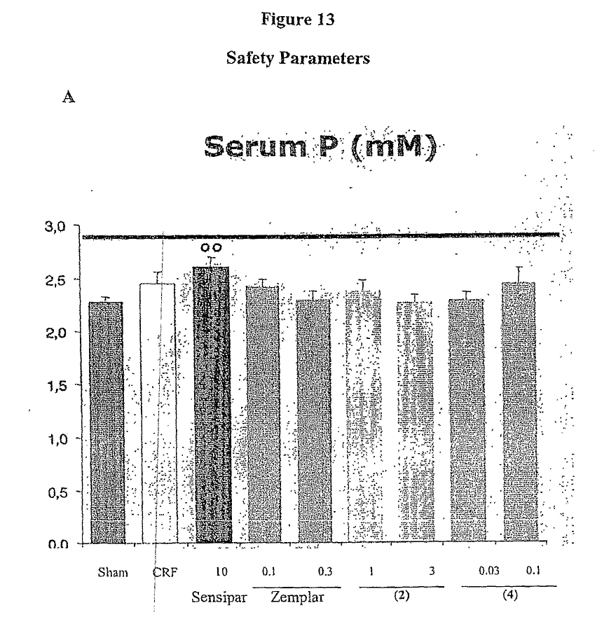

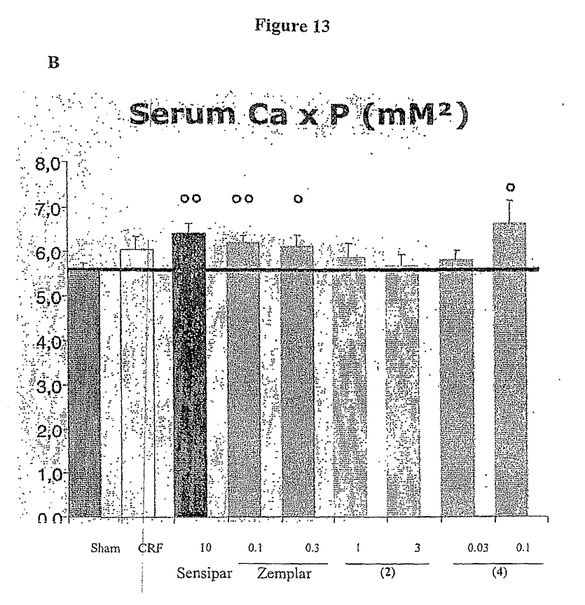

FIGS. 13A and 13B show a model of safety parameters, measuring serum and serum Ca levels.

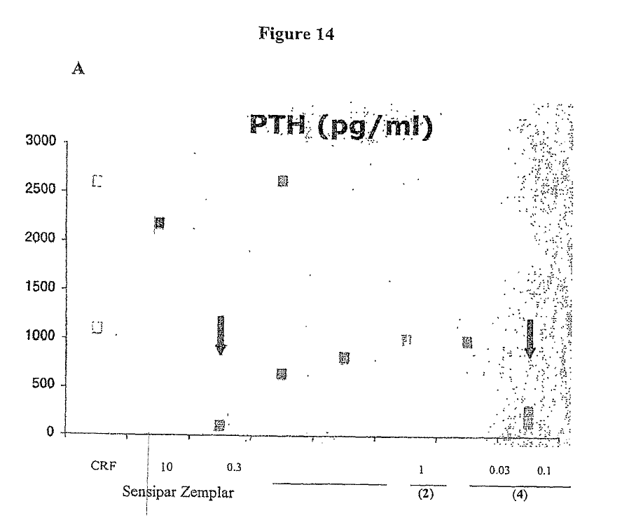

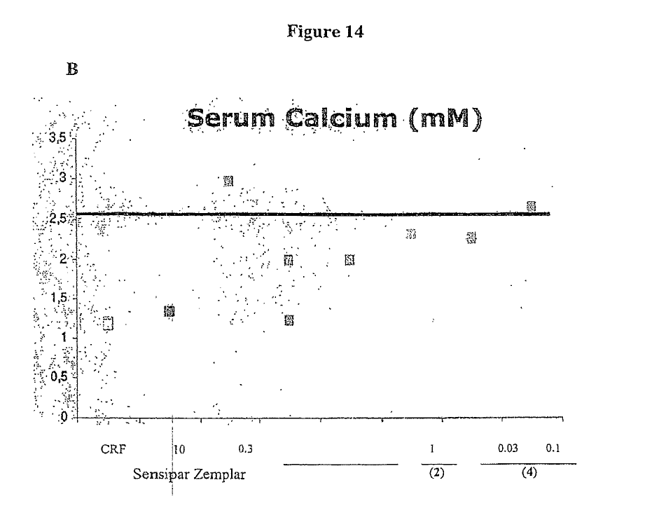

FIG. 14A shows a decrease in PTH levels in rats with severe chronic renal failure. FIG. 14B shows the serum calcium levels in rats with severe chronic renal failure.

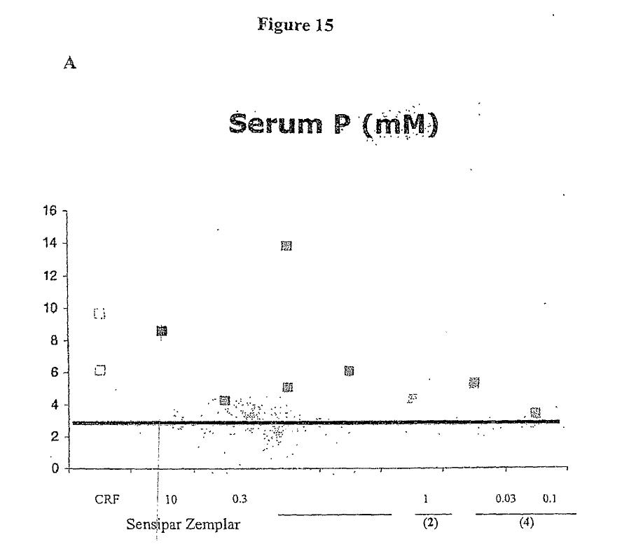

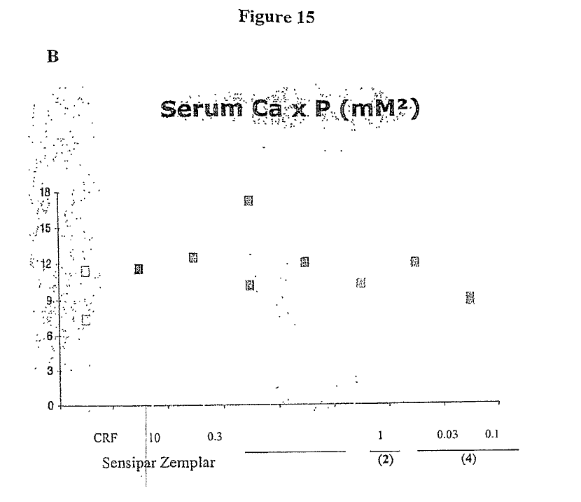

FIGS. 15A and 15B show measurements of serum and serum Ca to determine safety profiles in rats with severe renal failure.

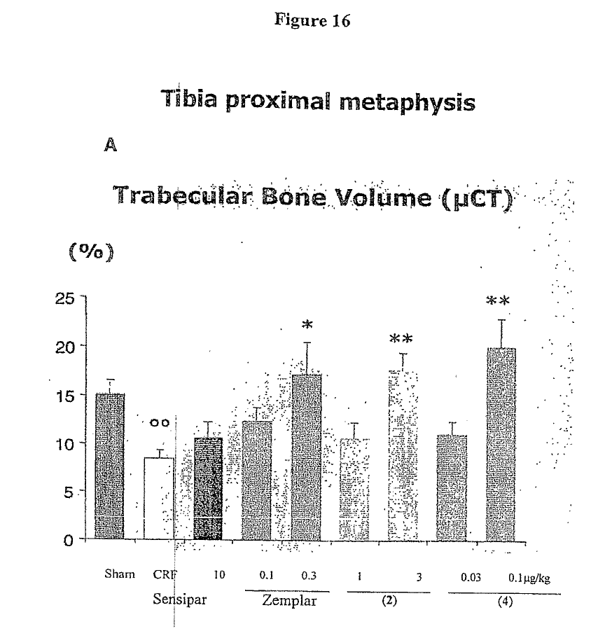

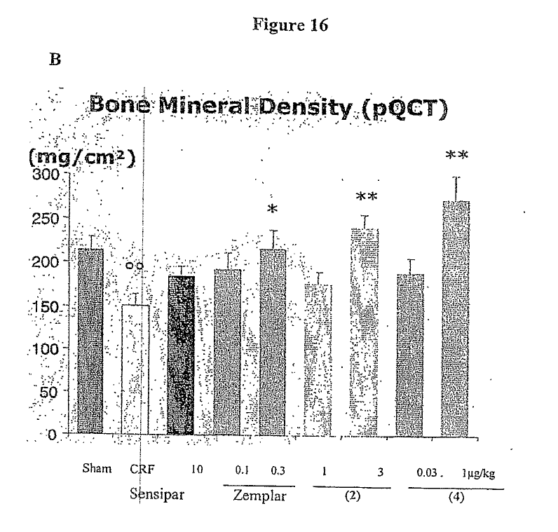

FIG. 16A, shows the trabecular bone volume measurements in uremic rats.

FIG. 16B shows bone mineral density (pQCT) measurements in uremic rats.

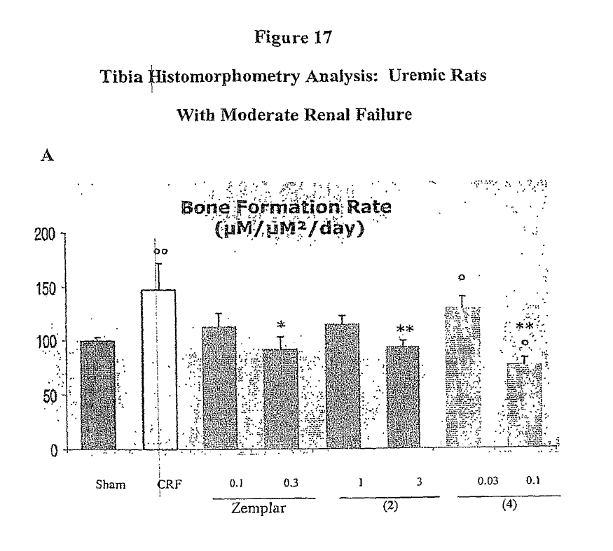

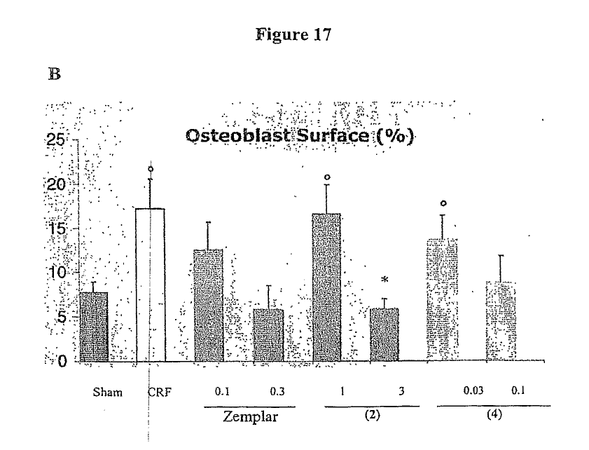

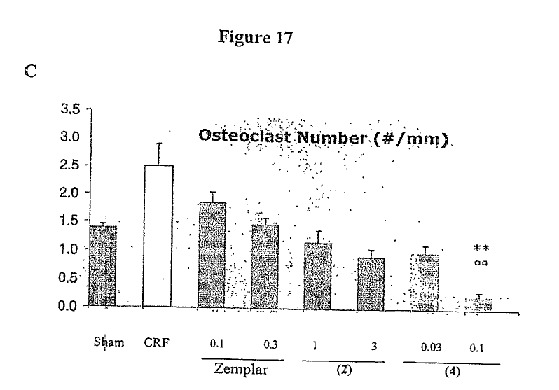

FIGS. 17A, 17B, and 17C show tibia histomorphometry analysis, measuring bone formation rate, osteoblast surface, and osteoclast number in uremic rats with moderate renal failure.

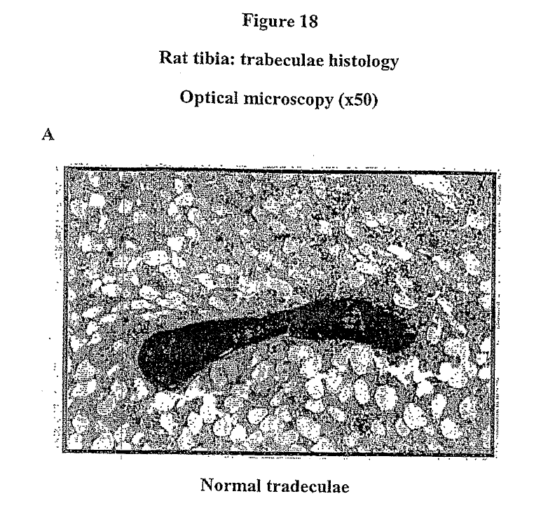

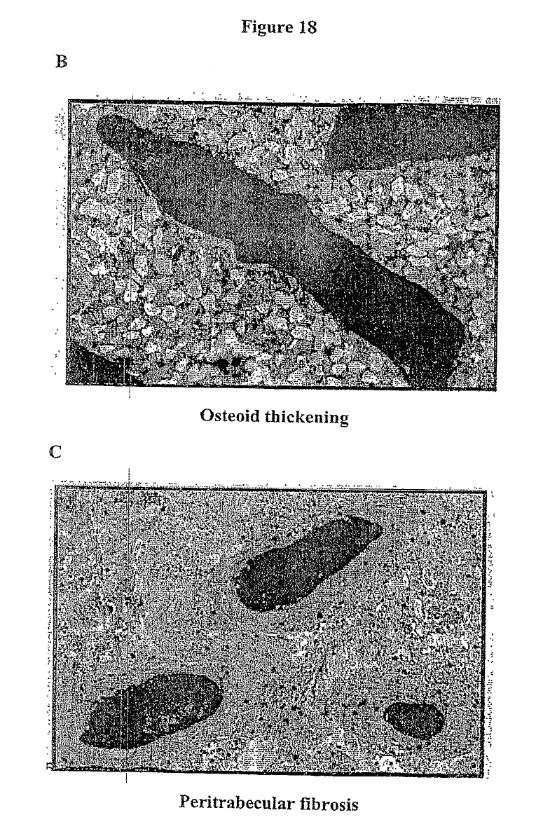

FIG. 18A is a picture of a rat tibia using optical microscopy (×50) of a normal trabeculae. FIG. 18B is a picture of a rat tibia using optical microscopy (×50) of osteoid thickening. FIG. 18C is a picture of a rat tibia using optical microscopy (×50) of peritrabecular fibrosis. Compound (2) provided normal levels of peritrabecular fibrosis at a dose of 3 μg/kg. At 0.1 μg/kg, only one of ten rats demonstrated peritravecular fibrosis. Compound (4) provided normal levels of pentrabecular fibrosis at a dose of 0.1 μg/kg. At 0.03 μg/kg, only one of nine rats demonstrated peritrabecular fibrosis. Compound (2) provided normal levels of osteoid thickening at a dose of 3 μg/kg and 1 μg/kg. Compound (4) provided normal levels of osteoid thickening at a dose of 0.03 μg. At 0.1 μg/kg, only one of nine rats demonstrated osteoid thickening

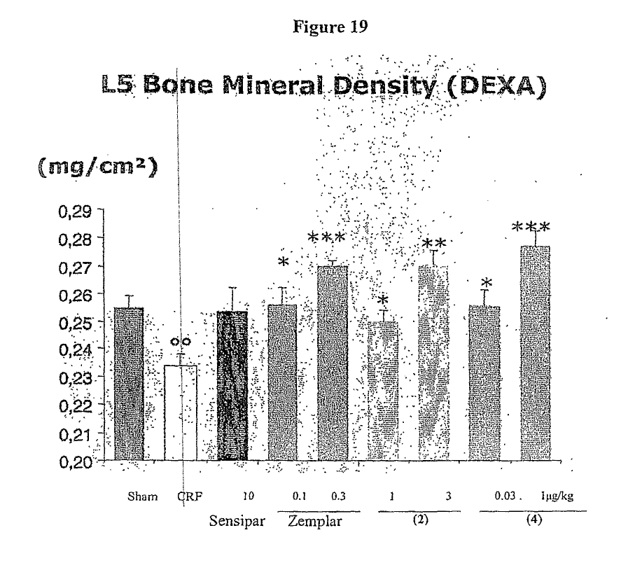

FIG. 19 shows bone mineral density (DEXA) in uremic rats.

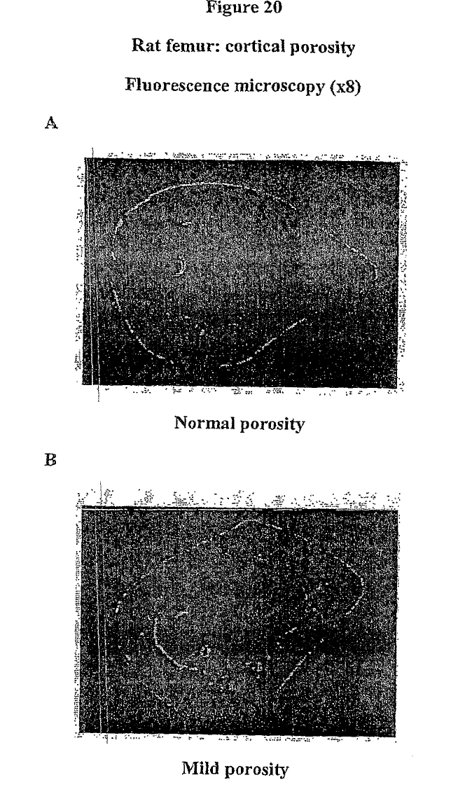

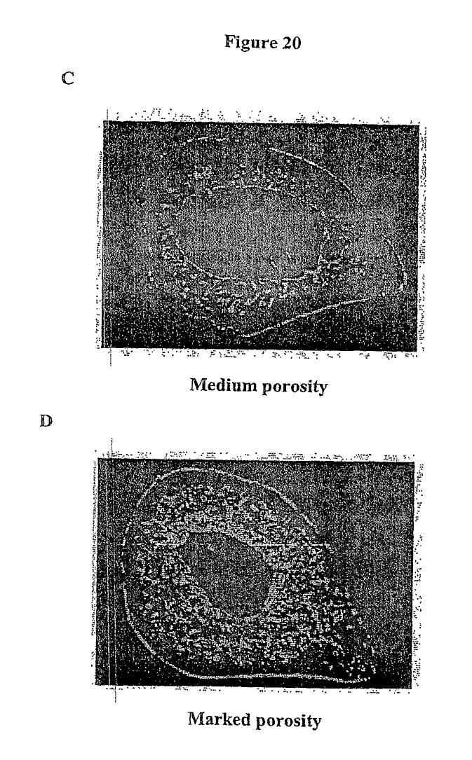

FIG. 20 is a picture of a rat femur cortical porosity using fluorescence microscopy (×1) showing normal porosity, mild porosity, medium porosity, and marked porosity. Compound (2) provided mild porous levels of femur cortical porosity in four of ten rats at 1 μg/kg. At 3 μg/kg, nine rats demonstrated normal levels of femur cortical porosity. Compound (4) provided mild porous levels of femur cortical porosity in four of nine rats at 0.03 μg/kg. Compound (4) provided mild porous levels of femur cortical porosity in one of nine rats at 0.1 μg/kg.

In rats with moderate renal failure, bone loss in CRF rats increased via bone turn over. Compounds (2) and (4) provided bone protection on the tibia and vertebrae; and demonstrated good efficacy.

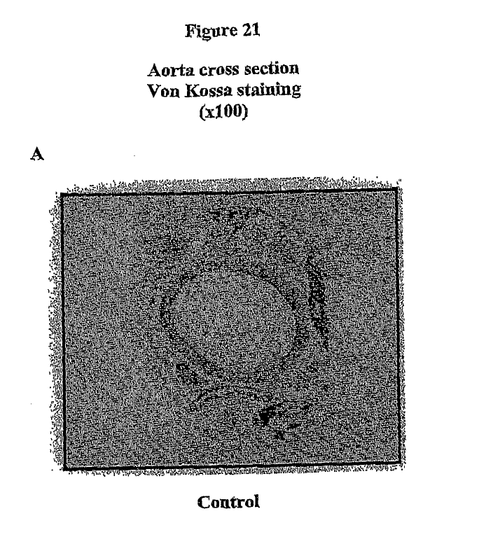

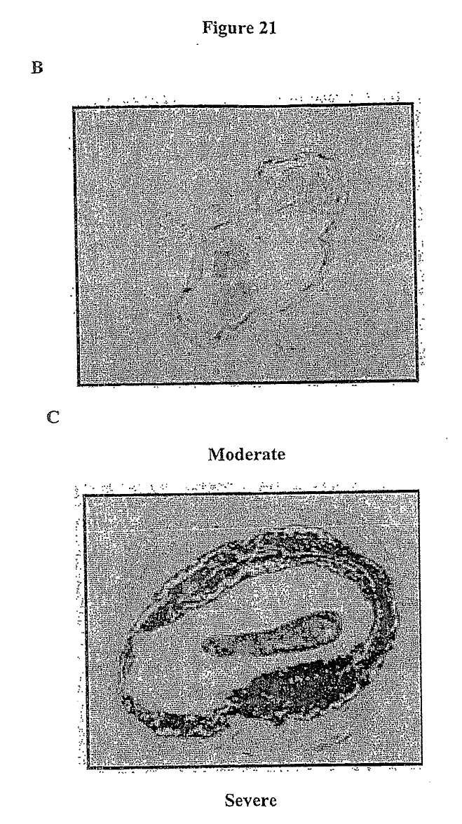

FIG. 21 is a picture of a cross section of a rat aorta using Von Kossa staining (×100), showing a control, moderate aorta calcification, and severe aortic calcification. Compound (4) was administered to seven rats at 0.03 μg/kg which provided uremia of 20.52 mM and S. Creatinin value of 283.23 μM. No calcification was found in CRF control rats, but those rats presenting calcifications had severe renal failure.

Compounds (2) and (4) demonstrated stronger inhibitition of renin in vitro over calcitriol. Both compounds demonstrated efficacy in PTH suppression and bone porosity. Both compounds also demonstrated equal or more beneficial results in terms of safety profiles, when compared to Zemplar. Compounds (2) and (4) demonstrated greater or equal positive results compared to Zemplar in the renin inhibition in vitro experiments, PTH suppression, CaxP, bone porosity, and aortic calcification.

### INCORPORATION BY REFERENCE

The contents of all references (including literature references, issued patents, published patent applications, and co-pending patent applications) cited throughout this application are hereby expressly incorporated herein in their entireties by reference.

### EQUIVALENTS

Those skilled in the art will recognize, or be able to ascertain using no more than routine experimentation, many equivalents of the specific embodiments of the invention described herein. Such equivalents are intended to be encompassed by the following claims.

1. A method for treating osteoporosis in a subject comprising administering to a subject in need thereof a therapeurically effective amount of a vitamin D₃ compound selected from the group consisting of (20S)-1,25-Dihydroxy-20-(5,5,5-trifluoro-4-hydroxy-4-trifluoromethyl-pent-2-ynyl)-cholecalciferol (1); (20S)-1,25-Dihydroxy-20-[(2E)-5,5,5-trifluoro-4-hydroxy-4-trifluoromethyl-pent-2-enyl]-cholecalciferol (3); and (20S)-1α-Fluoro-25-hydroxy-20-(5,5,5-trifluoro-4-hydroxy-4-trifluoromethyl-pent-2-ynyl)-cholecalciferol (6), thereby treating said subject for osteoporosis.

2. The method of claim 1, wherein the vitamin D₃ compound is (20S)-1,25-Dihydroxy-20-(5,5,5-trifluoro-4-hydroxy-4-trifluoromethyl-pent-2-ynyl)-cholecalciferol (1).

3. The method of claim 1, wherein the vitamin D₃ compound is (20S)-1,25-Dihydroxy-20-[(2E)-5,5,5-trifluoro-4-hydroxy-4-trifluoromethyl-pent-2-enyl]-cholecalciferol (3).

4. The method of claim 1, wherein the vitamin D₃ compound is (20S)-1α-Fluoro-25-hydroxy-20-(5,5,5-trifluoro-4-hydroxy-4-trifluoromethyl-pent-2-ynyl)-cholecalciferol (6).

5. The method of claim 1, which further comprises identifying said subject as being in need of treatment for osteoporosis.

6. A method for treating a subject for secondary hyperparathyroidism comprising administering to a subject in need thereof a therapeurically effective amount of a vitamin D₃ compound selected from the group consisting of (20S)-1,25-Dihydroxy-20-((2Z)-5,5,5-trifluoro-4-hydroxy-4-trifluoromethyl-pent-2-enyl)cholecalciferol (2); (20S)-1,25-Dihydroxy-20-[(2E)-5,5,5-trifluoro-4-hydroxy-4-trifluoromethyl-pent-2-enyl]-cholecalciferol (3); (20R)-1,25-Dihydroxy-20-(5,5,5-trifluoro-4-hydroxy-4-trifluoromethyl-pent-2-ynyl)-cholecalciferol (4); and (20R)-1,25-Dihydroxy-20-[(2E)-5,5,5-trifluoro-4-hydroxy-4-trifluoromethyl-pent-2-enyl]-cholecalciferol (5), thereby treating said subject for secondary hyperparathyroidism.

7. The method of claim 6, wherein the vitamin D₃ compound is (20S)-1,25-Dihydroxy-20-((2Z)-5,5,5-trifluoro-4-hydroxy-4-trifluoromethyl-pent-2-enyl)cholecalciferol (2).

8. The method of claim 6, wherein the vitamin D₃ compound is (20S)-1,25-Dihydroxy-20-[(2E)-5,5,5-trifluoro-4-hydroxy-4-trifluoromethyl-pent-2-enyl]-cholecalciferol (3).

9. The method of claim 6, wherein the vitamin D₃ compound is (20R)-1,25-Dihydroxy-20-(5,5,5-trifluoro-4-hydroxy-4-trifluoromethyl-pent-2-ynyl)-cholecalciferol (4).

10. The method of claim 6, wherein the vitamin D₃ compound is (20R)-1,25-Dihydroxy-20-[(2E)-5,5,5-trifluoro-4-hydroxy-4-trifluoromethyl-pent-2-enyl]-cholecalciferol (5).

11. The method of claim 6, which further comprises identifying said subject as being in need of treatment for secondary hyperparathyroidism.

12. The method of claim 1, further comprising obtaining the vitamin D₃ compound.

13. The method of claim 1, wherein the subject is a mammal.

14. The method of claim 13, wherein the subject is human.

15. The method of claim 1, wherein said vitamin D₃ compound is administered to the subject using a pharmaceutically-acceptable formulation.

16. The method of claim 1, wherein said vitamin D₃ compound is administered in combination with a pharmaceutically acceptable diluent or carrier.

17. The method of claim 15, wherein said pharmaceutically-acceptable formulation provides sustained delivery of said vitamin D₃ compound to a subject for at least four weeks after the pharmaceutically-acceptable formulation is administered to the subject.

18. The method of claim 1, wherein said compound is administered orally, intravenously, topically, or parenterally.

19. The method of claim 1, wherein said compound is administered at a concentration of 0.001 μg-100 μg/kg of body weight.

20. A pharmaceutical composition for use in the treatment of osteroporosis, comprising a therapeutically effective amount of a vitamin D₃ compound selected from the group consisting of (20S)-1,25-Dihydroxy-20-(5,5,5-trifluoro-4-hydroxy-4-trifluoromethyl-pent-2-ynyl)-cholecalciferol (1); (20S)-1,25-Dihydroxy-20-[(2E)-5,5,5-trifluoro-4-hydroxy-4-trifluoromethyl-pent-2-enyl]-cholecalciferol (3); and (20S)-1α-Fluoro-25-hydroxy-20-(5,5,5-trifluoro-4-hydroxy-4-trifluoromethyl-pent-2-ynyl)-cholecalciferol (6), and a pharmaceutically acceptable diluent or carrier.

21-23. (canceled)

24. A pharmaceutical composition for use in the treatment of secondary hyperparathyroidism comprising a therapeutically effective amount of a vitamin D₃ compound according to claim 6, and a pharmaceutically acceptable diluent or carrier.

25-28. (canceled)

29. A packaged formulation for use in the treatment of osteoporosis, comprising a pharmaceutical composition comprising a vitamin D₃ compound according to claim 1; and instructions for use in the treatment of osteoporosis.

30-32. (canceled)

33. A packaged formulation for use in the treatment of secondary hyperparathyroidism, comprising a pharmaceutical composition comprising a vitamin D₃ compound according to claim 6, and instructions for use in the treatment of secondary hyperparathyroidism.

34-37. (canceled)
